(19) United States
(12) Patent Application Publication
(10) Pub. No.: US 2010/0297079 A1
(43) Pub. Date: Nov. 25, 2010

(54) COMPOUNDS, COMPOSITIONS AND METHODS FOR TREATING VIRAL INFECTION

(75) Inventors: Merrick Almond, Apex, NC (US); Ernest R. Lanier, Chapel Hill, NC (US); David L. Musso, Raleigh, NC (US); Roy Ware, Raleigh, NC (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Chimerix, Inc., Durham, NC (US)

(21) Appl. No.: 12/784,000

(22) Filed: May 20, 2010

Related U.S. Application Data
(60) Provisional application No. 61/179,958, filed on May 20, 2009.

(51) Int. Cl. A61K 38/21; C07H 19/14; A61K 31/7064; A61P 31/12; A61K 31/7068; A61K 38/07; A61K 38/05

(52) U.S. Cl. 424/85.7; 536/27.2; 514/49; 536/26.7; 536/26.14; 514/3.7

(57) ABSTRACT

The present invention describes compounds of formulae I and II and methods for treating viral infection, such as Flaviviridae virus infection, including Hepatitis C infection (HCV).

### RELATED APPLICATIONS

This application claims priority to U.S. Provisional Application Ser. No. 61/179,958, filed May 20, 2009, the entire contents of which are incorporated herein by reference.

### FIELD OF THE INVENTION

The present invention generally relates to nucleoside compounds, derivatives and analogues thereof and methods for treating viral infection, for example, compounds of the invention may be used to treat Flaviviridae virus infection, such as Hepatitis C infection (HCV).

### BACKGROUND OF THE INVENTION

Hepatitis C Virus (HCV) is one of the most prevalent causes of chronic liver disease such as cirrhosis and hepatocellular carcinoma. More than 4 million Americans (1.3% of the U.S. population) and 170 million individuals in the world (3% worldwide) are infected with hepatitis C virus.

HCV is a small enveloped positive-strand RNA flavivirus containing a genome of about 10 kilobases. The genome has a single uninterrupted ORF (open reading frame) that encodes a protein of 3010-3011 amino acids. The structural proteins of HCV include a core protein (C), which is highly immunogenic, as well as two envelope proteins (E1 and E2), which likely form a heterodimer in vivo, and non-structural proteins NS2-NS5. It is known that the NS3 region of the virus is important for post-translational processing of the polyprotein into individual proteins, and the NS5 region encodes an RNA-dependant RNA polymerase.

There is currently no vaccine for HCV and the standard of care therapy (pegylated interferon plus ribavirin) provides a durable response for approximately 40 to 50% of patients with HCV subtype 1 or 4. However, this therapy has significant adverse effects. New approaches for HCV treatment are focused on combining inhibitors of viral enzymes with the hope of using multiple antiviral agents together to improve responses to existing therapy and/or replace interferon based therapy (Soriano et al, Clinical Infectious Disease 48:313-320, 2009). However, these strategies are complicated by the inherent diversity of viral genotypes, leading to rapid emergence of resistant strains, and the need for multiple agents acting on different targets.

In light of the challenging nature of HCV infection, there is an on-going need to develop effective therapeutics for treatment of HCV infection.

### SUMMARY OF THE INVENTION

The present invention provides compounds of Formula I or Formula II

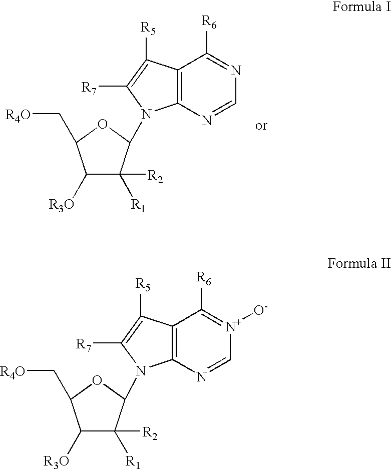

wherein:

R₁ and R₂ are independently selected from the group consisting of halogen, hydrogen, hydroxyl, N₃, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl and —NR′R″, wherein each occurrence of R′ and R″ are independently selected from the group consisting of hydrogen, hydroxyl, unsubstituted or substituted C₁₋₈ alkyl, unsubstituted or substituted C₁₋₈ alkoxyl, and unsubstituted or substituted C₃₋₆ cycloalkyl;

R₁ and R₂ are independently ORₓ or OR_{y}; or

R₁ forms an unsubstituted or substituted 5-7 member ring with R₃ wherein said ring optionally comprises 1-2 additional heteroatoms selected from N, O or S; and

R₃, R₄, Rₓ and R_{y} are independently selected from the group consisting of

(a) hydrogen,

(b) unsubstituted or substituted C₁₋₈ alkyl, —C(═O)—Rₐ, —C(═O)—ORₐ or —C(═O)—NRₐRₐ′ wherein each occurrence of Rₐ and Rₐ′ are independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S;

(c) monophosphate, diphosphate or triphosphate,

(d) a moiety of Formula A, A′ or A″:

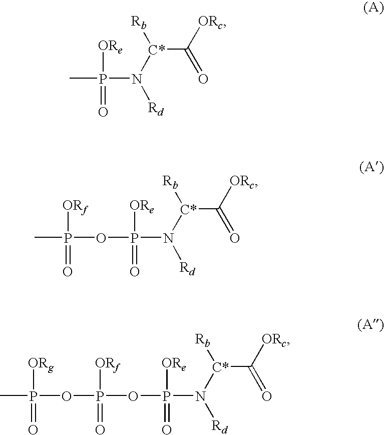

wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,

wherein R_{z} is hydrogen or unsubstituted or substituted C₁₋₈ alkyl;

R_{c}, R_{d}, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,

Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,

R_{b}, R_{d}, C* and N may form an unsubstituted or substituted 4-6 membered heterocycle comprising 1-3 additional heteroatoms selected from N, O or S;

(e) an amino acyl moiety of an amino acid;

(f) a moiety of Formula B, B′ or B″; and

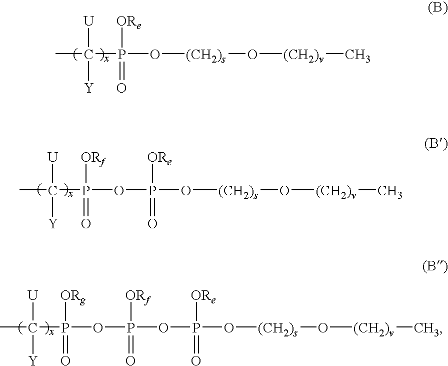

wherein U and Y are independently H or halogen, x is 0, 1 or 2, s is an integer from 2 to 6, v is an integer from 11 to 25, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S and Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S; or

R₃ and R₄ form a 5′,3′-cyclic phosphate as shown in Formula E or E′:

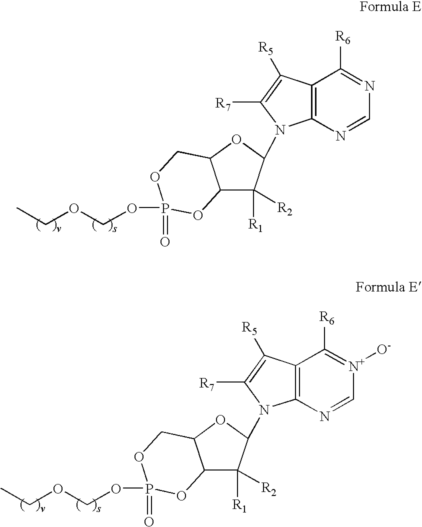

wherein s is an integer from 2 to 6, v is an integer from 11 to 25 and R₁, R₂, R₅, R₆ and R₇ are as described herein; and

R₅, R₆ and R₇ are independently selected from the group consisting of hydrogen, halogen, hydroxyl, CN, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl, unsubstituted or substituted C₁₋₈ thioalkyl, unsubstituted or substituted C₃₋₆ cycloalkyl, unsubstituted or substituted C₃₋₆ cycloalkenyl, unsubstituted or substituted C₆₋₁₄ aryl, unsubstituted or substituted heteroaryl comprising 1-4 heteroatoms selected from N, O and S, and —NRⁱRⁱⁱ wherein at each occurrence Rⁱ and Rⁱⁱ are independently selected from the group consisting of hydrogen, hydroxyl, unsubstituted or substituted C₁₋₈ alkyl, unsubstituted or substituted C₁₋₈ alkenyl, unsubstituted or substituted C₁₋₈ alkynyl, unsubstituted or substituted C₁₋₈ alkoxyl and unsubstituted or substituted C₃₋₆ cycloalkyl, or

R₅, R₆ and R₇ are independently Formula C:

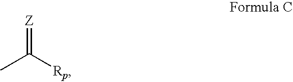

wherein Z is selected from the group consisting of O, S and NRⱼ, wherein Rⱼ is hydrogen, hydroxyl or unsubstituted or substituted C₁₋₈ alkoxyl; Rₚ is hydrogen, unsubstituted or substituted C₁₋₈ alkoxyl or —NRₘRₙ, wherein each occurrence of Rₘ, or Rₙ are independently hydrogen, hydroxyl, unsubstituted or substituted C₁₋₈ alkyl, or unsubstituted or substituted C₁₋₈ alkoxyl;

wherein said cycloalkyl, cycloalkenyl, heterocycle, aryl or heteroaryl may optionally attach via a C₁₋₈ alkyl or C₁₋₈ alkoxyl linker;

or a pharmaceutically acceptable salt, prodrug, tautomer, regioisomer, stereoisomer, diastereomer, enantiomer or racemate thereof.

In one embodiment, compounds of the present invention have the structure of Formula Ix. Formula Iy or Formula Iz:

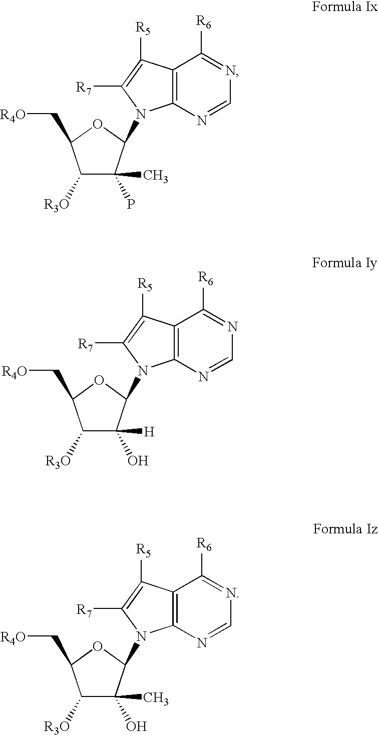

Another aspect of the present invention provides pharmaceutical compositions comprising a therapeutically effective amount of a compound described herein and a pharmaceutically acceptable carrier.

Further, one aspect of the present invention provides methods for treating Hepatitis C virus (HCV) infection in a subject in need of such treatment, the method comprising administering to said subject a therapeutically effective amount of a compound described herein or a combination of compounds described herein. In one embodiment, methods described herein may combine with a therapeutically effective amount of at least one additional therapeutically active agent against HCV.

Objects of the present invention will be appreciated by those of ordinary skill in the art from a reading of the Figures and the detailed description of the preferred embodiments which follow, such description being merely illustrative of the present invention.

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms of “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Generally, the nomenclature used herein and the laboratory procedures in organic chemistry, medicinal chemistry, biology and virology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

All patents, patent applications and publications referred to herein are incorporated by reference in their entirety. In case of a conflict in terminology, the present specification is controlling.

### A. DEFINITIONS

As used herein, “alkyl”, “C₁, C₂, C₃, C₄, C₅, C₆, C₇ or C₈ alkyl” or “C₁-C₈ alkyl” is intended to include C₁, C₂, C₃, C₄, C₅, C₆, C₇ or C₈ straight chain (linear) saturated aliphatic hydrocarbon groups and C₃, C₄, C₅, C₆, C₇ or C₈ branched saturated aliphatic hydrocarbon groups. For example, C₁-C₈ alkyl is intended to include C₁, C₂, C₃, C₄, C₅, C₆, C₇ and C₈ alkyl groups. Alkyl can also include e.g., C₁₋₆ alkyl, C₁₋₅ alkyl, C₁₋₄ alkyl, C₁₋₃ alkyl or C₁₋₂ alkyl. Examples of alkyl include, moieties having from one to eight carbon atoms, such as, but not limited to, methyl, ethyl, n-propyl, i-propyl, n-butyl, s-butyl, t-butyl, n-pentyl, s-pentyl, n-hexyl, n-heptyl, or n-octyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

“Heteroalkyl” groups are alkyl groups, as defined above, that have an oxygen, nitrogen, sulfur or phosphorous atom replacing one or more hydrocarbon backbone carbon atoms.

As used herein, the term “cycloalkyl”, “C₃, C₄, C₅, C₆, C₇ or C₈ cycloalkyl” or “C₃-C₈ cycloalkyl” is intended to include hydrocarbon rings having from three to eight carbon atoms in their ring structure. In one embodiment, a cycloalkyl group has five or six carbons in the ring structure.

The term “substituted alkyl” refers to alkyl moieties having substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)).

Unless the number of carbons is otherwise specified, “lower alkyl” includes an alkyl group, as defined above, having from one to six, or, in another embodiment from one to four, carbon atoms in its backbone structure. “Lower alkenyl” and “lower alkynyl” have chain lengths of, for example, two to six or of two to four carbon atoms.

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), branched alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched alkenyl group has six or fewer carbon atoms in its backbone (e.g., C₂-C₆ for straight chain, C₃-C₆ for branched chain). Likewise, cycloalkenyl groups may have from five to eight carbon atoms in their ring structure, and in one embodiment, cycloalkenyl groups have five or six carbons in the ring structure. The term “C₂-C₈” includes alkenyl groups containing two to eight carbon atoms. The term “C₃-C₈” includes alkenyl groups containing three to eight carbon atoms.

“Heteroalkenyl” includes alkenyl groups, as defined herein, having an oxygen, nitrogen, sulfur or phosphorous atom replacing one or more hydrocarbon backbone carbons.

The term “substituted alkenyl” refers to alkenyl moieties having substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), branched alkynyl groups, and cycloalkyl or cycloalkenyl substituted alkynyl groups. In certain embodiments, a straight chain or branched alkynyl group has six or fewer carbon atoms in its backbone (e.g., C₂-C₆ for straight chain, C₃-C₆ for branched chain). The term “C₂-C₈” includes alkynyl groups containing two to eight carbon atoms. The term “C₃-C₈” includes alkynyl groups containing three to eight carbon atoms.

“Heteroalkynyl” includes alkynyl groups, as defined herein, having an oxygen, nitrogen, sulfur or phosphorous atom replacing one or more hydrocarbon backbone carbons.

The term “substituted alkynyl” refers to alkynyl moieties having substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aryl” includes groups with aromaticity, including “conjugated”, or multicyclic, systems with at least one aromatic ring. Examples include phenyl, benzyl, etc.

“Heteroaryl” groups are aryl groups, as defined above, having from one to four heteroatoms in the ring structure, and may also be referred to as “aryl heterocycles” or “heteroaromatics”. As used herein, the term “heteroaryl” is intended to include a stable 5-, 6-, or 7-membered monocyclic or 7-, 8-, 9-, 10-, 11-, 12-, 13- or 14-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The aryl or heteroaryl aromatic ring can be substituted at one or more ring positions with such substituents as described above, for example, alkyl, alkenyl, akynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to include any stable monocyclic, bicyclic or tricyclic ring having the specified number of carbons, any of which may be saturated, unsaturated, or aromatic. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” includes any ring structure (saturated or partially unsaturated) which contains at least one ring heteroatom (e.g., N, O or S). Examples of heterocycles include, but are not limited to, morpholine, pyrrolidine, tetrahydrothiophene, piperidine, piperazine and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted”, as used herein, means that any one or more hydrogen atoms on the designated atom is replaced with a selection from the indicated groups, provided that the designated atom's normal valency is not exceeded, and that the substitution results in a stable compound. When a substituent is keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

Combinations of substituents and/or variables are permissible, but only if such combinations result in stable compounds.

The term “hydroxy” or “hydroxyl” includes groups with an —OH or the deprotonated form, —O⁻.

As used herein, “halo” or “halogen” refers to fluoro, chloro, bromo and iodo. The term “perhalogenated” generally refers to a moiety wherein all hydrogen atoms are replaced by halogen atoms.

The term “carbonyl” or “carboxy” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

“Acyl” includes moieties that contain the acyl radical (—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl”, “alkylaminoalkyl” and “thioalkoxyalkyl” include alkyl groups, as described above, wherein oxygen, nitrogen or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” includes compounds or moieties which contain an oxygen atom bonded to two carbon atoms or heteroatoms. For example, the term includes “alkoxyalkyl”, which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls and alkthioalkynyls. The term “alkthioalkyls” include moieties with an alkyl, alkenyl or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” includes moieties where a nitrogen atom is covalently bonded to at least one carbon or heteroatom. “Alkylamino” includes groups of compounds wherein nitrogen is bound to at least one alkyl group. Examples of alkylamino groups include benzylamino, methylamino, ethylamino, phenethylamino, etc. “Dialkylamino” includes groups wherein the nitrogen atom is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Alkylarylamino”, “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

As used herein, the term “amino acid” refers to a compound comprising a primary amino (—NH2) group and a carboxylic acid (—COOH) group. The amino acids used in the present invention include naturally occurring and synthetic α, β, γ or δ amino acids, and includes but are not limited to, amino acids found in proteins. Exemplary amino acids include, but are not limited to, glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In some embodiments, the amino acid may be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-argininyl or β-histidinyl. Additionally, as used herein, “amino acids” also include derivatives of amino acids such as esters, and amides, and salts, as well as other derivatives, including derivatives having pharmacoproperties upon metabolism to an active form.

As used herein, the term “natural a amino acid” refers to a naturally occurring a-amino acid comprising a carbon atom bonded to a primary amino (—NH₂) group, a carboxylic acid (—COOH) group, a side chain, and a hydrogen atom. Exemplary natural a amino acids include, but are not limited to, glycine, alanine, valine, leucine, isoleucine, methionine, phenylalanine, tryptophane, proline, serine, threonine, cysteine, tyrosine, asparaginate, glutaminate, aspartate, glutamate, lysine, arginine and histidine.

As used herein, “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects, and further including various races and ethnicities including, but not limited to, white, black, Asian, American Indian and Hispanic.

As used herein, “treatment”, “treat”, and “treating” refer to reversing, alleviating, inhibiting the progress, or delaying the progression of a disorder or disease as described herein.

As used herein, “prevention”, “prevent”, and “preventing” describes reducing or eliminating the onset of the symptoms or complications of the disease, condition or disorder.

As used herein “an effective amount” refers to an amount that causes relief of symptoms of a disorder or disease as noted through clinical testing and evaluation, patient observation, and/or the like. An “effective amount” can further designate a dose that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition of interest. In some embodiments, an “effective amount” can further refer to a therapeutically effective amount.

Furthermore, it will be appreciated by one of ordinary skill in the art that the synthetic methods, as described herein, utilize a variety of protecting groups. As used herein, the term “protecting group” refers to a particular functional moiety, e.g., O, S, or N, that is temporarily blocked so that a reaction can be carried out selectively at another reactive site in a multifunctional compound. Protecting groups may be introduced and removed at appropriate stages during the synthesis of a compound using methods that are known to one of ordinary skill in the art. The protecting groups are applied according to standard methods of organic synthesis as described in the literature (Theodora W. Green and Peter G. M. Wuts (2007) Protecting Groups in Organic Synthesis, 4^{th} edition, John Wiley and Sons, incorporated by reference with respect to protecting groups).

Exemplary protecting groups include, but are not limited to, oxygen, sulfur, nitrogen and carbon protecting groups. For example, oxygen protecting groups include, but are not limited to, methyl ethers, substituted methyl ethers (e.g., MOM (methoxymethyl ether), MTM (methylthiomethyl ether), BOM (benzyloxymethyl ether), PMBM (p-methoxybenzyloxymethyl ether), optionally substituted ethyl ethers, optionally substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), esters (e.g., formate, acetate, benzoate (Bz), trifluoroacetate, dichloroacetate) carbonates, cyclic acetals and ketals. In addition, exemplary nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enaminc derivatives, etc. Certain other exemplary protecting groups are detailed herein, however, it will be appreciated that the present invention is not intended to be limited to these protecting groups; rather, a variety of additional equivalent protecting groups may be utilized according to methods known to one skilled in the art.

### B. COMPOUNDS

According to some aspects of the present invention, novel compounds with a range of biological properties are provided. Compounds described herein have biological activities relevant for the treatment of Flaviviridae infections, in particular hepatitis C(HCV) virus infection.

According to one aspect of the present invention, provided herein are compounds of Formula I:

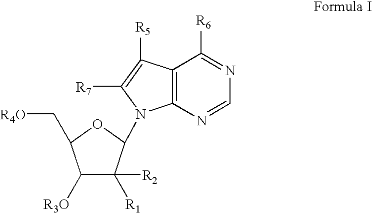

wherein:

R₁ and R₂ are independently selected from the group consisting of halogen, hydrogen, hydroxyl, N₃, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl and —NR′R″, wherein each occurrence of R′ and R″ are independently selected from the group consisting of hydrogen, hydroxyl, unsubstituted or substituted C₁₋₈ alkyl, unsubstituted or substituted C₁₋₈ alkoxyl, and unsubstituted or substituted C₃₋₆ cycloalkyl; or

R₁ and R₂ are independently ORₓ, or OR_{y}; or

R₁ forms an unsubstituted or substituted 5-7 member ring with R₃ wherein said ring optionally comprises 1-2 additional heteroatoms selected from N, O or S; and

R₃, R₄, Rₓ and R_{y} are independently selected from the group consisting of

(a) hydrogen,

(b) unsubstituted or substituted C₁₋₈ alkyl, —C(═O)—Rₐ, —C(═O)—ORₐ or —C(═O)—NRₐRₐ′ wherein each occurrence of Ra and Ra′ are independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S;

(c) monophosphate, diphosphate or triphosphate,

(d) a moiety of Formula A, A′ or A″:

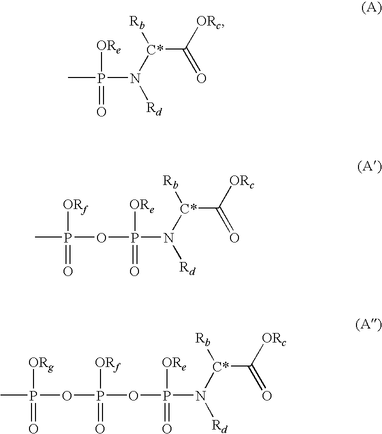

wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,

wherein R_{z} is hydrogen or unsubstituted or substituted C₁₋₈ alkyl;

R_{c}, R_{d}, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,

Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,

R_{b} R_{d}, C* and N may form an unsubstituted or substituted 4-6 membered heterocycle comprising 1-3 additional heteroatoms selected from N, O or S;

(e) an amino acyl moiety of an amino acid;

(f) a moiety of Formula B, B′ or B″;

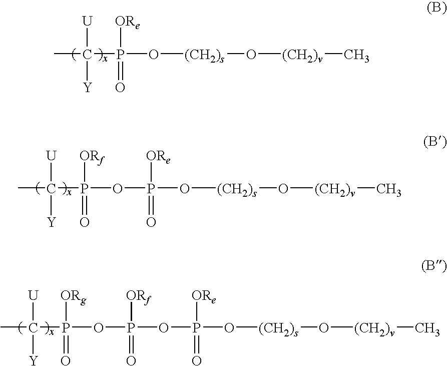

wherein U and Y are independently H or halogen, x is 0, 1 or 2, s is an integer from 2 to 6, v is an integer from 11 to 25, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S, and Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S; or

R₃ and R₄ form a 5′,3′-cyclic phosphate as shown in Formula E:

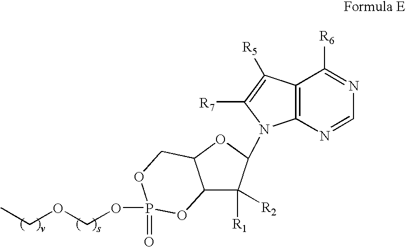

wherein s is an integer from 2 to 6, v is an integer from 11 to 25 and R₁, R₂, R₅, R₆ and R₇ are as described herein; and

R₅, R₆ and R₇ are independently selected from the group consisting of hydrogen, halogen, hydroxyl, CN, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl, unsubstituted or substituted C₁₋₈ thioalkyl, unsubstituted or substituted C₃₋₆ cycloalkyl, unsubstituted or substituted C₃₋₆ cycloalkenyl, unsubstituted or substituted C₆₋₁₄ aryl, unsubstituted or substituted heteroaryl comprising 1-4 heteroatoms selected from N, O and S, and —NRⁱRⁱⁱ, wherein at each occurrence Rⁱ and Rⁱⁱ are independently selected from the group consisting of hydrogen, hydroxyl, unsubstituted or substituted C₁₋₈ alkyl, unsubstituted or substituted C₁₋₈ alkenyl, unsubstituted or substituted C₁₋₈ alkynyl, unsubstituted or substituted C₁₋₈ alkoxyl and unsubstituted or substituted C₃₋₆ cycloalkyl, or

R₅, R₆ and R₇ are independently Formula C:

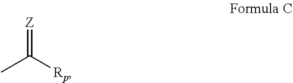

wherein Z is selected from the group consisting of O, S and NRⱼ, wherein Rⱼ is hydrogen, hydroxyl or unsubstituted or substituted C₁₋₈ alkoxyl; Rₚ is hydrogen, unsubstituted or substituted C₁₋₈ alkoxyl or —NRₘRₙ, wherein each occurrence of Rₘ or Rₙ are independently hydrogen, hydroxyl, unsubstituted or substituted C₁₋₈ alkyl, or unsubstituted or substituted C₁₋₈ alkoxyl;

wherein said cycloalkyl, cycloalkenyl, heterocycle, aryl or heteroaryl may optionally attach via a C₁₋₈ alkyl or C₁₋₈ alkoxyl linker;

or a pharmaceutically acceptable salt, prodrug, tautomer, regioisomer, stereoisomer, diastereomer, enantiomer or racemate thereof;

with the proviso that when R₂, R₃, R₄ and R₇ are each hydrogen, R₁ is hydroxyl and R₅ is C(═NOH)NH₂, then R₆ is not —NRⁱRⁱⁱ where Rⁱ is hydrogen and Rⁱⁱ is alkenyl substituted alkyl;

further with the proviso that when R₂ is N₃, R₁, R₃, R₄ and R₇ are each hydrogen, and R₆ is NH₂, then R₅ is not C(O)NH₂;

further with the proviso that when R₂ is methyl, R₁ is hydroxyl, R₃, R₄ and R₇ are each hydrogen, and R₅ is CN; then R₆ is not NH₂;

further with the proviso that when R₂, R₃, R₄ and R₇ are each hydrogen, R₁ is hydroxyl and R₅ is C(S)NH₂, then R₆ is not NH₂;

when R_{c}, Rₑ, R_{f} or R_{g} is absent, the corresponding O atom is negatively charged and a counterion is present, and when R_{d} is absent the corresponding N atom is protonated and a counterion is present.

According to another aspect of the present invention, compounds of Formula II are provided herein:

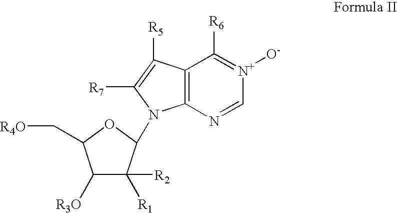

wherein:

R₁ and R₂ are independently selected from the group consisting of halogen, hydrogen, hydroxyl, N₃, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl and —NR′R″, wherein each occurrence of R′ and R″ are independently selected from the group consisting of hydrogen, hydroxyl, unsubstituted or substituted C₁₋₈ alkyl, unsubstituted or substituted C₁₋₈ alkoxyl, and unsubstituted or substituted C₃₋₆ cycloalkyl;

R₁ and R₂ are independently ORₓ or OR_{y}; or

R₁ forms an unsubstituted or substituted 5-7 member ring with R₃ wherein said ring optionally comprises 1-2 additional heteroatoms selected from N, O or S; and

R₃, R₄, Rₓ and R_{y} are independently selected from the group consisting of (a) hydrogen,

(b) unsubstituted or substituted C₁₋₈ alkyl, —C(═O)—Rₐ, —C(═O)—ORₐ or —C(═O)—NRₐRₐ′ wherein each occurrence of Rₐ and Rₐ′ are independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S;

(c) monophosphate, diphosphate or triphosphate,

(d) a moiety of Formula A, A′ or A″:

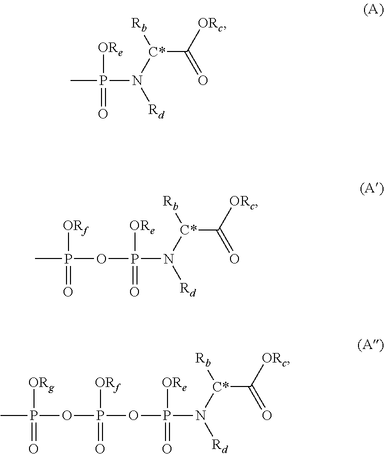

wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,

wherein R_{z} is hydrogen or unsubstituted or substituted C₁₋₈ alkyl;

R_{c}, R_{d}, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,

Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,

R_{b} R_{d}, C* and N may form an unsubstituted or substituted 4-6 membered heterocycle comprising 1-3 additional heteroatoms selected from N, O or S;

(e) an amino acyl moiety of an amino acid; or

(f) a moiety of Formula B, B′ or B″;

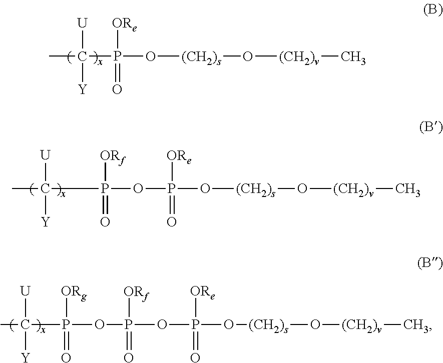

wherein U and Y are independently H or halogen, x is 0, 1 or 2, s is an integer from 2 to 6, v is an integer from 11 to 25, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S, and Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S; or

R₃ and R₄ form a 5′,3′-cyclic phosphate as shown in Formula E′:

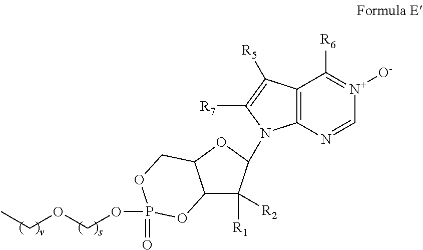

wherein s is an integer from 2 to 6, v is an integer from 11 to 25 and R₁, R₂, R₅, R₆ and R₇ are as described herein; and

R⁵, R⁶ and R⁷ are independently selected from the group consisting of hydrogen, halogen, hydroxyl, CN, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl, unsubstituted or substituted C₁₋₈ thioalkyl, unsubstituted or substituted C₃₋₆ cycloalkyl, unsubstituted or substituted C₃₋₆ cycloalkenyl, unsubstituted or substituted C₆₋₁₄ aryl, unsubstituted or substituted heteroaryl comprising 1-4 heteroatoms selected from N, O and S, and —NRⁱRⁱⁱ, wherein at each occurrence Rⁱ and Rⁱⁱ are independently selected from the group consisting of hydrogen, hydroxyl, unsubstituted or substituted C₁₋₈ alkyl, unsubstituted or substituted C₁₋₈ alkenyl, unsubstituted or substituted C₁₋₈ alkynyl, unsubstituted or substituted C₁₋₈ alkoxyl and unsubstituted or substituted C₃₋₆ cycloalkyl, or

R₅, R₆ and R₇ are independently Formula C:

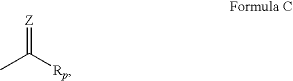

wherein Z is selected from the group consisting of O, S and NRⱼ, wherein Rⱼ is hydrogen, hydroxyl or unsubstituted or substituted C₁₋₈ alkoxyl; Rₚ is hydrogen, unsubstituted or substituted C₁₋₈ alkoxyl or NRₘRₙ, wherein each occurrence of Rₘ or Rₙ are independently hydrogen, hydroxyl, unsubstituted or substituted C₁₋₈ alkyl, or unsubstituted or substituted C₁₋₈ alkoxyl;

wherein said cycloalkyl, cycloalkenyl, heterocycle, aryl or heteroaryl may optionally attach via a C₁₋₈ alkyl or C₁₋₈ alkoxyl linker;

or a pharmaceutically acceptable salt, prodrug, tautomer, regioisomer, stereoisomer, diastereomer, enantiomer or racemate thereof;

when R_{c}, Rₑ, R_{f} or R_{g} is absent, the corresponding O atom is negatively charged and a counterion is present, and when R_{d} is absent the corresponding N atom is protonated and a counterion is present.

In some embodiments, R₁ and R₂ are independently F or methyl, or R₁ and R₂ are independently hydrogen, methyl or hydroxyl.

In another embodiment, at least one of R₃ or R₄ is hydrogen. In one embodiment, R₃ is hydrogen. In one embodiment, R₄ is selected from the group consisting of monophosphate, diphosphate and triphosphate. Yet, in some embodiments, R₃ and R₄ are independently selected from the group consisting of —C(═O)—Rₐ, —C(═O)—ORₐ and —C(═O)—NRₐRₐ′ wherein each occurrence of Rₐ and Rₐ′ are independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S. In another embodiment, R₃ and R₄ are independently an amino acyl moiety of an amino acid.

Yet, in other embodiments, R₃ and R₄ are independently a moiety of Formula A, A′ or A″:

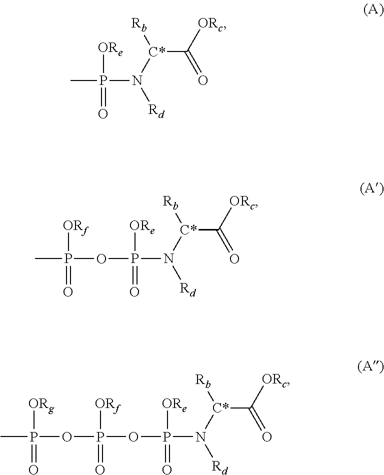

wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,

wherein R_{z} is hydrogen or unsubstituted or substituted C₁₋₈ alkyl;

R_{c}, R_{d}, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and, wherein R_{b} R_{d}, C* and N may form an unsubstituted or substituted 4-6 membered heterocycle comprising 1-3 additional heteroatoms selected from N, O or S, and Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

In some embodiments, R₃ or R₄ is independently an α-amino acyl moiety of a natural α-amino acid as defined herein.

In some embodiments, R₃ or R₄ is independently Formula D:

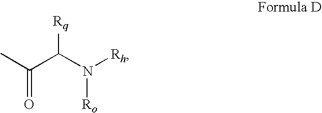

wherein

R_{q} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OH, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S;

Rₒ and Rₕ are independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl, unsubstituted or substituted heteroaryl comprising 1-4 heteroatoms selected from N, O and S, and —C(═O)—Rₖ, wherein Rₖ is selected from the group consisting of unsubstituted or substituted C₁₋₈ alkoxyl, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

In one embodiment, R₃ and R₄ are independently a moiety of Formula B, B′ or

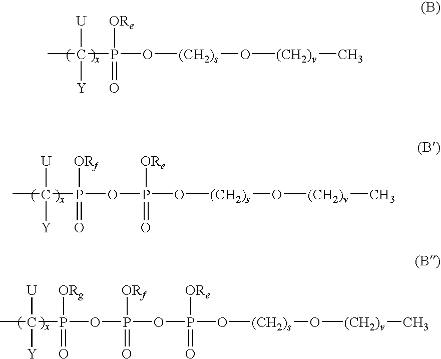

wherein U and Y are independently H or halogen, x is 0, 1 or 2, s is an integer from 2 to 6, v is an integer from 11 to 25, and Rₑ, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S. In some embodiments, U and Y are independently H or F. In one embodiment, x is 0. In other embodiment, x is 1. In some embodiments, s is an integer from 2 to 4 and v is an integer from 11 to 23. In another embodiment, s is an integer from 2 or 3 and v is an integer from 14 to 18. In another embodiment, s is 3 and v is 15.

In another embodiment, R₃ and R₄ form a 5′,3′-cyclic phosphate as shown in

Formula E:

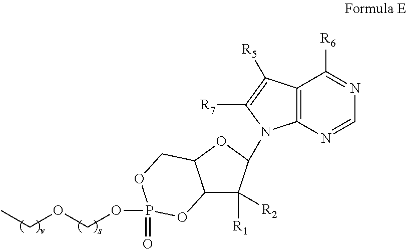

wherein s is an integer from 2 to 6, v is an integer from 11 to 25 and R₁, R₂, R₅, R₆ and R₇ are as described herein.

In another embodiment, R₃ and R₄ form a 5′,3′-cyclic phosphate as shown in Formula E′:

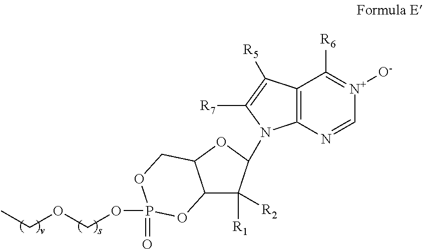

wherein s is an integer from 2 to 6, v is an integer from 11 to 25 and R₁, R₂, R₅, R₆ and R₇ are as described herein.

In some embodiments, R₆ is hydrogen or NH₂. In some embodiments, R₆ is hydrogen, unsubstituted or substituted C₁₋₈ alkoxyl, unsubstituted or substituted C₁₋₈ thioalkyl, or NRⁱRⁱⁱ, wherein each occurrence of Rⁱ and Rⁱⁱ are independently selected from the group consisting of hydrogen, hydroxyl, unsubstituted or substituted C₁₋₈ alkyl, unsubstituted or substituted C₁₋₈ alkenyl, unsubstituted or substituted C₁₋₈ alkynyl, unsubstituted or substituted C₁₋₈ alkoxyl and unsubstituted or substituted C₃₋₆ cycloalkyl.

In another embodiment, at least one of R₅, R₆ or R₇ is halogen.

In another embodiment, R₂ is methyl, R₁ is F, and R₃ and R₄ are each hydrogen.

In another embodiment, R₂ is methyl and R₁, R₃ and R₄ are each hydrogen.

In another embodiment, R₁, R₂, R₃ and R₄ are each hydrogen.

In another embodiment, R₄ is a moiety of formula B:

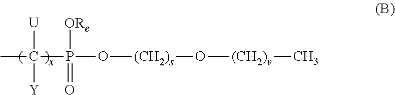

wherein U and Y are independently H or halogen, x is 0, 1 or 2, s is an integer from 2 to 6, v is an integer from 11 to 25, and Rₑ is absent or selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

In another embodiment, R₄ is a moiety of formula B:

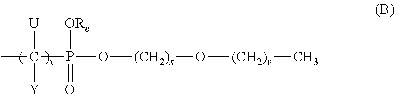

where x is 0, s is 3, v is 15 and Rₑ is absent, hydrogen, unsubstituted or substituted C₁₋₈ alkyl or unsubstituted or substituted C₆₋₁₄ aryl.

In another embodiment, R₄ is a moiety of formula B:

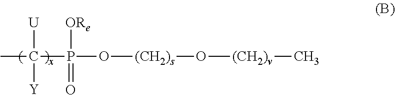

where x is 0, s is 2-4, v is 11-20 and Rₑ is absent, hydrogen, unsubstituted or substituted C₁₋₈ alkyl or unsubstituted or substituted C₆₋₁₄ aryl.

In another embodiment, R₃, R₄, Rₓ or R_{y} is a moiety of formula B:

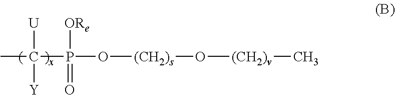

where x is 0-2, s is 2-4, v is 11-20 and Rₑ is (CH₂)ₛ—O—(CH₂)ᵥ—CH₃.

In another embodiment, R₅ is C(S)NH₂, R₆ is NH₂ and R₃ and R₄ form a 5′,3′-cyclic phosphate as shown in Formula E:

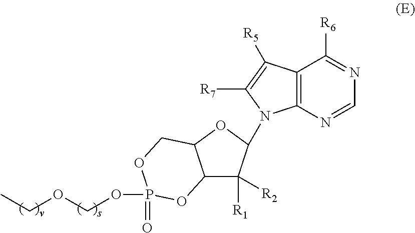

wherein s is an integer from 2 to 6, v is an integer from 11 to 25 and R₅, R₆ and R₇ are as defined herein.

In another embodiment, R₄ is a moiety of formula Aₐ:

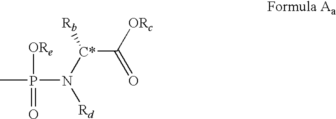

wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,

wherein R_{Z} is hydrogen or unsubstituted or substituted C₁₋₈ alkyl;

and R_{c}, R_{d}, and Rₑ are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S, wherein R_{b} R_{d}, C* and N may form an unsubstituted or substituted 4-6 membered heterocycle comprising 1-3 additional heteroatoms selected from N, O or S.

In another embodiment, R₄ is a moiety of formula Aₐ:

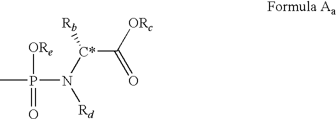

wherein R_{b} is methyl, Rₑ is phenyl, R_{d} is hydrogen, and R_{c} is methyl.

In another embodiment, R₅ is halogen.

In another embodiment, R₅ is cyano.

In another embodiment, R₅ is Formula C:

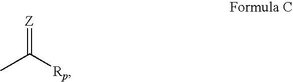

wherein Z is selected from the group consisting of O, S, and NRⱼ, wherein Rⱼ is hydrogen, hydroxyl or unsubstituted or substituted C₁₋₈ alkoxyl; Rₚ is hydrogen, unsubstituted or substituted C₁₋₈ alkoxyl or —NRₘRₙ, wherein each occurrence of Rₘ or Rₙ are independently selected from the group consisting of hydrogen, hydroxyl, unsubstituted or substituted C₁₋₈ alkyl and unsubstituted or substituted C₁₋₈ alkoxyl.

In another embodiment, R₅ is C(═N—OH)NH₂.

In another embodiment, R₅ is C(O)NH₂.

In another embodiment, R₅ is Br.

In another embodiment, R₅ is C(═NH)OCH₃.

In another embodiment, R₅ is C(S)NH₂.

In another embodiment, R₅ is unsubstituted or substituted thiophenyl.

In another embodiment, R₅ is C₁₋₈ alkylaminocarbonyl substituted thiophenyl.

In another embodiment, R₅ is unsubstituted thiophenyl.

In another embodiment, R₅ is C(═NH)NHOH.

In another embodiment, R₆ is C₂₋₈ alkenyl substituted amine.

In another embodiment, R₆ is NH₂.

In another embodiment, R₆ is C₁₋₈ alkyl substituted amine.

In another embodiment, R₄ is a monophosphate, diphosphate or triphosphate.

In another embodiment, R₄ is a monophosphate.

In another embodiment, R₄ is a triphosphate.

In another embodiment, R₄ is a moiety of formula Aₐ:

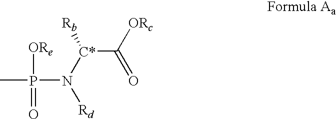

wherein Rₑ is absent, forming an O⁻ moiety where a counterion is present (e.g., Li⁺, Na⁺, NH₄⁺, etc.), and further wherein R_{b}, R_{c} and R_{d} are as defined above.

In another embodiment, R₄ is a moiety of formula Aₐ:

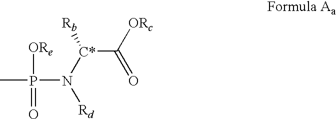

wherein R_{d} is absent, forming an N⁺ moiety which is bound to a pharmaceutically acceptable anion, and further wherein R_{b}, R_{c} and Rₑ are as defined above.

In another embodiment, one of Rₓ, R₃ and R₄ is —C(═O)—ORₐ or —C(═O)—NRₐRₐ′ wherein each occurrence of Ra and Ra′ is independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

In another embodiment, two of Rₓ, R₃ and R₄ are —C(═O)—ORₐ or —C(═O)—NRₐRₐ′ wherein each occurrence of Ra and Ra′ is independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

In another embodiment, each of Rₓ, R₃ and R₄ is —C(═O)—ORₐ or —C(═O)—NRₐRₐ′ wherein each occurrence of Ra and Ra' is independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

In another embodiment, R₄ is:

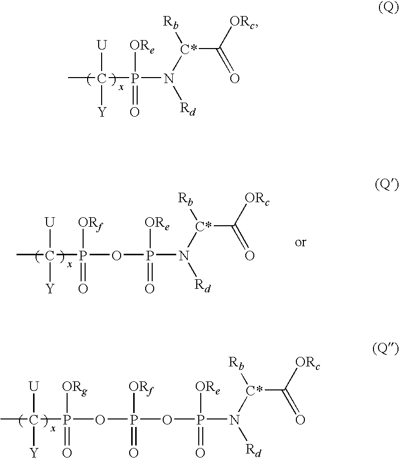

wherein U and Y are independently hydrogen or halogen, x is 1 or 2, R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,

wherein R_{z} is hydrogen or unsubstituted or substituted C₁₋₈ alkyl;

and R_{c}, R_{d}, Rₑ, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₄ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S, wherein R_{b} R_{d}, C* and N may form an unsubstituted or substituted 4-6 membered heterocycle comprising 1-3 additional heteroatoms selected from N, O or S.

In a particular embodiment, compounds of the present invention have the structure of Formula Ix, Formula Iy or Formula Iz:

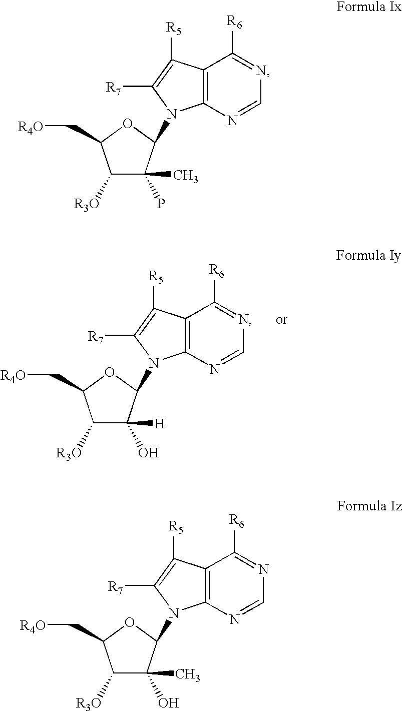

In another embodiment, the compounds of present invention have the structure of Formula IIx, IIy or IIz:

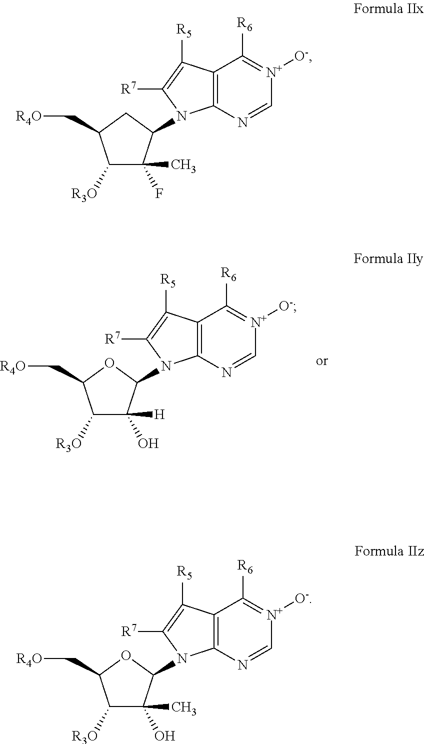

Yet, in a particular embodiment, the compounds of present invention have the structure of:

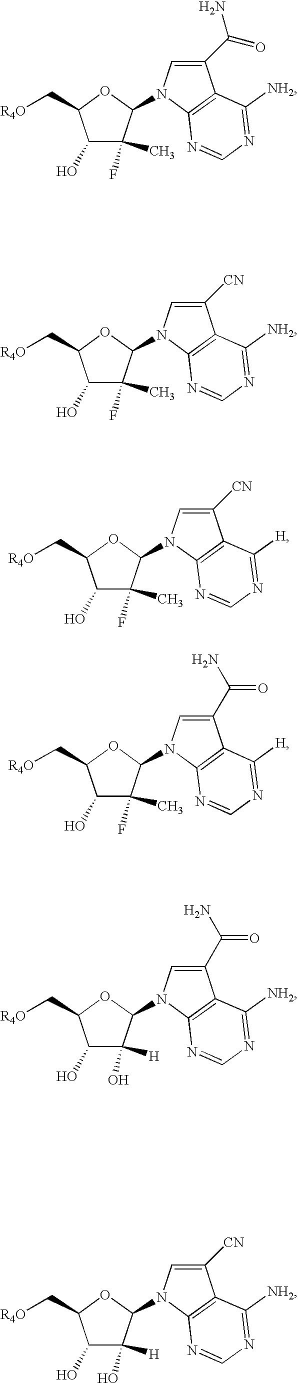

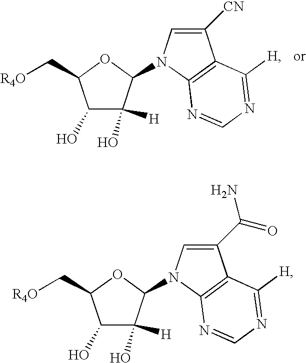

wherein R₄ is selected from the group consisting of hydrogen, monophosphate, diphosphate and triphosphate; or R₄ is a moiety of Formula B;

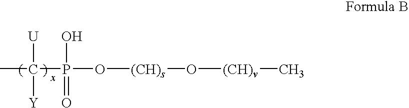

wherein U and Y are independently H or halogen, x is 0, 1 or 2, s is an integer from 2 to 6 and v is an integer from 11 to 25.

Yet, in a further embodiment, the compounds of the present invention have the structure of:

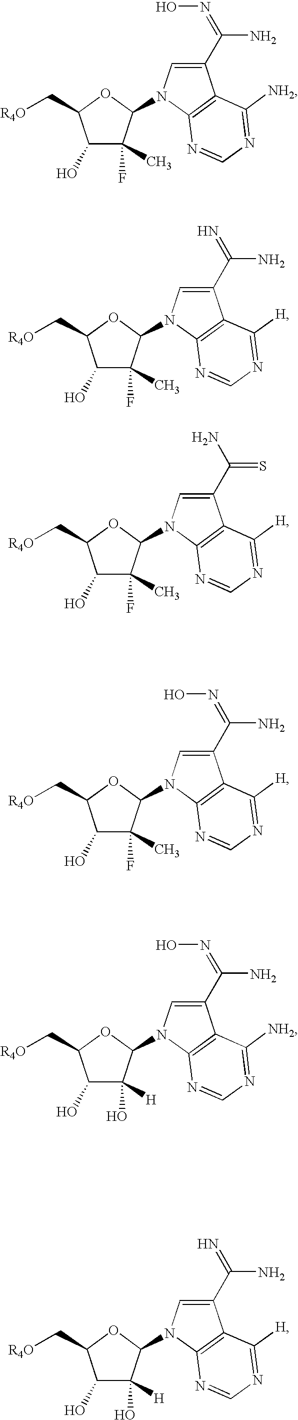

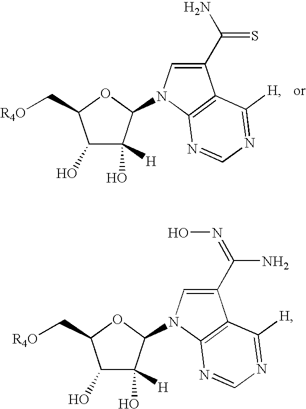

wherein R₄ is selected from the group consisting of hydrogen, monophosphate, diphosphate and triphosphate; or R₄ is a moiety of Formula B;

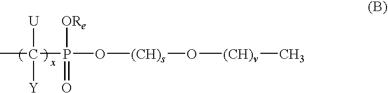

wherein U and Y are independently H or halogen, x is 0, 1 or 2, s is an integer from 2 to 6 and v is an integer from 11 to 25.

In one embodiment, compound of Formula II have the structure of:

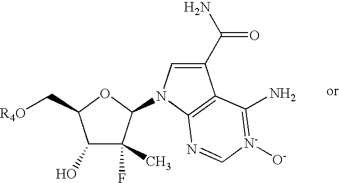

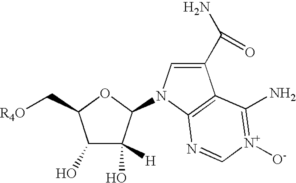

wherein R₄ is selected from the group consisting of hydrogen, monophosphate, diphosphate and triphosphate; or R₄ is a moiety of Formula B;

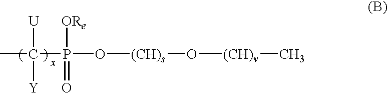

wherein U and Y is independently H or halogen, x is 0, 1 or 2, s is an integer from 2 to 6 and v is an integer from 11 to 25.

In another embodiment, the present invention provides compounds of Formula I:

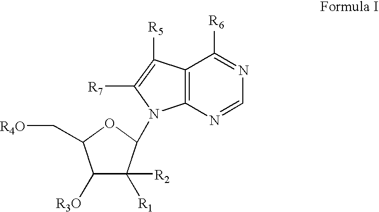

wherein:

R₁ is hydrogen or unsubstituted or substituted C₁₋₈ alkyl;

R₂ is halogen or hydroxyl;

R₃ is hydrogen and R₄ is:
(a) hydrogen;
(b) a moiety of Formula A:

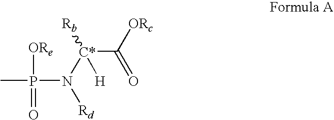

wherein R_{b} is selected from the group consisting of hydrogen, unsubstituted or substituted C₁₋₈ alkyl, unsubstituted or substituted C₂₋₈ alkenyl, or unsubstituted or substituted C₂₋₈ alkynyl, and R_{c}, R_{d}, and Rₑ are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, or unsubstituted or substituted C₆₋₁₄ aryl; or
(c) a moiety of Formula B;

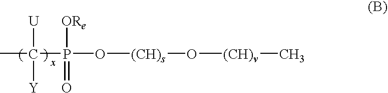

wherein U and Y are independently H or halogen, x is 0, 1 or 2, s is an integer from 2 to 6 and v is an integer from 11 to 25; or
R₃ and R₄ form a 5′,3′-cyclic phosphate as shown in Formula E:

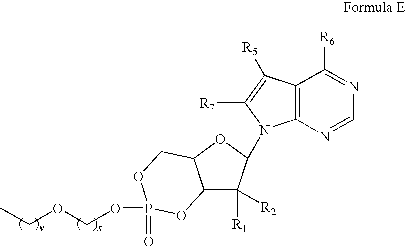

wherein s is an integer from 2 to 6 and v is an integer from 11 to 25;

R₅ is halogen, unsubstituted or substituted heteroaryl comprising 1-4 heteroatoms selected from N, O and S, cyano, or a moiety of Formula C:

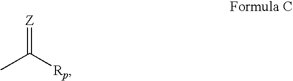

wherein Z is selected from the group consisting of O, S and NRⱼ, wherein Rⱼ is hydrogen; Rₚ is hydrogen, unsubstituted or substituted C₁₋₈ alkoxyl or —NRₘRₙ, wherein each occurrence of Rₘ or Rₙ is independently hydrogen, hydroxyl, unsubstituted or substituted C₁₋₈ alkyl, or unsubstituted or substituted C₁₋₈ alkoxyl;

R₆ is —NRⁱRⁱⁱ, wherein at each occurrence Rⁱ and Rⁱⁱ are independently selected from the group consisting of hydrogen, hydroxyl, unsubstituted or substituted C₁₋₈ alkyl, unsubstituted or substituted C₁₋₈ alkoxyl and unsubstituted or substituted C₃₋₆ cycloalkyl; and

R₇ is hydrogen, unsubstituted or substituted C₁₋₈ alkyl, unsubstituted or substituted C₂₋₈ alkenyl, or unsubstituted or substituted C₂₋₈ alkynyl;

or a pharmaceutically acceptable salt, prodrug, tautomer, regioisomer, stereoisomer, diastereomer, enantiomer or racemate thereof;

with the proviso that when R₅ is cyano R₄ is not hydrogen;

further with the proviso that when R₅ is halogen R₂ is halogen;

and further with the proviso that when R₅ is —C(S)NH₂ and R₁ is hydrogen, R₄ is not hydrogen.

In a particular embodiment, the present invention includes one or more compounds listed in Table A or a pharmaceutically acceptable salt, prodrug, tautomer, regioisomer, stereoisomer, diastereomer, enantiomer or racemate thereof

TABLE A

| | |
|---|---|
| Compound a | 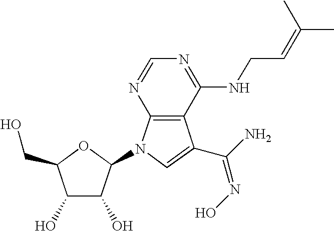 |
| Compound b | 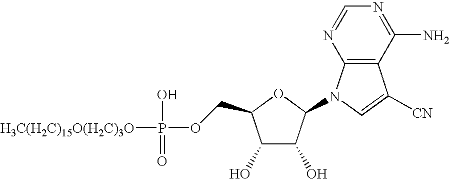 |
| Compound c | 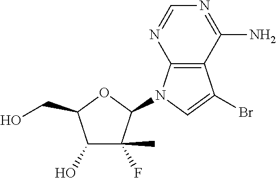 |
| Compound d | 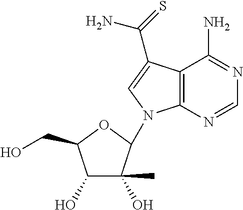 |
| Compound e | 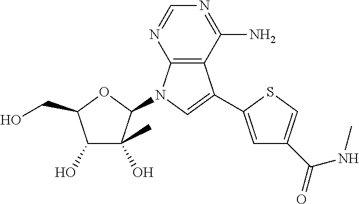 |
| Compound f | 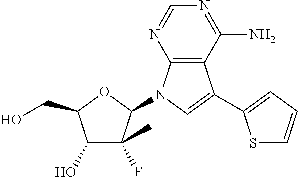 |
| Compound g | 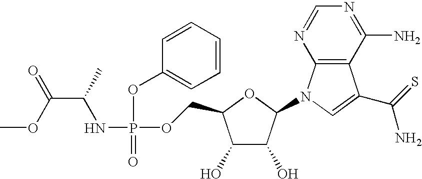 |
| Compound h | 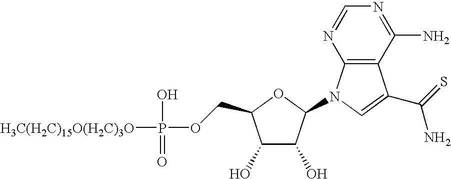 |
| Compound j | 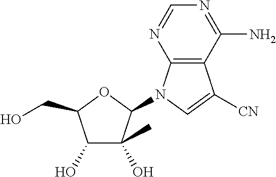 |
| Compound k | 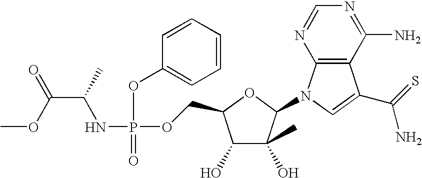 |
| Compound l | 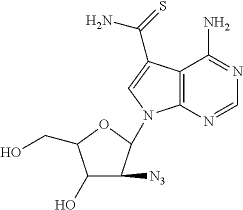 |
| Compound m | 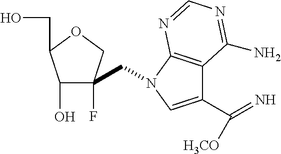 |
| Compound n | 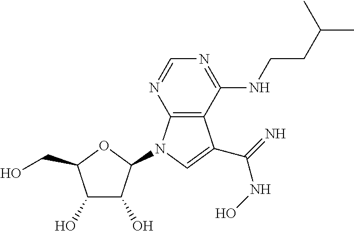 |
| Compound o | 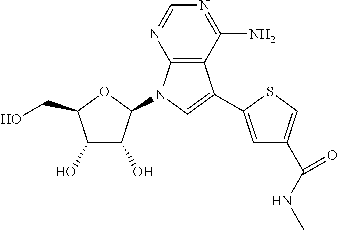 |
| Compound p | 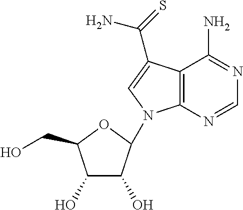 |
| Compound q | 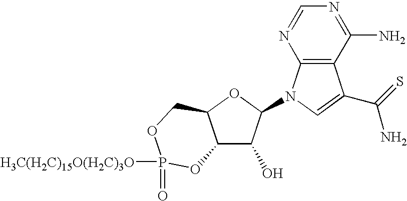 |
| Compound r | 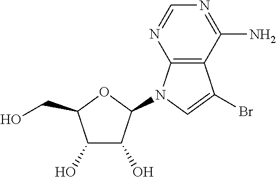 |

In another embodiment, the compounds of the present invention exhibit an EC₅₀ of less than 5 μM against a virus (e.g., HCV). For example, a compound of Formula I, II or Compound a, b, c, d, e, f, g, h, j, k, l, m, n, o, p, q or r exhibits an EC₅₀ of less than 5 μM. For example, a compound of Formula I, II or Compound a, b, c, d, e, f, g, h, j, k, l, m, n, o, p, q or r exhibits an EC₅₀ of less than 1 μM. For example, a compound of Formula I, II or Compound a, b, c, d, e, f, g, h, j, k, l, m, n, o, p, q or r exhibits an EC₅₀ of less than 0.1 μM. For example, a compound of Formula I, II or Compound a, b, c, d, e, f, g, h, j, k, l, m, n, o, p, q or r exhibits an EC₅₀ of less than 0.01 μM. For example, a compound of Formula I, II or Compound a, b, c, d, e, f, g, h, j, k, l, m, n, o, p, q or r exhibits an EC₅₀ of less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or 0.005 μM. For example, a compound of Formula I, II or Compound a, b, c, d, e, f, g, h, j, k, l, m, n, o, p, q or r exhibits an EC₅₀ of less than 5, 4, 3, 2, 1, or 0.5 μM.

In another embodiment, the compounds of the present invention exhibit a CC₅₀ of greater than 1 μM. For example, a compound of Formula I, II or Compound a, b, c, d, e, f, g, h, j, k, l, m, n, o, p, q or r exhibits a CC₅₀ of greater than 1 μM. For example, a compound of Formula I, II or Compound a, b, c, d, e, f, g, h, j, k, l, m, n, o, p, q or r exhibits a CC₅₀ of greater than 20 μM. For example, a compound of Formula I, II or Compound a, b, c, d, e, f, g, h, j, k, l, m, n, o, p, q or r exhibits a CC₅₀ of greater than 50 μM. For example, a compound of Formula I, II or Compound a, b, c, d, e, f, g, h, j, k, l, m, n, o, p, q or r exhibits a CC₅₀ of greater than 100 μM. For example, a compound of Formula I, II or Compound a, b, c, d, e, f, g, h, j, k, l, m, n, o, p, q or r exhibits a CC₅₀ of greater than 1, 2, 3, 4, 5, 6, 7, 8, 9, 10, 15, 20, 25, 30, 35, 40, 45, 50, 55, 60, 65, 70, 75, 80, 85, 90, 95 or 100 μM. For example, a compound of Formula I, II or Compound a, b, c, d, e, f, g, h, j, k, l, m, n, o, p, q or r exhibits a CC₅₀ of greater than 100, 110, 120 or 130 μM.

In another embodiment, the compounds of the present invention exhibit a TC₅₀ (MT-4) of greater than 1 μM. For example, a compound of Formula I, II or Compound a, b, c, d, e, f, g, h, j, k, l, m, n, o, p, q or r exhibits a TC₅₀ of greater than 1 μM. For example, a compound of Formula I, II or Compound a, b, c, d, e, f, g, h, j, k, l, m, n, o, p, q or r exhibits a TC₅₀ of greater than 20 μM. For example, a compound of Formula I, II or Compound a, b, c, d, e, f, g, h, j, k, l, m, n, o, p, q or r exhibits a TC₅₀ of greater than 50 μM. For example, a compound of Formula I, II or Compound a, b, c, d, e, f, g, h, j, k, l, m, n, o, p, q or r exhibits a TC₅₀ of greater than 100 μM. For example, a compound of Formula I, II or Compound a, b, c, d, e, f, g, h, j, k, l, m, n, o, p, q or r exhibits a TC₅₀ of greater than 1, 2, 3, 4, 5, 6, 7, 8, 9, 10, 15, 20, 25, 30, 35, 40, 45, 50, 55, 60, 65, 70, 75, 80, 85, 90, 95 or 100 μM. For example, a compound of Formula I, II or Compound a, b, c, d, e, f, g, h, j, k, l, m, n, o, p, q or r exhibits a TC₅₀ of greater than 100, 110, 120 or 130 μM.

In another embodiment, the compounds of the present invention exhibit a mitotoxicity of greater than 20 μM. For example, a compound of Formula I, II or Compound a, b, c, d, e, f, g, h, j, k, l, m, n, o, p, q or r exhibits a mitotoxicity of greater than 50 μM. For example, a compound of Formula I, II or Compound a, b, c, d, e, f, g, h, j, k, l, m, n, o, p, q or r exhibits a mitotoxicity of greater than 80 μM. For example, a compound of Formula I, II or Compound a, b, c, d, e, f, g, h, j, k, l, m, n, o, p, q or r exhibits a mitotoxicity of greater than 90 μM. For example, a compound of Formula I, II or Compound a, b, c, d, e, f, g, h, j, k, l, m, n, o, p, q or r exhibits a mitotoxicity of greater than 15, 20, 25, 30, 35, 40, 45, 50, 55, 60, 65, 70, 75, 80, 85 or 90 μM.

The compounds described herein may contain one or more asymmetric centers, depending upon the location and nature of the various substituents. Asymmetric carbon atoms may be present in the (R) or (S) configuration. When the orientation of a bond around a chiral center is not specified in a formula, it is to be understood that the formula encompasses every possible isomer such as geometric isomer, optical isomer, stereoisomer and tautomer based on asymmetric carbon, which can occur in the structures of the compounds described herein. In one embodiment, the compounds of the present invention are isomers with the configuration which produces the compound described herein with the more desirable biological activity. In certain embodiments, asymmetry may also be present due to restricted rotation about a given bond, for example, the central bond adjoining two aromatic rings of the specified compounds. Substituents on a ring may also be present as either cis or trans isomer and a substituent on a double bond may be present in either Z or E isomer. It is intended that all isomers (including enantiomers and diastereomers), either by nature of asymmetric centers or by restricted rotation as described above, as separated, pure or partially purified isomers or racemic mixtures thereof, be included within the scope of the present invention. The purification of said isomers and the separation of said isomeric mixtures may be accomplished by standard techniques known in the art.

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

The present invention includes salts of the compounds of Formulae I, II and Compounds a, b, c, d, e, f, g, h, j, k, l, m, n, o, p, q and r. For example, R_{c}, R_{d}, Rₑ, R_{f} and/or R_{g} can be absent, which results in the formation of the corresponding ion (e.g., O⁻) or N is protonated. Such an ion can be associated with, e.g., non-covalently, physiologically acceptable anions (e.g., tosylate, methanesulfonate, acetate, citrate, malonate, tartarate, succinate, benzoate, ascorbate, α-ketoglutarate, α-glycerophosphate, sulfate, nitrate, bicarbonate, or carbonate) or physiologically acceptable cations (e.g., sodium, potassium, lithium) known in the art.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the compounds described herein may be administered as a nucleoside prodrug to increase the activity, bioavailability, stability or otherwise alter the properties of the nucleoside. A number of nucleoside prodrug ligands are known. In general, alkylation, acylation or other lipophilic modification of the mono, di or triphosphate of the nucleoside will increase the stability of the nucleoside. Examples of substituent groups that can replace one or more hydrogens on the phosphate moiety are alkyl, aryl, steroids, carbohydrates, including sugars, 1, 2-diacylglycerol and alcohols. Many are described in R. Jones and N. Bischofberger, Antiviral Research, 27 (1995) 1-17. Any of these can be used in combination with the disclosed nucleosides to achieve a desired effect.

The active compounds described herein can also be provided as 5′-phosphoether lipids or 5′-ether lipids, as disclosed in the following references, which are incorporated by reference herein: Kucera, et al, Novel membrane-interactive ether lipid analogs that inhibit infectious HIV-1 production and induce defective virus formation, AIDS Res. Hum. RetroViruses, vol. 6, 491-501 (1990); Piantadosi et al., Synthesis and evaluation of novel ether lipid nucleoside conjugates for anti-HIV activity, J. Med. Chem. Vol. 34, 1408-1414 (1991); Hostetler et al., Greatly enhanced inhibition of human immunodeficiency virus type 1 replication in CEM and HT4-6C cells by 3′-deoxythymidine diphosphate dimyristoylglycerol, a lipid prodrug of 3′-deoxythymidine, Antimicrob. Agents Chemother, vol. 36, 2025-2029 (1992).

Nonlimiting examples of U.S. patents that disclose suitable lipophilic substituents that can be covalently incorporated into the nucleoside, preferably at the 5′-OH position of the nucleoside or lipophilic preparations, include U.S. Pat. Nos. 5,149,794; 5,194,654; 5,223,263; 5,256,641; 5,411,947; 5,463,092; 5,543,389; 5,543,390; 5,543,391, and 5,554,728, each of which is incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0,350,287, EP 0,650,371, and WO 91/19721. In some embodiments of the present invention, the 5′-OH position corresponds to the “—OR₄” in formulas of compounds described herein wherein R₄ is H.

### C. PHARMACEUTICAL COMPOSITIONS AND ADMINISTRATION

In one embodiment, the present invention is a pharmaceutical composition comprising the compounds described herein. In another embodiment, the pharmaceutical composition further comprises a pharmaceutically acceptable carrier. The term “pharmaceutically acceptable carrier” as used herein refers to any substance, not itself a therapeutic agent, used as a vehicle for delivery of a therapeutic agent to a subject. In some embodiments, the pharmaceutical composition further comprises one or more additional therapeutically active agents against HCV described in Section D.

Any suitable route of administration may be employed for providing a mammal, especially a human with an effective dosage of a compound of the present invention. For example, the compositions of the present invention may be suitable for formulation for oral, parenteral, inhalation spray, topical, rectal, nasal, sublingual, buccal, vaginal or implanted reservoir administration, etc. In some embodiments, the compositions are administered orally, topically, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

A pharmaceutically acceptable oil may be employed as a solvent or suspending medium in compositions of the present invention. Fatty acids, such as oleic acid and its glyceride derivatives are suitably included in injectable formulations, as are natural pharmaceutically acceptable oils, such as olive oil or castor oil, especially in their polyoxyethylated versions. The oil containing compositions of the present invention may also contain a long-chain alcohol diluent or dispersant, such as carboxymethyl cellulose or similar dispersing agents that are commonly used in the formulation of pharmaceutically acceptable dosage forms including emulsions and suspensions. The compositions suitably further comprise surfactants (such as non-ionic detergents including Tween® or Span®) other emulsifying agents, or bioavailability enhancers.

The compositions of this invention may be in the form of an orally acceptable dosage form including, but not limited to, capsules, tablets, suspensions or solutions. The oral dosage form may include at least one excipient. Excipients used in oral formulations of the present can include diluents, substances added to mask or counteract a disagreeable taste or odor, flavors, dyes, fragrances, and substances added to improve the appearance of the composition. Some oral dosage forms of the present invention suitably include excipients, such as disintegrants, binding agents, adhesives, wetting agents, polymers, lubricants, or glidants that permit or facilitate formation of a dose unit of the composition into a discrete article such as a capsule or tablet suitable for oral administration. Excipient-containing tablet compositions of the invention can be prepared by any suitable method of pharmacy which includes the step of bringing into association one or more excipients with a compound of the present invention in a combination of dissolved, suspended, nanoparticulate, microparticulate or controlled-release, slow-release, programmed-release, timed-release, pulse-release, sustained-release or extended-release forms thereof

Alternatively, pharmaceutically acceptable compositions of this invention may be in the form of a suppository for rectal administration. The suppositories can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of the present invention may be in the faun of a topical solution, ointment, or cream in which the active component is suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Where the topical formulation is in the form of an ointment or cream, suitable carriers include, but are not limited to, mineral oil, sorbitan monostearate, polysorbate 60, cetyl esters wax, cetearyl alcohol, 2 octyldodecanol, benzyl alcohol and water.

The pharmaceutically acceptable compositions of this invention may also be administered by nasal, aerosol or by inhalation administration routes. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Additionally, the pharmaceutical formulation including compounds of the present invention can be in the form of a parenteral formulation. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments, the pharmaceutically compositions of this invention are formulated for oral administration. For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions may be provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

It should also be understood that a specific dosage and treatment regimen for any particular patient will depend upon a variety of factors, including the activity of the specific compound employed, the mode of administration, the age, body weight, general health, gender, diet, rate of excretion, drug combination, and the judgment of the treating physician, the condition being treated and the severity of the condition. Such dosage may be ascertained readily by a person skilled in the art. This dosage regimen may be adjusted to provide the optimal therapeutic response.

Compounds of the present invention may optionally be administered in conjunction with one or more additional active compounds and/or agents useful in the treatment of viral infections as described herein. The additional compound(s) may optionally be administered concurrently. As used herein, the word “concurrently” means sufficiently close in time to produce a combined effect (that is, concurrently may be simultaneously, or it may be two or more events occurring within a short time period before or after each other).

### D. METHODS OF USE AND COMBINATION THERAPY

Another aspect of the present invention provides methods of preventing or treating viral infection in a subject. In some embodiments, the present invention provides methods of preventing or treating Flaviviridae virus infection, for example, Hepatitis C virus (HCV) infection. The methods comprise administering a subject a therapeutically effective amount of a compound described herein.

As used herein, the viral infection includes both (+) Strand RNA viruses and (−) Strand RNA viruses. Exemplary viral infection includes, but is not limited to, Flaviviridae virus such as Dengue fever, Japanese encephalitis, Kyasanur Forest disease, Murray Valley encephalitis, St. Louis encephalitis, Tick-borne encephalitis, West Nile encephalitis, Yellow fever, Hepatitis C Virus Infection, BVDV (1); Picornaviridae such as Rhino type 2, Rhino type 14, Polio 3; Togaviridae such as Western equine encephalitis, Venezuelan equine encephalitis; and Paramyxoviridae such as Respiratory syncytial, and Measles.

Furthermore, one skilled in the art will recognize that any antiviral drug or therapy may be used in combination or alternation with any one or more compounds described in the present invention. For example, as noted above, the compositions of the present invention may include the active compounds as described in section B above in combination with one or more (e.g., 1, 2, 3) additional active agents such as described in this section in analogous manner as known in the art, for example US 2006/0003942 to Tung et al. and US 2005/0037018 A1 to Maertens.

Additional antiviral active agents that may be used with the compounds of the present invention in carrying out the present invention include, but are not limited to, nucleoside polymerase inhibitors, non-nucleoside polymerase inhibitors, protease inhibitors, NS4A inhibitors, immunomodulators, cyclophilin inhibitors, NS3 helicase inhibitors and a-glucosidase I inhibitors.

In another embodiment, the additional antiviral agents include, but are not limited to, antiviral agent selected from the following table:

TABLE 1
Exemplary additional antiviral agent/compounds

| Compound | Aka | Company | Mechanism |
|---|---|---|---|
| Celgosivir | MX-3253 | Migenix | a-glucosidase I inhibitor |
| Debio 025 | | Debiopharm | cyclophilin inhibitor |
| SCY-635 | | Scynexis | cyclophilin inhibitor |
| Peginterferon alpha | Pegasys | Roche | immunomodulator |
| 2a | | | |
| Peginterferon alpha | Peg-Intron | Schering | immunomodulator |
| 2b | | | |
| interferon 2a | Intron A, | Schering, Roche, others | immunomodulator |
| | Roferon A, etc. | | |
| ANA773 | | Anadys | immunomodulator |
| Nitazoxanide | Alinia, Annita | | immunomodulator |
| GS-9190 | | Gilead | non-nucleoside polymerase |
| VCH-759 | | ViroPharma, Vertex | non-nucleoside polymerase |
| VCH-222 | | ViroPharma, vertex | non-nucleoside polymerase |
| HCV-796 | | ViroPharma, Wyeth | non-nucleoside polymerase |
| ANA598 | | Anadys | non-nucleoside polymerase |
| PF-00868554 | | Pfizer | non-nucleoside polymerase |
| IDX375 | | Idenix | non-nucleoside polymerase |
| A-837093 | | Abbott | non-nucleoside polymerase |
| GSK625433 | | GSK | non-nucleoside polymerase |
| BILN 1941 | | Boehringer Ingelheim | non-nucleoside polymerase |
| ACH 806 | GS-9132 | Achillion/Gilead | NS4A |
| ACH-1095 | GS-9525 | Achillion, Gilead | NS4A |
| MK-06080 | | Merck | Nucleoside Polymerase |
| R7128 | | Pharmasset, Roche | Nucleoside Polymerase |
| R1626 | prodrug of | Roche | Nucleoside Polymerase |
| | R1479 | | |
| Valopicitabine | NM283 | Indenix | Nucleoside Polymerase |
| IDX184 | | Idenix | Nucleoside Polymerase |
| | | | monophosphate prodrug - |
| | | | liver targeted |
| MK-7009 | | Merck | protease inhibitor |
| Boceprevir | SCH 503034 | Schering | protease inhibitor |
| BI 201335 | | Boehringer Ingelheim | protease inhibitor |
| Telaprevir | VX-950 | Vertex | protease inhibitor |
| ITMN-191 | R7227 | InterMune, Roche | protease inhibitor |
| TMC435350 | | Tibotec | protease inhibitor |
| Taribavirin | viramidine | Valeant | ribavirin prodrug |
| Ribavirin | Copegus | Roche | ribavirin |

Exemplary nucleoside polymerase inhibitors are described in U.S. Pat. No. 6,777,395 to Bhat et al., U.S. Pat. No. 7,163,929 to Sommadossi et al. and U.S. Pat. No. 7,202,223 to Roberts et al. Examples of the present invention include, but are not limited to R1626 and IDX184.

Exemplary non-nucleoside polymerase inhibitors are described in U.S. Pat. No. 6,448,281 to Beaulieu et al., U.S. Pat. No. 7,153,880 to Singh et al. and U.S. Pat. No. 6,492,423 to Sergio et al. Examples of the present invention include, but are not limited to ANA598 and VCH-759.

Exemplary protease inhibitors are described in U.S. Patent Publication No. 20090098085 to Sun et al., U.S. Pat. No. 6,995,177 to Bianchi et al. and U.S. Pat. No. 7,273,851 to Miao et al. Examples of the present invention include, but are not limited to Telaprevir and Boceprevir.

Exemplary NS4A inhibitors are described in U.S. Patent Publication No. 20090022688 to Farmer et al. U.S. Pat. No. 7,485,625 to Velazquez et al. and U.S. Pat. No. 7,476,686 to Chen et al. Examples of the present invention include, but are not limited to ACH-1095.

Exemplary immunomodulators are described in U.S. Pat. No. 6,172,046 to Albrecht. Examples of the present invention include, but are not limited to a peginterferon, ribavirin and nitazoxanide.

Exemplary cyclophilin inhibitors are described in U.S. Pat. No. 6,444,643 to Steiner et al., and US Patent Application Publication No. 2007/0275930 to Gentles et al. Examples of the present invention include, but are not limited to Debio 025 and SCY-635.

Exemplary α-glucosidase I inhibitors are described in US Patent Application Publication No. 2008/0019942. Examples of the present invention include, but are not limited to MX-3253.

Additional antiviral/active agents also include, for example, octadecyloxyethyl 9-(S)-[3-methoxy-2-(phosphonomethoxy)propyl]adenine, Pharmasset 7977, and INX-08189.

### Methods of Use and Combination Therapy

Another aspect of the present invention provides methods of preventing or treating influenza infection in a subject. The methods comprise administering a subject a therapeutically effective amount of a compound described herein. The compounds may be used in a monotherapy or combination therapy regime.

As used herein, “monotherapy” refers to the administration of a single active or therapeutic compound to a subject in need thereof. Preferably, monotherapy will involve administration of a therapeutically effective amount of an active compound. For example, influenza monotherapy with one of the compound of the present invention, or a pharmaceutically acceptable salt, prodrug, metabolite, analog or derivative thereof, to a subject in need of treatment of influenza. Monotherapy may be contrasted with combination therapy, in which a combination of multiple active compounds is administered, preferably with each component of the combination present in a therapeutically effective amount. In one aspect, monotherapy with a compound of the present invention, or a pharmaceutically acceptable salt, prodrug, metabolite, polymorph or solvate thereof, is more effective than combination therapy in inducing a desired biological effect.

As used herein, “combination therapy” or “co-therapy” includes the administration of a compound of the present invention, or a pharmaceutically acceptable salt, prodrug, metabolite, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present invention.

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A compound of the present invention, or a pharmaceutically acceptable salt, prodrug, metabolite, analog or derivative thereof, may be administered in combination with a second antiviral compound. For example, as noted above, the compositions of the present invention may include the compounds as described above in combination with one or more (e.g., 1, 2, 3) additional active agents such as described in this section in analogous manner as known in the art.

Additional antiviral active agents that may be used with the compounds of the present invention in carrying out the present invention include, but are not limited to, those that target the M2 ion channel in influenza A viruses (e.g., the adamantanes, such as amantadine and rimantadine); those that inhibit viral uncoating following entry into the cell, agents that block release of the newly formed virions from the surface of infected cells (e.g., the neuraminidase inhibitors, such as oseltamivir and zanamivir).

### E. EVALUATION OF BIOLOGICAL ACTIVITY OF COMPOUNDS

Evaluation of the biological activity of the compounds described herein may be accomplished through in vitro, ex vivo, and in vivo assays that are well known to one skilled in the art. For example:

### (1) Anti-HCV Assays

Antiviral activity against HCV is determined using the stably-expressing HCV replicon cell line, AVA5 (sub-genomic (CON1), genotype 1b) (Okuse, et al., Antivir. Res. 65:23 (2005); Korba, et al., Antivir. Res. 77:56 (2008); Blight, et al., Science 290:1972 (2000)). Reductions in intracellular HCV RNA are determined with respect to a cellular control (B-actin) by blot hybridization. Cytotoxicity is assessed by neutral red dye uptake in parallel plates.

Efficacy and cell cytotoxicity values (EC₅₀, EC₉₀ and CC₅₀) are calculated by linear regression analysis (MS EXCEL®, QuattroPro®) (Korba & Gerin, Antivir. Res. 19:55 (1992); Okuse, et al., Antivir. Res. 65:23 (2005)). The therapeutic index is calculated as CC₅₀/EC₅₀. Recombinant human interferon 2b (PBL laboratories, Inc.) is included as a positive control. Activity is subsequently tested against additional genotypes (e.g., genotype 1a) using the format described for the primary assay. In addition to antiviral assays described herein, several other types of anti-HCV activities can also be assessed using methods known to one of ordinary skill in the art.

### (2) Drug Combinations

Compounds are mixed at approximately equipotent concentrations based on ECₛₒ values and the ratio is maintained during serial dilution (Korba, Antivir. Res. 29:49 (1996)). Typically, 6-8 serial dilutions are tested in the same assay described above for individual drugs. Evaluation of drug interactions is evaluated in comparison to monotherapies using the Combostat® (Biosoft, Inc.) analysis software.

### (3) Drug-Resistant HCV

Since there are currently no licensed anti-HCV drugs for which resistance mutations have been identified, a panel of mutants conferring resistance to compounds in middle to late phase clinical trials has been compiled. This panel will continue to evolve as trials and licensing progress. Stable replicon-containing cell lines that are currently available include genotype 1B NS5B S282T and NS3 A156S and NS3 A156V drug-resistant mutants. (See, Korba, et al., Antivir. Res. 77:56, (2008), Pierra, et al., Nucleosides Nucleotides Nucleic Acids, 24:767 (2005), Courcambeck, et al., Antivir. Ther. 11:847 (2006)). The genetic background is the same as that in the BB7 replicon (AVA5 cells) used in the primary assay. Activity against these mutants is assessed as described in the primary assay, except that semi-quantitative real-time PCR is used for the analysis of HCV RNA due to reduced replication levels.

The following mutants are currently available: NS5B S282T and NS3 R155K. For this assay, Huh7.5 cells are transfected with HCV RNA using Liofectamine 2000™ (Gibco, Inc.) in 6-well culture plates. Three days post-transfection, cultures are exposed to 125 ug/ml G418 and test compounds. After 10-14 days, surviving colonies are fixed, stained, and counted. EC₅₀ and EC₉₀ values are calculated for each transfected RNA.

### (4) Toxicity Evaluations

i. Cytotoxicity Evaluation Using MT-4 Cells

MT-4 cells (human T-cell leukemia) are grown in RPMI1640 medium supplemented with 10% FBS, 2 mM L-glutamine, 100 U/mL penicillin and 100 μg/mL streptomycin. On the day prior to the assay, cells are split to ensure exponential growth during the assay. Cell counts and viability are assessed using a hemocytometer and Trypan Blue dye exclusion. Assays are only conducted if cell viability is greater than 95%. Cells are resuspended at 0.5×10⁴ cells per mL in tissue culture medium and added to the microtiter plates in a 100 μL volume. Compounds to be tested, e.g., candidates are added in a 100 μL volume. They are then incubated at 37° C./5% CO₂ for 6 days prior to staining for cell viability with the tetrazolium dye XTT.

ii. Cytotoxicity Evaluation Using Fresh Human Hepatocytes

Primary human hepatocytes overlay are obtained from XenoTech (Lenexa, Kans., USA). Upon receipt, the medium is replaced with fresh hepatocyte culture medium (XenoTech; catalog #K2300) pre-warmed to 37° C. and the plate is incubated at 37° C./5% CO₂ overnight. Candidate drugs are then added and cells are incubated for 2 days at 37° C. and 5% CO₂ prior to staining with XTT.

iii. XTT Staining for Cell Viability and Compound Cytotoxicity

Cell cytotoxicity (CC₅₀) values are determined by reduction of the tetrazolium dye XTT (2,3-bis(2-methoxy-4-nitro-5-sulfophenyl)-5-[(phenylamino)carbonyl]-2H-tetrazolium hydroxide; Sigma). XTT is metabolized by the mitochondrial enzyme NADPH oxidase to a soluble formazan product in metabolically active cells. XTT solution is prepared daily as a stock of 1 mg/mL in PBS. Phenazine methosulfate (PMS) solution is prepared at 0.15 mg/mL in PBS and stored in the dark at −20° C. XTT/PMS stock is prepared immediately before use by adding 40 μL of PMS per mL of XTT solution. 50 μL of XTT/PMS is added to each well of the plate and the plate incubated for 4 hr at 37° C. The 4 hr incubation has been empirically determined to be within the linear response range for XTT dye reduction with the indicated numbers of cells for each assay. Adhesive plate sealers are used in place of the lids, the sealed plate is inverted to mix the formazan product and the plate read at 450 nm (650 nm reference wavelength) with a Molecular Devices SpectraMax Plus 384 96 well plate format spectrophotometer.

iv. Data Analysis and Evaluation

Microsoft Excel 2003 is used to analyze and graph data. CC₅₀ values are calculated using Microsoft Excel. The CC₅₀ is expressed as mean±standard deviation of triplicate determinations.

### F. SYNTHESIS OF COMPOUNDS

The process to be utilized in the preparation of the compounds described herein depends upon the specific compound desired. Such factors as the selection of the specific substituent and various possible locations of the specific substituent all play a role in the path to be followed in the preparation of the specific compounds of this invention. Those factors are readily recognized by one of ordinary skill in the art.

In general, the compounds of this invention may be prepared by standard techniques known in the art and by known processes analogous thereto. General methods for preparing compounds of the present invention are set forth below. In certain cases, a particular compound is described by way of example as presented further below in the section describing the examples.

In the following description, all variables are, unless otherwise noted, as defined in the formulas described herein. The following non-limiting descriptions illustrate the general methodologies that may be used to obtain the compounds described herein. The following schemes illustrate synthesis of exemplary compounds of general Formula I(a) and Formula II(a) that incorporate a furanose ring of the β-D-ribose configuration. However, this is not intended to be a limitation on the scope of the present invention. Other configurations are also included in the present invention.

Formula 1 (Formula Ia when R₃=R₄=H and R₇ is H)

General Scheme 1 illustrates the synthesis of compounds of formula Ia. General Scheme 1 starts with the silylation of compounds of formula 1-a with an appropriate reagent (e.g., hexamethyldisilizane or BSA) or alternatively treatment of compounds of formula 1-a with a strong hindered base such as but not limited to DBU. The resulting intermediate is then reacted with compounds of formula 1-b in the presence of a silylated Lewis acid such as trimethylsilyl trifluoromethanesulfonate in an appropriate polar aprotic solvent (e.g., 1,2-dichloroethane or acetonitrile) at a suitable temperature (e.g., elevated temperatures) to produce compounds of formula 1-c. Compounds of formula 1-c undergo hydrogenolysis to produce compounds of formula 1-d using 10% Pd/C and hydrogen gas in the presence of an appropriate base (e.g., triethylamine) in an appropriate solvent such as dioxane at ambient temperatures or by using ammonium formate and 5% Pd/C in an appropriate solvent or a combination of solvents (e.g., methanol and ethyl acetate) at a suitable temperature (e.g., elevated temperatures). Finally, compounds of formula Ia are obtained by deprotecting compounds of formula 1-d by treatment with an appropriate base (e.g., ammonia) in an appropriate polar solvent (e.g., methanol). The appropriate compounds I-a and 1-b may be prepared by methods described in literature with modifications known to one of ordinary skill in the art. For example, compound I-a may be prepared according to methods described in General Scheme 12. One of ordinary skill could prepare compound I-b by methods known in the art.

It will be appreciated by one of ordinary skill in the art that when R₃ and R₄ of the desired final product are not hydrogen, rather they are chemically stable groups defined in Formula Ia, the deprotection step is not required. In addition, when R₇ of the desired final product is not hydrogen, the hydrogenolysis step is not required.

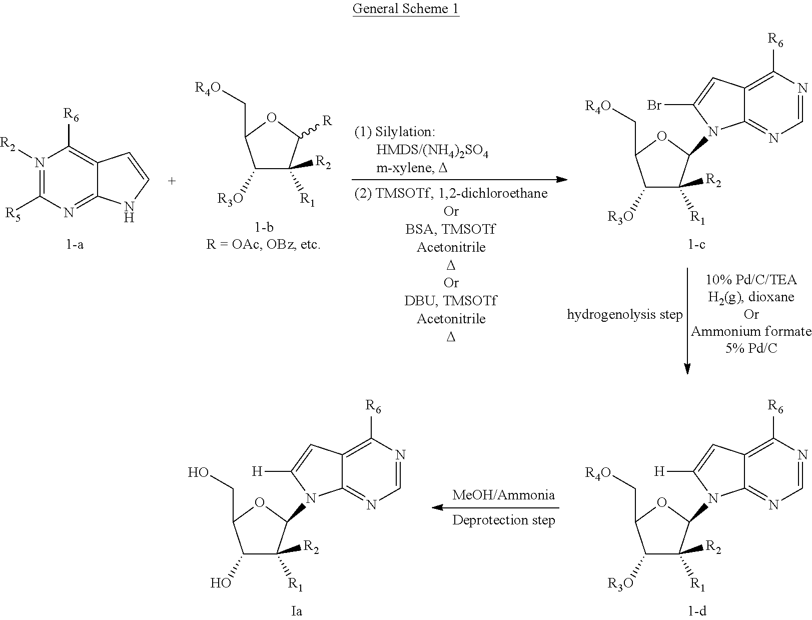

Formula 2 (Formula Ia when R₁ is F)

Compounds of formula 2 may be prepared as described in General Scheme 2. General Scheme 2 starts by treating compounds of formula 2-a with an appropriate deoxyfluorinating agent (e.g., (diethylamino)sulfur trifluoride (DAST) or Deoxofluor) in an appropriate solvent (e.g., dichloromenthane) at a suitable temperature (e.g., ambient temperatures). Compounds of formula 2-a may be prepared according to General Scheme 1 with modifications known to one of ordinary skill in the art.

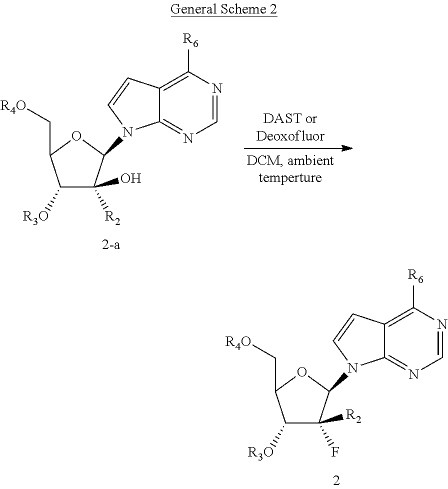

Formula 3 (Formula Ia, when R₆ is H)

Compounds of formula 3 may be prepared according to methods described in General Scheme 3. General Scheme 3 starts by reacting compounds of formula 3-a with sodium nitrite in aqueous acetic acid at elevated temperatures followed by reacting with water at ambient temperature to provide compounds of formula 3-b. Compounds of formula 3-b reacts with an appropriate acylating agent (e.g., acetic anhydride) in an appropriate solvent (e.g., pyridine) to protect any hydroxyl group in compounds of formula 3-b The resulting protected compounds of formula 3-b are reacted with an appropriate chlorinating agent (e.g., phosphoryl trichloride) at a suitable temperatures (e.g., elevated temperatures) to produce compounds of formula 3-c. Compounds of formula 3-c are treated with a suitable base (e.g., ammonia) in an appropriate polar solvent (e.g., methanol) at suitable temperatures (e.g., at or below ambient temperature) to produce compounds of formula 3-d. Finally, compounds of formula 3-d undergo hydrogenolysis using suitable hydrogenating agent (e.g., 10% Pd/C and hydrogen gas in the presence of a base such as sodium bicarbonate) in a suitable solvent (e.g., ethanol) to provide compounds of formula 3.

Compounds of formula 3-a may be prepared according to General Scheme 1 with modifications known to one of ordinary skill in the art.

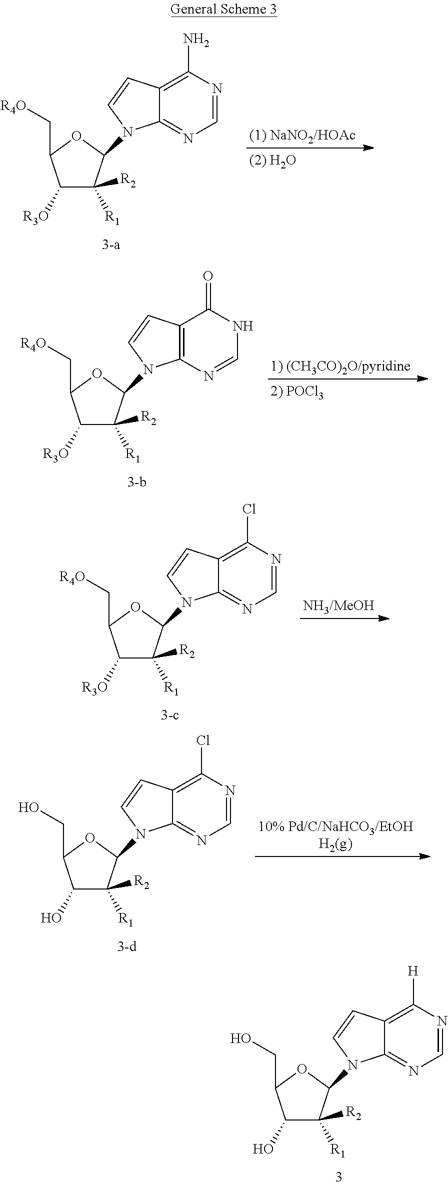

Formulae 4-1 (Formula Ia, R₅ is —C(═O)—NH₂), 4-2 (Formula Ia, R₅ is —C(NH₂)═N—(OH)), 4-3 (Formula Ia, R₅ is —C(═S)—NH₂) and 4-4 (Formula Ia, R₅ is —C(NH₂)═N—(OMe)).

Compounds of formula 4-1 may be prepared as shown in General Scheme 4 using path A. In Scheme 4, path A compounds of formula 4-a are reacted with hydrogen peroxide in aqueous ammonium hydroxide at a suitable temperature (e.g., room temperature). In situations where at least one of OR₃ or OR₄ is an oxygen-protecting group, the reaction mixture from step 1 may need to be deprotected by further reacting with an appropriate base (e.g., ammonia) in a suitable polar solvent (e.g., MeOH) to obtain compounds of formula 4-1.

Compounds of formula 4-2 may be prepared as shown in General Scheme 4 using path B. In Scheme 4, path B compounds of formula 4-a are treated with hydroxylamine (or hydroxylamine hydrochloride and a suitable base such as triethylamine) in a suitable polar solvent (e.g., isopropanol or absolute ethanol) at suitable temperatures (e.g., at elevated temperatures). In situations where at least one of OR₃ or OR₄ is an oxygen-protecting group, the reaction mixture from step 1 may need to be deprotected by further reacting with an appropriate base (e.g., ammonia) in a suitable polar solvent (e.g., MeOH) to obtain compounds of formula 4-2.

Compounds of formula 4-3 may be prepared as shown in General Scheme 4 using path C. In Scheme 4, path C compounds of formula 4-a are treated with hydrogen sulfide gas in the presence of a suitable base (e.g., sodium methoxide) in a suitable solvent (e.g., methanol) at suitable temperature (e.g., ambient temperature) or in the presence of triethylamine in a suitable solvent (e.g., pyridine). Alternatively, compounds of formula 4-a can be treated with sodium hydrogen sulfide in an appropriate solvent such as isopropanol at elevated temperatures. In situations where at least one of OR₃ or OR₄ is an oxygen-protecting group, the reaction mixture from step 1 may need to be deprotected by further reacting with an appropriate base (e.g., ammonia) in a suitable polar solvent (e.g., MeOH) to obtain compounds of formula 4-3.

Compounds of formula 4-4 may be prepared as shown in General Scheme 4 using path D. In Scheme 4, path D compounds of formula 4-a are treated with postassium cyanide in a suitable solvent (e.g., methanol) at a suitable temperature (e.g., at elevated temperatures). In situations where at least one of OR₃ or OR₄ is an oxygen-protecting group, the reaction mixture from step 1 may need to be deprotected by further reacting with an appropriate base (e.g., ammonia) in a suitable polar solvent (e.g., MeOH) to obtain compounds of formula 4-4.

Compounds of formula 4-a may be prepared according to General Scheme 1 with modifications known to one of ordinary skill in the art.

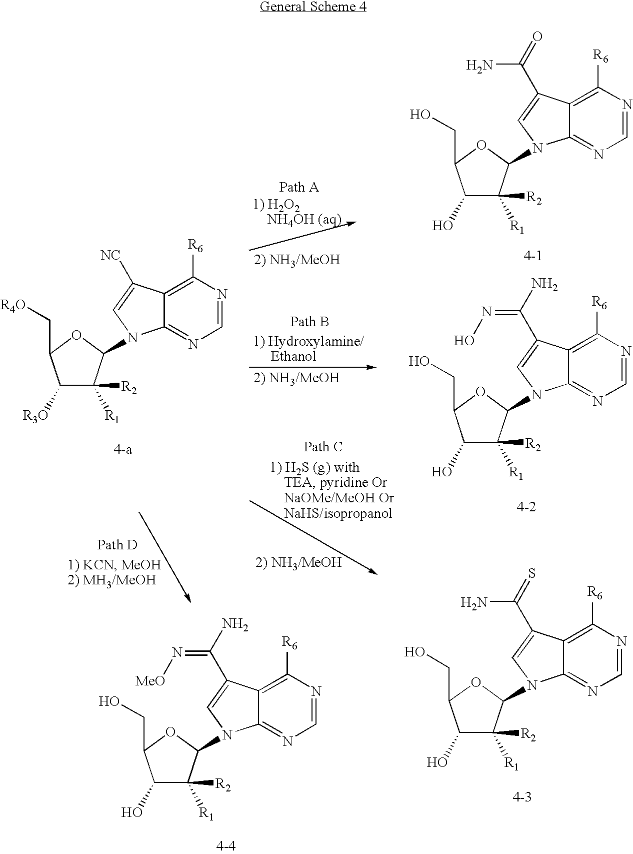

### Formula 5 (Formula Ia)

Compounds of formula 5 may be prepared according to methods described in General Scheme 5. In General Scheme 5 compounds of formula 5-a (Formula Ia) are treated with a suitable oxidant (e.g., m-chloroperoxybenzoic acid) in a suitable solvent (e.g., acetic acid) at a suitable temperature (e.g., an elevated temperature). Compounds of formula 5-a may be prepared according to any of General Schemes 1-4.

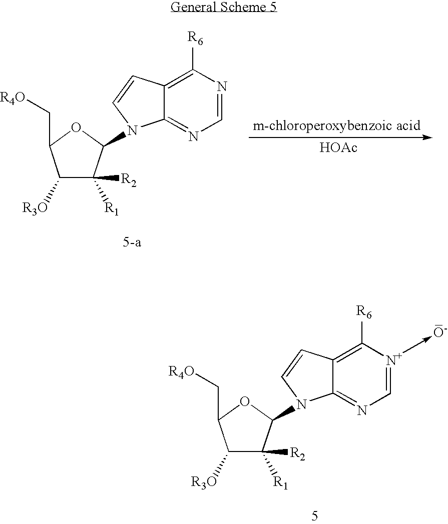

### Formula 6 (Formula Ia, R₃=R₄ are —C(O)ORₐ, —C(O)Rₐ or —C(O)NRₐRₐ′)

Compounds of formula 6 may be prepared according to methods described in General Scheme 6. In General Scheme 6, compounds of formula 6-a are treated with a suitable acylating agent (e.g., methyl chloroformate, ethyl chloroformate, acetic anhydride, propionic anhydride, benzoic anhydride, benzoyl chloride, propionyl chloride) or an appropriately substituted carbamoylimidazolium salts in a suitable solvent (e.g., acetonitrile or THF) in the presence of a suitable base (e.g., triethylamine or pyridine) optionally with 4-dimethylaminopyridine (DMAP) at a temperature from 0° C. to the reflux temperature of the solvent. Compounds of formula 6-a may be prepared according to General Scheme 1.

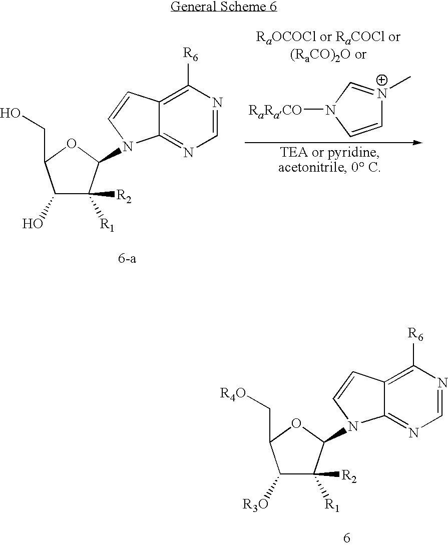

### Formula 7 (Formula Ia, OR₃ is OCOCH(R_{q})NRₕRₒ and OR₄ is OH)

Compounds of formula 7 may be prepared according to methods described in General Scheme 7. In General Scheme 7 compounds of formula 7-a are treated with a suitable protecting agent (e.g., silylating agent such as tert-butyl diphenylchlorosilane (TBDPSCl)) in the presence of imidazole in a suitable solvent (e.g., pyridine) at a suitable temperature (e.g., ambient temperature) to provide compounds of formula 7-b. Compounds of formula 7-b can be reacted with a suitable substituted amino acid in the presence of 4-dimethylaminopyridine (DMAP) and a suitable activating agent (e.g., 1-ethyl-3-(3-dimethylaminopropyl)carbodiimide (EDC)) in a suitable solvent (e.g., acetonitrile or dimethylformamide (DMF)) to produce compounds of formula 7-c. Compounds of formula 7-c are selectively deprotected to remove the protecting group of the hydroxyl moiety (e.g., tert-butyl diphenylsilyl group) by using a suitable reagent (e.g., ammonium fluoride) in a suitable solvent (e.g., methanol) at a suitable temperature (e.g., at elevated temperatures) to provide compounds of formula 7-d. Compounds of formula 7-d can be subsequently reacted with an appropriate acid to provide a salt, compounds of formula 7-1. Compounds of formula 7-a may be prepared as described in General Schemes 1 and 3.

For compounds of formula 7-d wherein at least one of Rₕ or Rₒ is a protecting group as defined in Formula I (a) (e.g., a tert-butyloxycarbonyl (BOC) or carbobenzyloxy (Cbz) group), then the protecting group may be optionally removed to provide a free amine which may subsequently react with an appropriate acid to provide a salt, compounds of formula 7-2.

For compounds of formula 7-d, when both Rₕ and Rₒ are protecting groups, the protecting group may be selectively removed to provide a free amine (—NHRₒ or —NHRₕ), which may subsequently react with an appropriate acid to provide a salt, compounds of formula 7-3. Alternatively, both protecting groups may be removed to provide a free amine (—NH₂) which may subsequently react with an appropriate acid to provide a salt, compounds of formula 7-4. The choice of protecting groups and the reaction conditions of deprotecting step is known to one skilled in the art in view of the structure of the compounds.

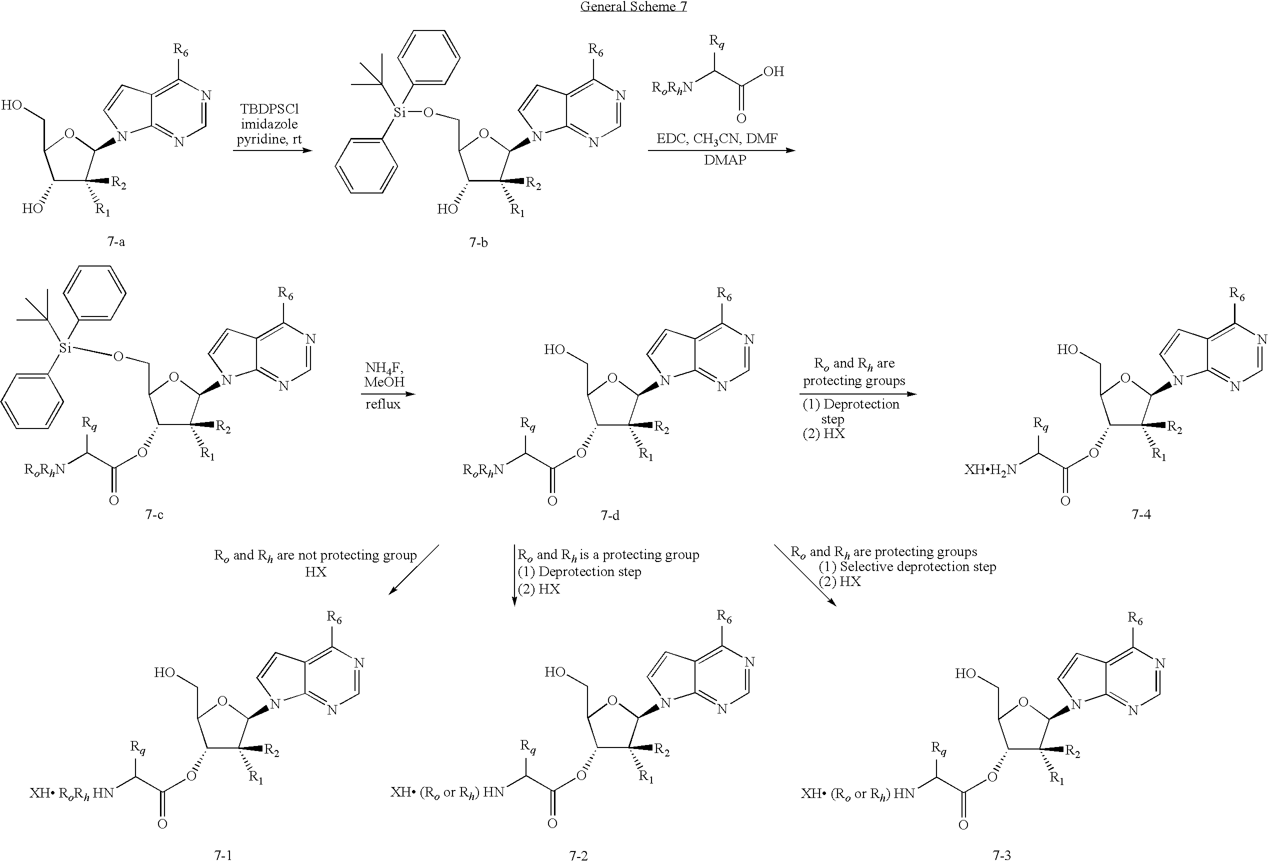

Formula 8 (Formula I(a), where R₃=H and R₄=OP(O)(ORₑ)N(R_{d})CH(R_{b})COOR_{c})

Compounds of formula 8 may be prepared according to methods described in General Scheme 8. In General Scheme 8, compounds of formula 8-a are treated with an appropriate protecting agent (e.g., silylating agent such as TBDMSCl) in the presence of a suitable base (e.g., imidazole) in a suitable solvent (e.g., DMF) to provide compounds of formula 8-b. Compounds of formula 8-a may be prepared as described in any of General Schemes 1 and 3. Compounds of formula 8-b can undergo reactions with levulinic acid in the presence of DMAP and a suitable activating agent (e.g., N,N′-dicyclohexylcarbodiimide (DCC)) in a suitable solvent (e.g., ethyl acetate) to produce compounds of formula 8-c. Compounds of formula 8-c can be treated with a suitable activating agent to selectively remove the protecting group (e.g., a mixture of tetrabutylammonium fluoride (TBAF) and acetic acid in THF may be used to remove the silyl protecting group) to produce compounds of formula 8-d. Compounds of formula 8-d react with an appropriately substituted phosphoramido chloridate in the presence of a suitable base (e.g., N-methylimidazole) in a suitable solvent (e.g., THF) at a suitable temperature (ambient temperature) to produce compounds of formula 8-e. Finally, compounds of formula 8-e can be treated with a suitable agent (e.g., 2M hydrazine hydrate in a pyridine-acetic acid buffer) by selective removal of the levulinate group to provide compounds of formula 8.

Alternatively, compounds of formula 8-a can be reacted with an appropriately substituted phosphoramido chloridate in the presence of a suitable base (e.g., N-methylimidazole) in a suitable solvent such as dioxane at a suitable temperature (ambient temperature) to give compounds of formula 8.

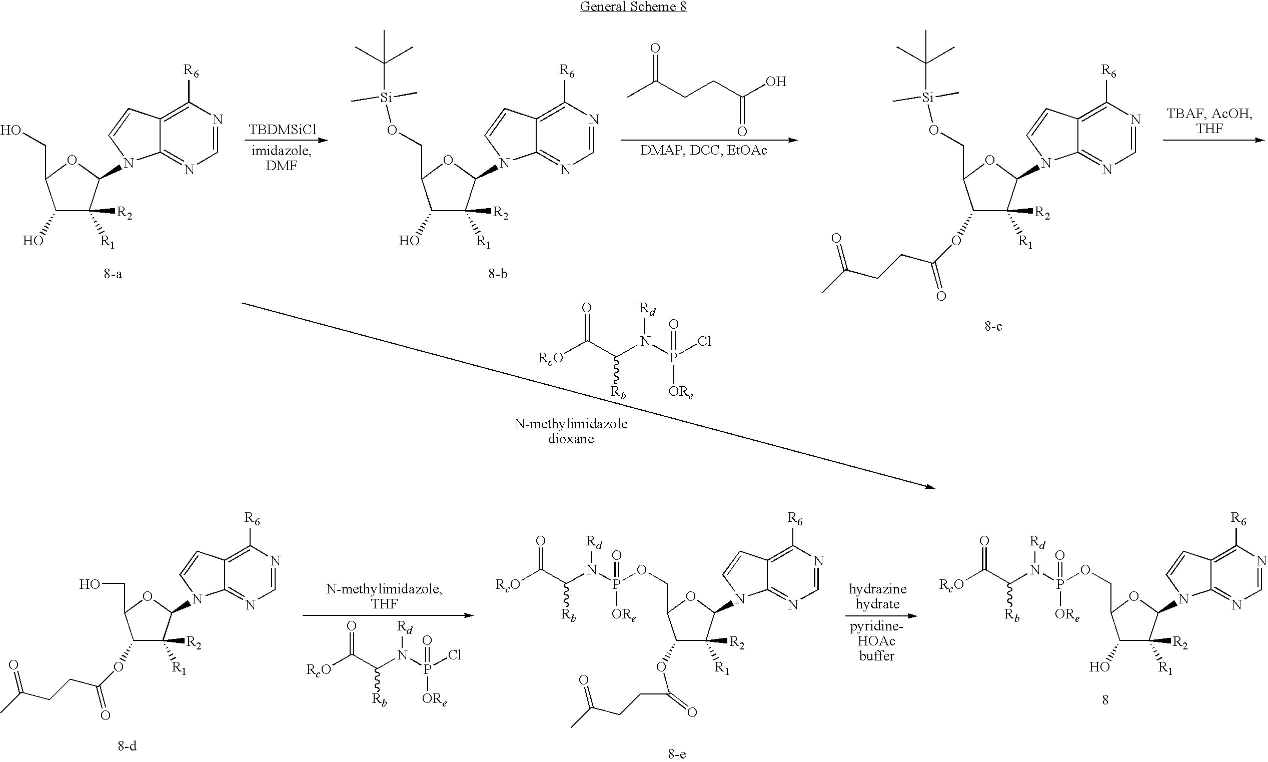

Formula 9 (Formula I(a), Where OR₃═OH and OR₄═OC(UY)P(O)(OH)N(R_{d})CH(R_{b})COOR_{c} Where R_{b}, R_{c} and R_{d} are as Described in Formula I (a), U and Y can be Independently Hydrogen or Fluorine)

Compounds of formula 9 may be prepared according to methods described in General Scheme 9. In General Scheme 9, compounds of formula 9-a are treated with (OEt)₂(O)P(CUY)OT_{f} (where U and Y can be independently hydrogen or fluorine and R₈ is a protected hydroxyl group as defined in Formula Ia) in the presence of a suitable base (e.g., sodium hydride) in a suitable solvent (e.g., THF) at about −78° C. to produce compounds of formula 9-b. Compounds of formula 9-b react with trimethylsilyl bromide (TMSBr) in acetonitrile at ambient temperature followed by treatment with an exchange resin such as Dowex-H⁺ in a suitable solvent (e.g., methanol) at a suitable temperature (elevated temperatures) to produce compounds of formula 9-c. Compounds of formula 9-c can be treated with a substituted amino acid ester in the presence of an activating agent (e.g., DCC) optionally with DMAP in a suitable solvent (e.g., tert-butyl alcohol) at a suitable temperature (elevated temperatures) followed by removal of the protecting group of R₈ with the appropriate reagents to provide compounds of formula 9. Compounds of formula 9-a may be prepared according to General Scheme 1 with modifications known to one of ordinary skill in the art.

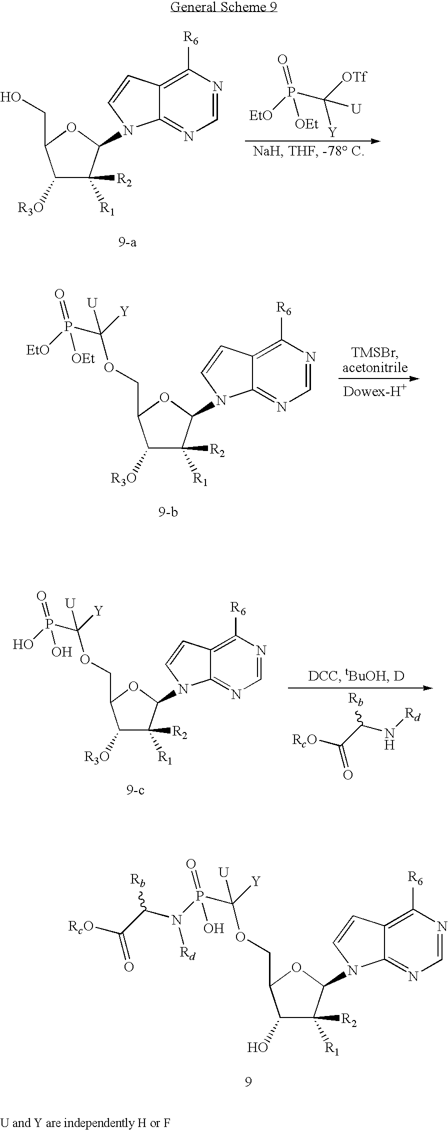

### Formula 10 (Formula Ia, Where R₇=NRₐRₐ)

Compounds of formula 10 may be prepared according to methods described in General Scheme 10. General Scheme 10 begins with reaction of compounds of formula 10a with liquid ammonia in a sealed vessel at a suitable temperature (elevated temperatures) to provide compounds of formula 10-b. Compounds of formula 10 may be obtained by reductive amination of compounds of formula 10-b with the appropriately substituted aldehyde or ketone using a suitable reducing agent (e.g., sodium triacetoxyborohydride in the presence of acetic acid) in a suitable solvent (e.g., 1,2-dichloroethane, dichloromethane or acetonitrile). Compounds of formula 10-a may be prepared according to General Scheme 1 with modifications known to one of ordinary skill in the art.

It should be appreciated by one of ordinary skill in the art that compounds of formula 10-a, wherein R₇ is Br may be reacted directly with amines (e.g., NHRₐRₐ) in the presence of a suitable base (e.g., triethylamine) in a suitable solvent such as acetonitrile or 1,2-dichloroethane) at a suitable temperature (e.g., ambient temperature to elevated temperatures) to give compounds of formula 10. Furthermore, compounds of formula 10-a, wherein R₇ is Br can be used as an intermediate to react with a variety of reagents to provide compounds of Formula Ia where R₇ can be a variety of different substituents defined in Formula Ia, for example, General Scheme 11 described below.

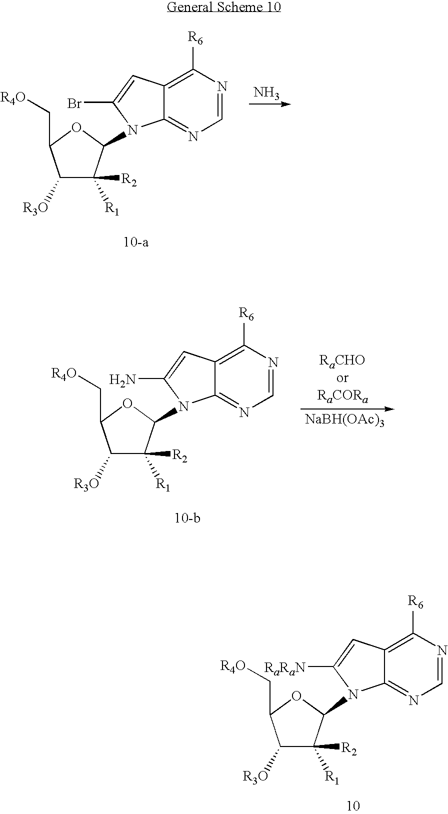

### Formula 11 (Formula Ia Where R₇ is as Described Herein)

Some compounds of Formula Ia (Compound II in General Scheme 11) may be prepared according to methods described in General Scheme 11. General Scheme 11 begins by reaction of compounds of formula 10-a with bis(pinacolato)diborane (B₂pin₂) in the presence of a suitable catalyst (e.g., [Ir(COD)OMe]₂) and 4,4′-di-tert-butylbipyridine (dtbpy) in a suitable solvent (e.g., THF) at a suitable temperature (elevated temperatures) to produce compounds of formula 11-b. Then compounds of formula 11-b reacts through a Suzuki reaction by reacting with an appropriately substituted reactant (e.g., ArX where X is a suitable leaving group such as Br, I, OTf, etc.) in the presence of a suitable catalyst (e.g., Pd(dppf)Cl₂) and a base (e.g., potassium carbonate) in a suitable solvent (e.g., DMF) at a suitable temperature (elevated temperatures) to produce compounds of formula 11. It should be understood that suitable boron reagents other than Bpin may also be employed in these reactions. Compounds of formula 10-a may be prepared as described in General Scheme 1.

It should be appreciated by one of ordinary skill in the art that an intermediate such as compounds of formula 11-b may be reacted with a variety of reagents (e.g., haloacetylenes, vinyl halides) to give compounds of Formula Ia where R₇ is a variety of substituents. Furthermore, compounds of formula 10-a may also react with a variety of reagents under, for example, but are not limited to Heck or Sonogashira reaction conditions to prepare compounds of Formula Ia where R₇ is as described herein.

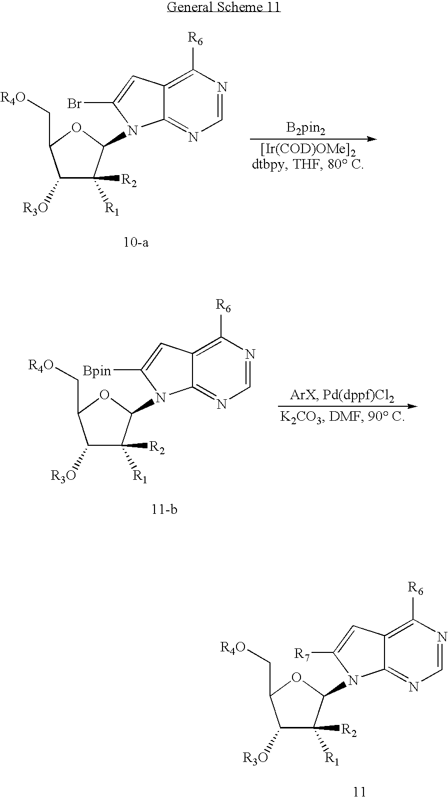

### Formula 12 (Formula Ia)

Compounds of formula 12 may be prepared according to General Scheme 12. General Scheme 12 begins with halogenation of compounds of formula 12-a using a suitable halogenating agent such as bromine or iodine in a suitable solvent (e.g., DMF) to generate compounds of formula 12-b. Compounds of formula 12-a may be prepared by methods known to one skilled in the art. For example, when R₅ is H, compounds of formula 12-a may be prepared according to methods described in International Publication No. WO2008/044130 to Salituro et al. Then, compounds of formula 12-b are treated with sodium hydride in a suitable solvent (e.g., THF) followed by reaction with p-toluenesulfonyl chloride to produce compounds of formula 12-c. Compounds of formula 12-c react with a suitable boron agent (e.g., bis(pinacolato)diborane (B₂pin₂)) in the presence of a catalyst (e.g., Pd(dppf)Cl₂) in the presence of a suitable base (e.g., KOAc) in a suitable solvent (e.g., DME) at a suitable temperature (elevated temperatures or a microwave reactor can be employed to reach the suitable temperature) to produce compounds of formula 12-d. Finally, compounds of formula 12-d undergo Suzuki reaction with the appropriately substituted reactant (e.g., ArX where X is a leaving group such as Br, I, or OTf) and a suitable catalyst (e.g., Pd(dppf)Cl₂) in the presence of a base (e.g., K₂CO₃) in an appropriate solvent (e.g., DMF) at a suitable temperature (elevated temperatures) to produce compounds of formula 12. Compounds of formula 12 may further go through a coupling reaction as described in General Scheme 1.

It should be appreciated by one of ordinary skill in the art that an intermediates such as 12-d may react with a variety of reagents such as, but are not limited to, haloacetylenes, vinyl halides, etc. to provide compounds of formula 12. Furthermore, compounds of formula 12-c may react with a variety of reagents under, for example, but are not limited to Heck or Sonogashira reaction conditions to provide compounds of formula 12.

It should also be understood that compounds of Formula Ia, where R₅ is Br or I may be prepared by reacting compounds of formula 12-b with a compounds of formula 1-b as described in General Scheme 1. Compounds of Formula Ia where R₅ is Br or I may undergo a variety of reactions as described in General Schemes 9-11 to give compounds of Formula Ia wherein R₅ is a variety of substituents. Furthermore, compounds of formula Ia where R₅ contains a reactive group such as but not limited to an acid moiety can be manipulated by one skilled in the art of organic synthesis to give compounds of Formula Ia where R₅ is for example but is not limited to a thiophene carboxamide.

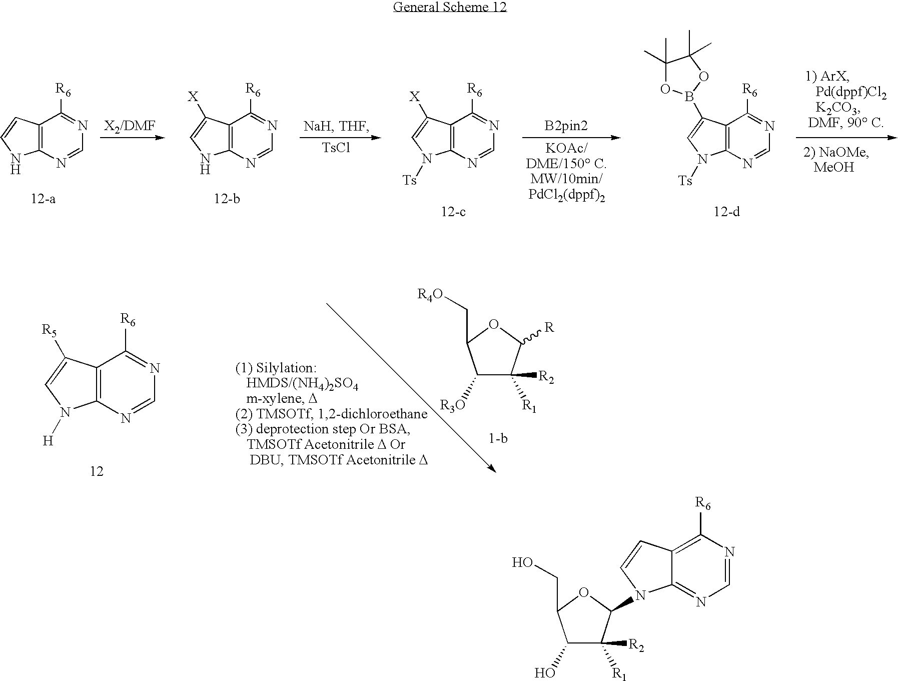

### Alternative Synthesis of Formula Ia

It will be appreciated by one of ordinary skill in the art that a compound of Formula Iab where R₆ is Br or I can undergo a variety of reactions as described in General Schemes 9-11 to give compounds of Formula Iab wherein R₆ can be a variety of substituents (as shown in General Scheme 13).

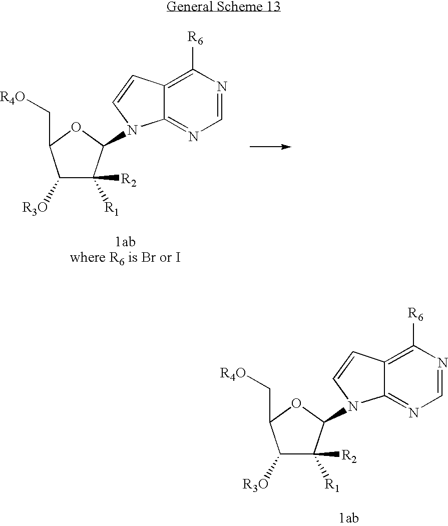

Formula Ia Where OR₃═OH and OR₄═OP(═O)(OH)O(CH₂)ₛ—O—(CH₂)ᵥCH₃ Where s is 2-6 and v is 11-25, or OR₃ and OR₄ Can Form a Cyclic Phosphate Where s and v are as Described Herein

Compounds of formula 14 may be prepared according to methods described in General Scheme 14. Compounds of formula Ia can be reacted with a suitable reagent (e.g., Cl₂P(═O)O(CH₂)ₛ—O—(CH₂)ᵥCH₃ where s is 2 to 6 and v is 11 to 25) in the presence of a suitable base (e.g., LiHMDS) in a suitable solvent (e.g., THF) at a suitable temperature (ambient temperature) to provide compounds of formula 14. Compounds of formula Ia may be prepared as shown in General Scheme 1 through 7 and 10 through 13.

It should be appreciated by one skilled in the art of organic synthesis that compounds of formula 14 where O(CH₂)ₛ—O—(CH₂)ᵥCH₃ and compounds of formula 14-1 can be treated with a suitable reagent such as but not limited to potassium tert-butoxide in a suitable solvent such as DMSO at a suitable temperature such as ambient temperature to give compounds of formula 14-2. Compounds of formula 14-2 can be treated with an appropriate reagent such as X(CH₂)ₛ—O—(CH₂)ᵥCH₃ where X is a leaving group such as Br, Cl, I, or OTf in a suitable solvent such as dimethylformamide in the presence of a base such as but not limited to N,N-diisopropylethylamine at a suitable temperature such as 60° C. to give compounds of formula 14-3.

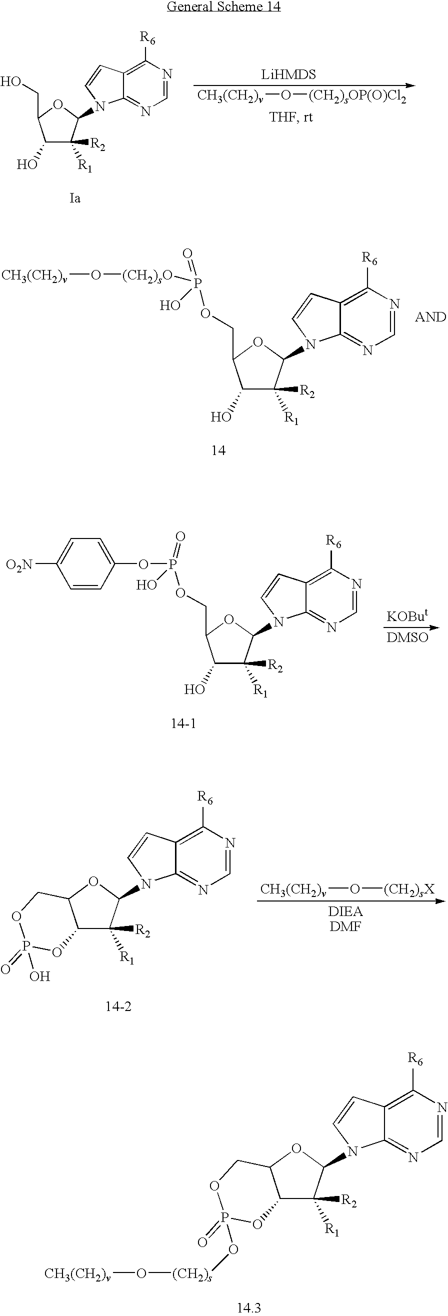

### Formula Ia Where R₂ is N₃

Compounds of Formula Ia where R₂ is N₃ may be prepared according to methods described in General Scheme 15. General Scheme 15 begins with reaction of compounds of formula Ia where R₃=R₄=H with a suitable protecting group such as TIPDSi-Cl₂ in the presence of a suitable base such as pyridine in a suitable solvent such as pyridine at a suitable temperature such as 0° C. to ambient temperature to give compounds of formula 15-1. Compounds of formula 15-1 can be activated by treatment with a suitable acylating agent such as triflic anhydride in the presence of a suitable base such as pyridine in a suitable solvent such as pyrindine at a suitable temperature such as −10° C. to ambient temperature to give compounds of formula 15-2. Compounds of formula 15-2 react with a suitable reagent such as sodium azide in a suitable solvent such as dimethylformamide at a suitable temperature such 60-80° C. to produce compounds of formula 15-3. Compounds of formula 15-3 can be deprotected by reaction with a suitable desilylating agent such as tetrabutylammonium fluoride in a suitable solvent such as tetrahydrofuran at a suitable temperature such as 0° C. to ambient temperature to give compounds of formula Iac where R₂ is N₃.

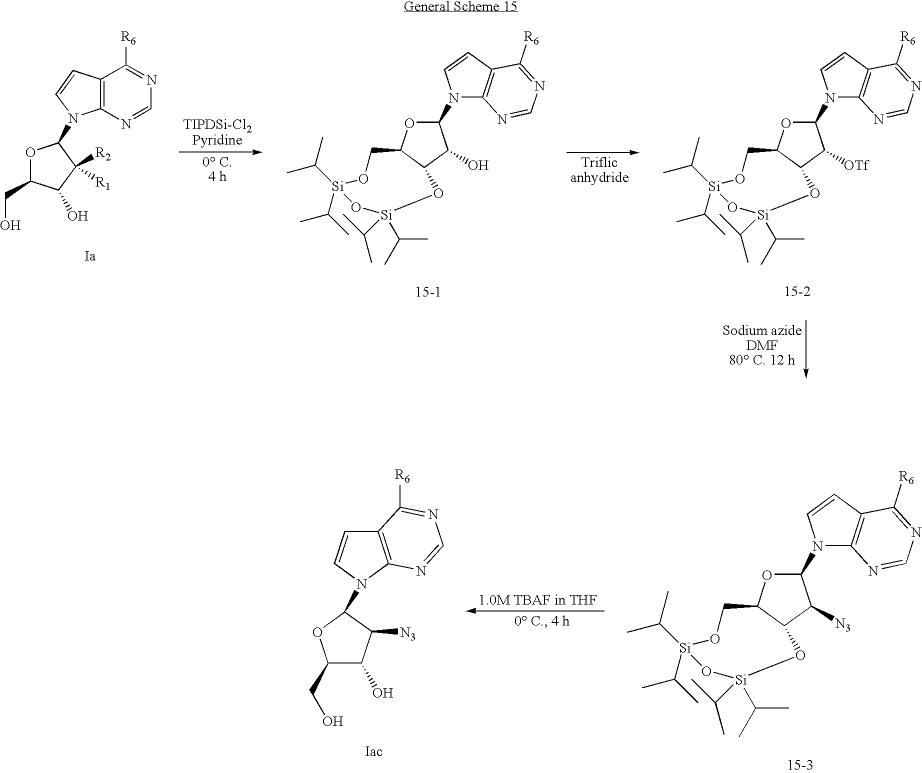

### Formula Ia Where R₁ and OR₃ Form a Cyclic Carbonate

Compounds of formula 16 where R₁ and OR₃ form a cyclic carbonate can be prepared according to the methods described in General Scheme 16. General Scheme 16 begins with the selective protection of compounds of formula Iad where R₁=R₃=R₄=H by reaction with a suitable protecting agent such as TBDMS-Cl in a suitable solvent such as dimethylformamide at a suitable temperature such as 0° C. to ambient temperature to give compounds of formula 16-1. Compounds of formula 16-1 can be treated with a suitable reagent such as carbonyl diimidazole in a suitable solvent such as dimethylformamide to produce compounds of formula 16-2. Compounds of formula 16-2 can be deprotected with a suitable reagent such as tetrabutylammonium fluoride in a suitable solvent such as tetrahydrofuran at a suitable temperature such as 0° C. to ambient temperature or with boron trichloride in a suitable solvent such as tetrahydrofuran/2-chloroethanol mixtures at a suitable temperature such as 0° C. to ambient temperature to give compounds of formula Ia where R₁ and OR₃ form a cyclic carbonate.

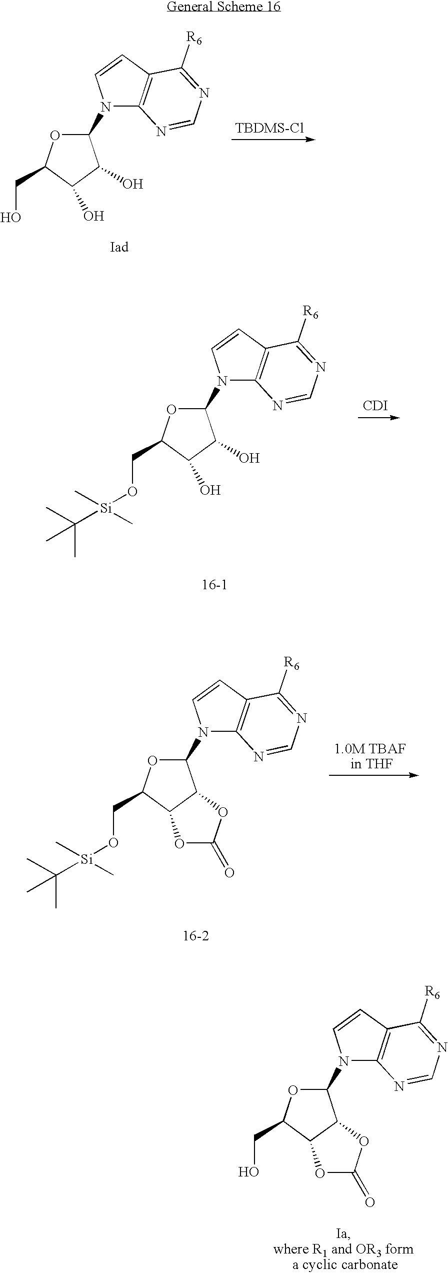

The present invention will now be described in more detail with reference to the following examples. However, these examples are given for the purpose of illustration and are not to be construed as limiting the scope of the invention.

### EXAMPLES

### Example 1

### Preparation of (2R,3R)-2-(4-amino-5-cyano-7H-pyrrolo[2,3-d]pyrimidin-7-yl)-5-(benzoyloxymethyl)-3-methyltetrahydrofuran-3,4-diyl dibenzoate, 1

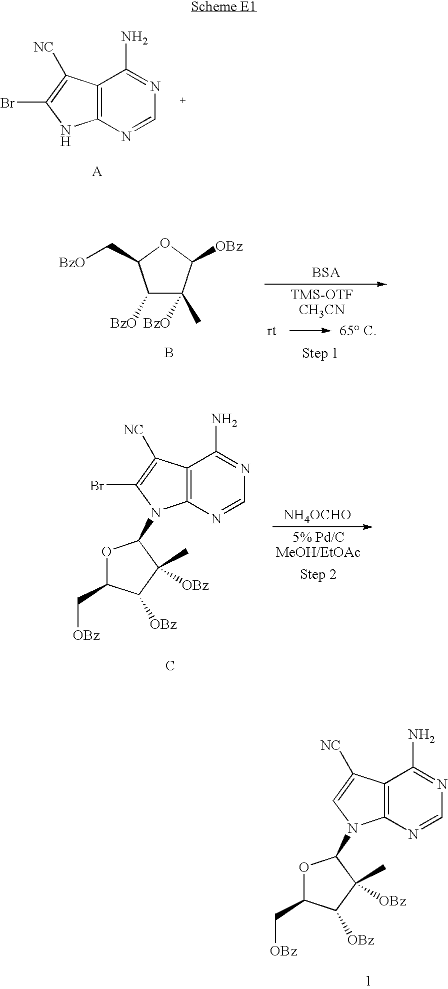

### Step 1: Preparation of (2R,3R)-2-(4-amino-6-bromo-5-cyano-7H-pyrrolo[2,3-d]pyrimidin-7-yl)-5-(benzoyloxymethyl)-3-methyltetrahydrofuran-3,4-diyl dibenzoate, C

To a suspension of 4-amino-6-bromo-7H-pyrrolo[2,3-d]pyrimidine-5-carbonitrile, A (4.1 g, 0.017 mol) in acetonitrile (120 mL) at room temperature was added via syringe BSA (6.9 g, 0.034 mol) over a 20 min. period. The mixture was stirred at room temperature for 30 min. after which (2S,3R,4R,5R)-5-(benzoyloxymethyl)-3-methyltetrahydrofuran-2,3,4-triyl tribenzoate, B (10.0 g, 0.17 mol) was added in one portion followed by addition via syringe of TMS-OTf (11.3 g, 0.051 mol) over a 15 min. period. The mixture was stirred at room temperature for 15 min. and then heated to 65° C. for 17 hr. The reaction mixture was diluted with ethyl acetate (120 mL) and the mixture was poured into saturated aqueous sodium bicarbonate solution (120 mL). After stirring for 20 min., the phases were separated and the aqueous phase was extracted with ethyl acetate. The combined organic phase was washed with brine and dried over sodium sulfate. The mixture was filtered and the filtrate was evaporated in vacuo to give 15.8 g of crude product as a brown foam. The residue was dissolved in ethyl acetate, silica gel was added and the mixture was concentrated in vacuo. The residue was transferred to a pre-column and purified by silica gel chromatography using a stepwise gradient from hexanes to 40% ethyl acetate/hexanes to give 7.72 g (65%) of (2R,3R)-2-(4-amino-6-bromo-5-cyano-7H-pyrrolo[2,3-d]pyrimidin-7-yl)-5-(benzoyloxymethyl)-3-methyltetrahydrofuran-3,4-diyl dibenzoate, C as a yellow foam. ¹H NMR indicated this was a mixture of anomers.

### Step 2: Preparation of (2R,3R)-2-(4-amino-5-cyano-7H-pyrrolo[2,3-d]pyrimidin-7-yl)-5-(benzoyloxy methyl)-3-methyltetrahydrofuran-3,4-diyl dibenzoate, 1

To a mixture of 5% Pd/C (0.1 g) in a small amount of ethyl acetate under a stream of nitrogen was added (2R,3R)-2-(4-amino-6-bromo-5-cyano-7H-pyrrolo[2,3-d]pyrimidin-7-yl)-5-(benzoyloxymethyl)-3-methyltetrahydrofuran-3,4-diyl dibenzoate, C (7.72 g, 0.011 mol) in ethyl acetate (50 mL). To this mixture was added ammonium formate (6.93 g, 0.11 mol) in one portion. Methanol (50 mL) was added in a steady stream over a 1 min. period and the mixture was stirred a room temperature for 30 min., then heated to reflux for 23 hr. The mixture was cooled to 35° C. and filtered through a pad of celite. The filtrate was washed with water, brine and dried over sodium sulfate. The mixture was concentrated to give 7.15 g of crude material. Initial attempts to purify this material by silica gel chromatography using ethyl acetate/toluene (3:7) failed. The recovered material was purified by silica gel chromatography eluting with gradient from dichloromethane to ethyl acetate/dichloromethane (2:8). The fractions containing the major component (slower running material) were combined and concentrated in vacuo to give 2.33 g (34%) of (2R,3R)-2-(4-amino-5-cyano-7H-pyrrolo[2,3-d]pyrimidin-7-yl)-5-(benzoyloxy methyl)-3-methyltetrahydrofuran-3,4-diyl dibenzoate, 1 as a white solid/foam. NMR analysis confirms this to be the β-anomer. ¹H NMR (DMSO-d₆) δ 8.52 (S, 1H), 8.36 (s, 1H), 7.99-8.05 (m, 4H), 7.83-7.86 (m, 2H), 7.47-7.67 (m, 7H), 7.37 (br t, 2H), 6.99 (br s, 2H), 6.89 (s, 1H), 5.94 (s, 1H), 4.79 (m, 3H), 1.54 (s, 3H).

### Example 2

### Preparation of (2R,3R)-2-(4-amino-5-cyano-7H-pyrrolo[2,3-d]pyrimidin-7-yl)-5-(isobutyryloxymethyl)-3-methyltetrahydrofuran-3,4-diyl bis(2-methylpropanoate), 2

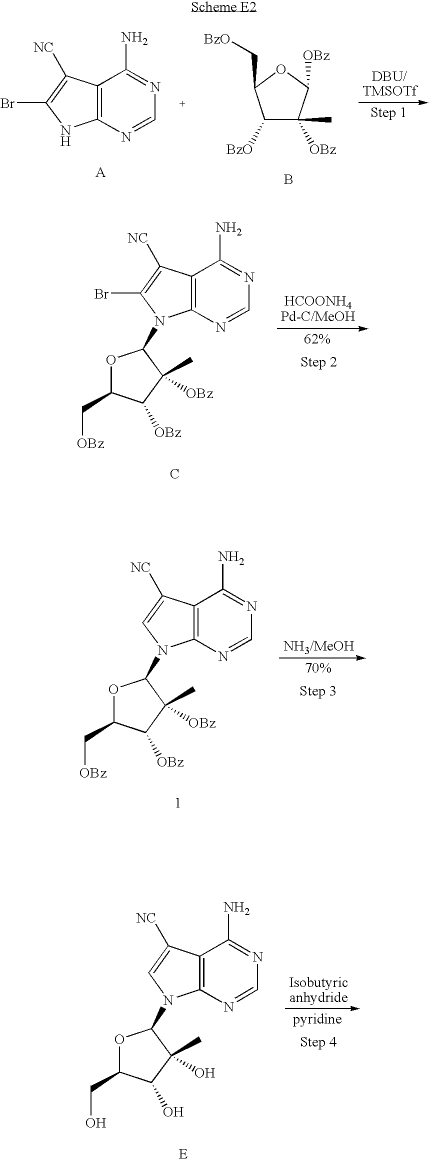

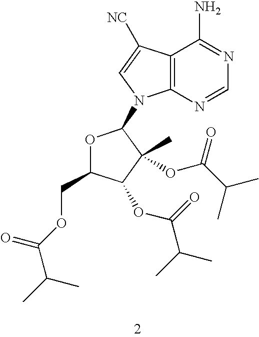

### Step 1: Preparation of (2R,3R)-2-(4-amino-6-bromo-5-cyano-7H-pyrrolo[2,3-d]pyrimidin-7-yl)-5-(benzoyloxymethyl)-3-methyltetrahydrofuran-3,4-diyl dibenzoate, C

To a suspension of 4-amino-6-bromo-7H-pyrrolo[2,3-d]pyrimidine-5-carbonitrile, A (5.3 g, 22.0 mmol), (2R,3R,4R)-5-(benzoyloxymethyl)-3-methyltetrahydrofuran-2,3,4-triyl tri benzoate, B (12.8 g, 22.0 mmol) in anhydrous acetonitrile (200 ml) was added DBU (10 ml, 66.0 mmol). The mixture was cooled to 0° C. and TMSOTf (15.9 ml, 88.0 mmol) was added dropwise. The mixture was stirred at room temp for 15 min and then heated at 65° C. for 2 h. The mixture was cooled to room temperature, a saturated aqueous NaHCO₃ solution (200 ml) was added and the reaction mixture was extracted with EtOAc (2×150 ml). The organics were dried over Na₂SO₄ and concentrated to give orange residue. The orange residue was dissolved in minimal amount of CH₂Cl₂ and loaded onto a column packed with silica/CH₂Cl₂ and eluted with CH₂Cl₂/EtOAc (9:1→3:1). Two products with required product mass were obtained: A) 9.7 g, 63% [non-polar on TLC eluting with 4:1 CH₂Cl₂/EtOAc] N1-regio product; 2.9 g, 19% [polar on TLC eluting with 4:1 CH₂Cl₂/EtOAc] of (2R,3R)-2-(4-amino-6-bromo-5-cyano-7H-pyrrolo[2,3-d]pyrimidin-7-yl)-5-(benzoyloxymethyl)-3-methyltetrahydrofuran-3,4-diyl dibenzoate, C.

### Step 2: Preparation of (2R,3R)-2-(4-amino-5-cyano-7H-pyrrolo[2,3-d]pyrimidin-7-yl)-5-(benzoyloxymethyl)-3-methyltetrahydrofuran-3,4-diyl dibenzoate, 1

A mixture of (2R,3R)-2-(4-amino-6-bromo-5-cyano-7H-pyrrolo[2,3-d]pyrimidin-7-yl)-5-(benzoyloxy methyl)-3-methyltetrahydrofuran-3,4-diyl dibenzoate, C (7.4 g, 10.6 mmol), ammonium formate (6.7 g, 106 mmol), and 10 wt % Pd on carbon (700 mg) in methanol (150 ml) and EtOAc (150 ml) was heated at 65° C. for 20 h. The mixture was cooled to ambient temperature and filtered through Celite. The filtered material was washed with methanol (100 ml). The combined filtrate was concentrated to afford an orange solid. Chromatographic purification by silica gel column eluting with 10% EtOAc/CH₂Cl₂ afforded 4.1 g (62%) of (2R,3R)-2-(4-amino-5-cyano-7H-pyrrolo[2,3-d]pyrimidin-7-yl)-5-(benzoyloxymethyl)-3-methyltetrahydrofuran-3,4-diyl dibenzoate, 1 as a yellow foam-solid.

### Step 3: Preparation of 4-amino-7-((2R,3R)-3,4-dihydroxy-5-(hydroxymethyl)-3-methyl tetra hydro furan-2-yl)-7H-pyrrolo[2,3-d]pyrimidine-5-carbonitrile, E

A mixture of (2R,3R)-2-(4-amino-5-cyano-7H-pyrrolo[2,3-d]pyrimidin-7-yl)-5-(benzoyl oxy methyl)-3-methyltetrahydrofuran-3,4-diyl dibenzoate, 1 (1.0 g, 1.6 mmol), and 2M NH₃ in methanol (20 ml) was stirred at room temp for 16 h. The mixture was concentrated to dryness and the residue was suspended in EtOAc/hexanes (2:1) and filtered. The filtered solid was washed with EtOAc/hexanes (2:1) and dried under vacuum to give 346 mg (70%) of 4-amino-7-((2R,3R)-3,4-dihydroxy-5-(hydroxymethyl)-3-methyltetrahydrofuran-2-yl)-7H-pyrrolo[2,3-d]pyrimidine-5-carbonitrile, E as a white solid. ¹H NMR and LC-MS revealed this product is mixture of 4-amino-7-((2R,3R)-3,4-dihydroxy-5-(hydroxymethyl)-3-methyltetrahydrofuran-2-yl)-7H-pyrrolo[2,3-d]pyrimidine-5-carbonitrile, E and its corresponding methanol adduct (˜1:1).

### Step 4: Preparation of (2R,3R)-2-(4-amino-5-cyano-7H-pyrrolo[2,3-d]pyrimidin-7-yl)-5-(isobutyryloxymethyl)-3-methyltetrahydrofuran-3,4-diyl bis(2-methylpropanoate), 2

4-amino-7-((2R,3R)-3,4-dihydroxy-5-(hydroxymethyl)-3-methyltetrahydrofuran-2-yl)-7H-pyrrolo[2,3-d]pyrimidine-5-carbonitrile, E with its methanol adduct (153 mg, 0.5 mmol) in pyridine (5 mL) was added isobutyric anhydride (0.29 ml, 1.6 mmol, 3.2 equiv.). The reaction was stirred at room temperature for 16 h. LC-MS analysis indicated a mixture of 4-amino-7-((2R,3R)-3,4-dihydroxy-5-(hydroxymethyl)-3-methyltetrahydrofuran-2-yl)-7H-pyrrolo[2,3-d]pyrimidine-5-carbonitrile, E and the diacylated product of 4-amino-7-((2R,3R)-3,4-dihydroxy-5-(hydroxymethyl)-3-methyltetrahydrofuran-2-yl)-7H-pyrrolo[2,3-d]pyrimidine-5-carbonitrile, E (with methanol adduct), and no product 2. Another 1 equiv of isobutyric anhydride was added and the mixture was stirred at room temperature for another 24 h. Another 2 equivalent of isobutyric anhydride was added and the mixture was stirred at room temperature for another 24 h. LC-MS analysis indicated a mixture of diacylated product of E (with methanol adduct), and product 2 (with methanol adduct). The mixture was quenched with saturated aqueous NaHCO₃ and extracted with CH₂Cl₂ (2×20 ml). The Organic phase was dried over Na₂SO₄ and concentrated to afford an orange oil. The orange oil was dissolved in CH₂Cl₂ (2 ml) and loaded onto a column packed with silica/CH₂Cl₂. The column was eluted with 4:1→1:1 of CH₂Cl₂/EtOAc to give 8 mg, of (2R,3R)-2-(4-amino-5-cyano-7H-pyrrolo[2,3-d]pyrimidin-7-yl)-5-(isobutyryloxymethyl)-3-methyl tetrahydrofuran-3,4-diylbis(2-methyl propanoate), 2; H¹ NMR (300 MHz, DMSO-d6): 8.40 (s, 1H), 7.8 (s, 1H), 6.60 (s, 1H), 5.69 (bs, 2H), 5.56 (d, 1H), 4.51 (m, 1H), 4.43 (m, 1H), 4.33 (m, 1H), 2.63 (m, 3H), 1.33 (s, 3H), 1.20 (m, 18H). MS: 516.3 (M+1). This material contains a minor amount of its methanol adduct. Two other components were isolated. First component, 10 mg of the tetraacylated product with acylation at amino group and the second component B, 76 mg of the diacyl product along with the methanol adduct of the triacyl product.

### Example 3

### Preparation of (4R,5R)-2-(acetoxymethyl)-5-(4-chloro-5-cyano-7H-pyrrolo[2,3-d]pyrimidin-7-yl)tetrahydrofuran-3,4-diyl diacetate, 3

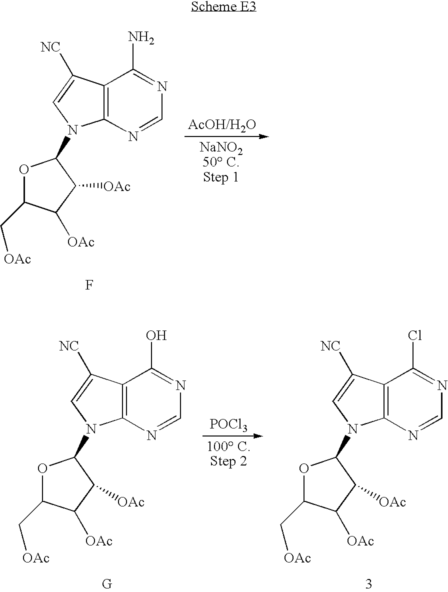

### Step 1: Preparation of (4R,5R)-2-(acetoxymethyl)-5-(5-cyano-4-hydroxy-7H-pyrrolo[2,3-d]pyrimidin-7-yl)tetrahydrofuran-3,4-diyl diacetate, G

To a solution of (4R,5R)-2-(acetoxymethyl)-5-(4-amino-5-cyano-7H-pyrrolo[2,3-d]pyrimidin-7-yl) tetrahydrofuran-3,4-diyl diacetate, F (2.5 g, 5.99 mmol) in AcOH (45 ml) and H₂O (15 ml) was added NaNO₂ (4.13 g, 59.88 mmol, 10 equiv.) in one portion. The resulting mixture was heated at 55° C. (oil bath temperature) for 6 h. The reaction mixture was cooled to ambient temperature and the solvents were removed under reduced pressure. The residue obtained was dissolved in EtOAc (50 ml) and washed with water and aqueous NaHCO₃ solution. The organic layer was separated, dried over Na₂SO₄, filtered and concentrated under vacuum to give crude compound G. The crude product G obtained was carried forward to next step without further purification. See, e.g., JOC, 1980, 45, 4056.

### Step 2: Preparation of (4R,5R)-2-(acetoxymethyl)-5-(4-chloro-5-cyano-7H-pyrrolo[2,3-d]pyrimidin-7-yl)tetrahydrofuran-3,4-diyl diacetate, 3

A mixture of (4R,5R)-2-(acetoxymethyl)-5-(5-cyano-4-hydroxy-7H-pyrrolo[2,3-d]pyrimidin-7-yl) tetrahydrofuran-3,4-diyl diacetate, G (1.1 g, 2.63 mmol) and POCl₃ (5 ml) was heated to 100-105° C. for 1 h. The reaction mixture was cooled to ambient temperature and concentrated under vacuum. The residue was cooled to 0° C., quenched with saturated aqueous NaHCO₃ solution and extracted with EtOAc (50 ml). The organic layer was separated, washed with saturated aqueous NaHCO₃ and brine. The EtOAc layer was dried (Na₂SO₄), filtered and concentrated to give crude product 3. The crude compound 3 was purified by silica gel column chromatography eluting with 0-40% MeOH/DCM mixtures. The product containing fractions were combined and concentrated under vacuum to afford 950 mg of compound 3. This material was used without further characterization.

### Examples 4 through 9

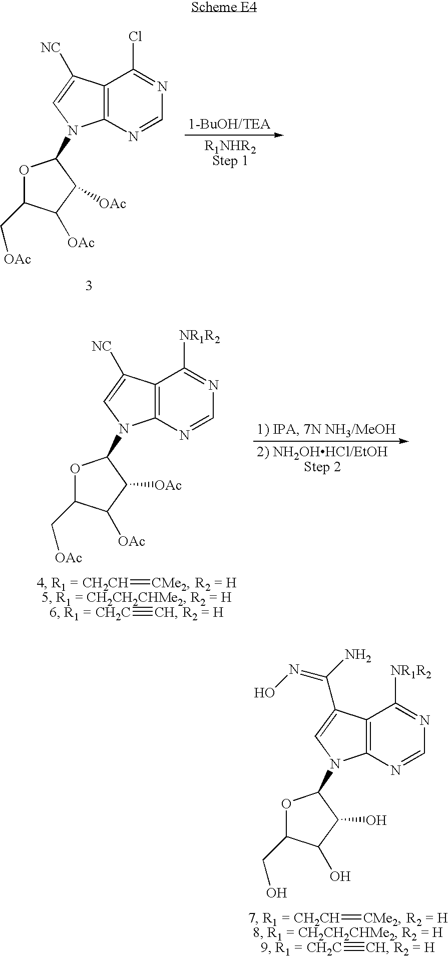

### Step 1: Preparation of (4R,5R)-2-(acetoxymethyl)-5-(5-cyano-4-(3-methylbut-2-enylamino)-7H-pyrrolo[2,3-d]pyrimidin-7-yl)tetrahydrofuran-3,4-diyl diacetate, 4

To a mixture of compound 3 (160 mg, 0.37 mmol) and anhydrous 1-BuOH (5 ml) was added triethylamine (0.52 ml, 3.67 mmol, 10 equiv.) and 3-methylbut-2-en-1-amine hydrochloride, (53.43 mg, 0.44 mmol, 1.2 equiv.). The resulting mixture was heated to 110-115° C. for 2 h. The reaction mixture was cooled to ambient temperature and the solvents were removed by evaporation under vacuum. The residue was extracted with EtOAc (25 ml), washed with 2N aqueous HCl (2×10 ml) and brine (10 ml). The organic layer was separated, dried over Na₂SO₄, filtered and concentrated to afford crude compound 4. The crude product 4 was carried to next step without further purification.

Compounds (4R,5R)-2-(acetoxymethyl)-5-(5-cyano-4-(isopentylamino)-7H-pyrrolo[2,3-d]pyrimidin-7-yl) tetrahydrofuran-3,4-diyl diacetate, 5 and (4R,5R)-2-(acetoxymethyl)-5-(5-cyano-4-(prop-2-ynylamino)-7H-pyrrolo[2,3-d]pyrimidin-7-yl)tetrahydrofuran-3,4-diyl diacetate, 6 were prepared as described above for compound 4 substituting isoamyl amine and propargyl amine for 3-methylbut-2-en-1-amine hydrochloride, respectively and were used without further purification.

### Step 2: Preparation of (Z)-7-((2R,3R)-3,4-dihydroxy-5-(hydroxymethyl)tetrahydrofuran-2-yl)-N′-hydroxy-4-(3-methylbut-2-enylamino)-7H-pyrrolo[2,3-d]pyrimidine-5-carboximidamide (compound 7)

To a solution of compound (4R,5R)-2-(acetoxymethyl)-5-(5-cyano-4-(3-methylbut-2-enylamino)-7H-pyrrolo[2,3-d]pyrimidin-7-yl)tetrahydrofuran-3,4-diyl diacetate, 4 (178 mg, 0.368 mmol) in isopropyl alcohol (4 ml) was added 7N NH₃ in MeOH solution (3.2 ml, 22.13 mmol, 60 equiv.) and the resulting mixture was stirred overnight at ambient temperature. The reaction mixture was concentrated under reduced pressure and the crude intermediate (assumed 100% conversion) was carried to next step.

To the above crude intermediate in EtOH (5 ml) was added triethylamine (0.77 ml, 5.54 mmol, 15 equiv.) and NH₂OH.HCl (256.4 mg, 3.69 mmol, 10 equiv.) and the mixture was heated at 90° C. for 14 h. The mixture was cooled to ambient temperature and concentrated under vacuum. The residue was extracted with EtOAc (50 ml) and washed with water and brine. The organic layer was separated, dried over Na₂SO₄, filtered and concentrated to give crude compound 7. The crude product was further purified by trituration with DCM and CHCl₃ to afford 45 mg of compound 7 with 95% purity (27% yield, over 3 steps). H¹ NMR (300 MHz, DMSO-d6): 10.03 (s, 1H), 9.63 (s, 1H), 8.32 (s, 1H), 8.10 (s, 1H), 7.84 (s, 1H), 6.01 (s, 2H), 5.35 (brs, 2H), 5.15 (s, 1H), 4.35 (d, 2H), 4.04 (brs, 3H), 3.89 (s, 1H), 3.54 (dd, 2H), 1.7 (s, 6H). MS: 393.2 (M+1)

Following the above experimental procedure, (Z)-7-((2R,3R)-3,4-dihydroxy-5-(hydroxymethyl) tetrahydrofuran-2-yl)-N′-hydroxy-4-(isopentylamino)-7H-pyrrolo[2,3-d]pyrimidine-5-carboximidamide, 8 and (Z)-7-((2R,3R)-3,4-dihydroxy-5-(hydroxymethyl) tetrahydrofuran-2-yl)-N′-hydroxy-4-(prop-2-ynylamino)-7H-pyrrolo[2,3-d]pyrimidine-5-carboximidamide, 9 were synthesized using (4R,5R)-2-(acetoxymethyl)-5-(5-cyano-4-(isopentylamino)-7H-pyrrolo[2,3-d]pyrimidin-7-yl)tetrahydrofuran-3,4-diyl diacetate, 5 and (4R,5R)-2-(acetoxymethyl)-5-(5-cyano-4-(prop-2-ynylamino)-7H-pyrrolo[2,3-d]pyrimidin-7-yl)tetrahydrofuran-3,4-diyl diacetate, 6, respectively.

Compound 8: H¹ NMR (300 MHz, DMSO-d6): 9.98 (brs, 1H), 9.61 (s, 1H), 8.09 (s, 1H), 7.83 (s, 1H), 6.00 (s, 3H), 5.76 (s, 1H), 5.37 (d, 1H), 5.14 (d, 2H), 4.36 (m, 1H), 4.09 (d, 1H), 3.88 (s, 1H), 3.6-3.5 (m, 3H), 1.67-1.55 (m, 3H), 0.03 (d, 6H). MS: 395.2 (M+1).

Compound 9: H¹ NMR (300 MHz, DMSO-d6): 10.32 (brs, 1H), 9.68 (s, 1H), 8.17 (s, 1H), 7.89 (s, 1H), 6.06 (s, 3H), 5.37 (d, 1H), 5.17-5.09 (m, 2H), 4.34-4.26 (m, 3H), 4.09 (s, 1H), 3.89 (s, 1H), 3.60 (dd, 2H), 3.15 (m, 1H). MS: 363.1 (M+1).

### Example 10

### Preparation of (E)-7-(2R,3R)-3,4-dihydroxy-5-(hydroxymethyl)tetrahydrofuran-2-yl)-4-(isopentylamino)-N′-methoxy-7H-pyrrolo[2,3-d]pyrimidine-5-carboximidamide, 10

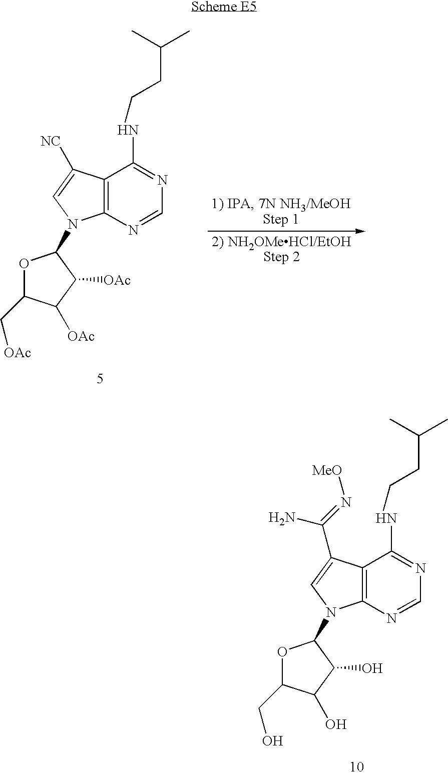

### Step 1: Preparation of 7-((2R,3R)-3,4-dihydroxy-5-(hydroxymethyl)tetrahydrofuran-2-yl)-4-(isopentylamino)-7H-pyrrolo[2,3-d]pyrimidine-5-carbonitrile

To a solution of compound (4R,5R)-2-(acetoxymethyl)-5-(5-cyano-4-(isopentylamino)-7H-pyrrolo[2,3-d]pyrimidin-7-yl) tetrahydrofuran-3,4-diyl diacetate, 5 (244.8 mg, 0.6 mmol) in IPA (6 ml) was added 7N NH₃ in MeOH solution (5.14 ml, 36 mmol, 60 equiv.) and the mixture was stirred overnight at ambient temp. The mixture was concentrated under vacuum to give crude intermediate 7-((2R,3R)-3,4-dihydroxy-5-(hydroxymethyl)tetrahydrofuran-2-yl)-4-(isopentylamino)-7H-pyrrolo[2,3-d]pyrimidine-5-carbonitrile. This material was used without further purification.

### Step 2: Preparation of (E)-7-((2R,3R)-3,4-dihydroxy-5-(hydroxymethyl)tetrahydrofuran-2-yl)-4-(isopentylamino)-N′-methoxy-7H-pyrrolo[2,3-d]pyrimidine-5-carboximidamide, 10

To the above crude intermediate was added EtOH (10 ml), triethylamine (1.256 ml, 9 mmol, 15 equiv.) and methoxyamine hydrochloride (501.12 mg, 6 mmol, 10 equiv.) and the mixture was heated at 90° C. for 36 h. The reaction mixture was cooled to ambient temperature and concentrated under vacuum to give crude product 10. The crude product was purified by passing through a silica gel column eluting with 0-30% MeOH/DCM mixture. The product containing fractions were combined and concentrated to give 21 mg of compound 10 with >97% purity (8.5% yield, over 2 steps). H¹ NMR (300 MHz, DMSO-d₆): 9.75 (t, 1H), 8.11 (s, 1H), 7.90 (s, 1H), 6.27 (S, 2H), 6.04 (d, 1H), 5.35 (d, 1H), 5.35-5.07 (m, 2H), 4.33 (q, 1H), 4.33 (dd, 1H), 4.09 (dd, 1H), 3.89 (dd, 1H), 3.80 (s, 3H), 3.6-3.48 (m, 3H), 1.7-1.685 (m, 1H), 1.56-1.49 (m, 2H), 0.93 (d, 6H). MS: 409.2 (M+1).

### Example 11

### (2R,3R,4R,5R)-2-(4-amino-5-carbamothioyl-7H-pyrrolo[2,3-d]pyrimidin-7-yl)-5-(isobutyryloxymethyl)tetrahydrofuran-3,4-diyl bis(2-methylpropanoate), 11

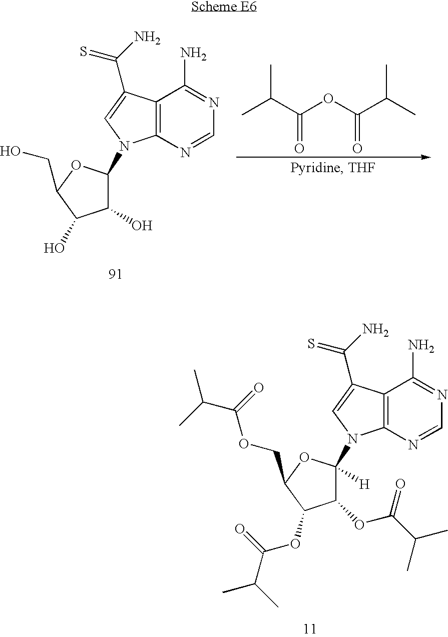

### Preparation of 11: (2R,3R,4R,5R)-2-(4-amino-5-carbamothioyl-7H-pyrrolo[2,3-d]pyrimidin-7-yl)-5-(isobutyryloxymethyl)tetrahydrofuran-3,4-diylbis(2-methylpropanoate), 11

To a suspension of 91 (0.1 g, 0.31 mmol, 1 equiv.) in pyridine was added isobutyric anhydride (0.16 mL, 0.96 mmol, 3.1 equiv.). The reaction was stirred at room temperature for 2 hours. Due to poor solubility of 91 in pyridine, there was no reaction. Tetrahydrofuran (15 mL) was added and the reaction was stirred at room temperature for 24 hours. There was no reaction due to poor solubility. The reaction mixture was heated at 50° C. for 24 hours. The reaction mixture became clear solution. LC-MS showed the formation of mono, di, tri and tetra acylated products (tri-acylated product was the major product). The reaction was stopped and the volatiles were removed in vacuo. Column purification using 0-50% ethyl acetate/dichloromethane afforded 120 mg of pure 11 (75% yield, >99% purity by LC-MS and ¹H-NMR) as a yellow oil. ¹H NMR (300 MHz, CDCl₃): δ 8.23 (s, 1H), 7.82 (bs, 2H), 7.73 (s, 1H), 7.43 (s, 2H), 6.49 (d, 1H), 5.64 (t, 1H), 5.48 (dd, 1H), 4.58 (dd, 1H), 4.36-4.47 (m, 1H), 4.29 (dd, 1H), 2.47-2.72 (m, 3H), 1.06-1.30 (m, 18H); MS: 536.1 (M+1); calcd for C₂₄H₃₃N₅O₇S: 535.21.

### Example 12

### (2R)-3-(hexadecyloxy)propyl 2-((((3S,4R,5R)-5-(4-amino-5-cyano-7H-pyrrolo[2,3-d]pyrimidin-7-yl)-3,4-dihydroxytetrahydrofuran-2-yl)methoxy)(phenoxy) phosphorylamino)propanoate, 17

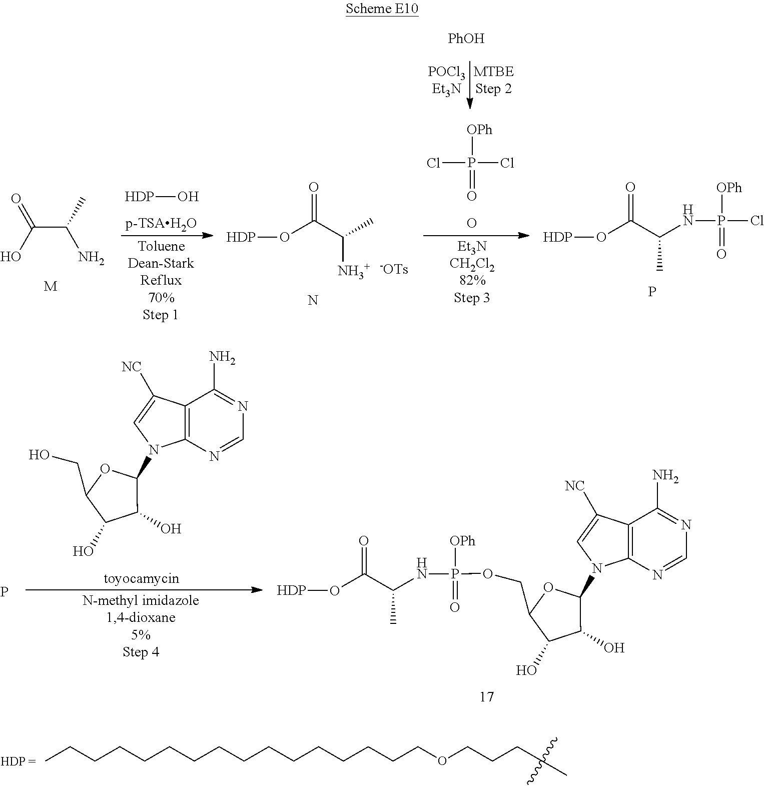

### Step 1: Preparation of (S)-3-(hexadecyloxy)propyl 2-aminopropanoate tosylate, N

L-alanine (25 g, 280.6 mmol, 1 equiv.) was suspended in toluene (700 mL), p-toluenesulfonic acid (58.7 g, 308.7 mmol, 1.1 equiv.) and HDP-OH (169 g, 561.2 mmol, 2 equiv.), were added and the resulting mixture was heated at reflux with Dean-Stark trap overnight. The reaction mixture was evaporated in vacuo. The crude solid was triturated in hexanes, filtered and dried to afford 130 g (85% yield) of slightly impure N as the tosylate salt. This material was used without further purification.

### Step 2: Preparation of phenyl phosphorodichloridate, O

To a solution of phenol (18.8 g, 200 mmol, 1 equiv.) in MTBE (350 mL), POCl₃ (18.6 mL, 200 mmol, 1 equiv.) was gradually added and the mixture was cooled to −55° C. Et₃N (27.9 mL, 200 mmol, 1 equiv.) was slowly added. After one hour, the reaction mixture was allowed to warm to room temperature and the mixture was stirred for 3 h. The reaction mixture was filtered and the filtrate was evaporated in vacuo at 20° C. to afford 41.2 g (98% yield) of crude O as a light yellow liquid. The yellow liquid was used in the next step without purification.

### Step 3: Preparation of (2S)-3-(hexadecyloxy)propyl 2-(chloro(phenoxy)phosphorylamino)propanoate, P

To a solution of (S)-3-(hexadecyloxy)propyl 2-aminopropanoate tosylate, N (15.7 g, 28.9 mmol, 1 equiv.) and phenyl phosphorodichloridate, O (6.1 g, 28.9 mmol, 1 equiv.) in CH₂Cl₂ (100 mL) at −25° C., Et₃N (8.1 mL, 57.8 mmol, 2 equiv.) was slowly added. After one hour, the reaction mixture was allowed to warm to room temperature and the mixture was stirred overnight. The reaction mixture was evaporated in vacuo, the residue was triturated in EtOAc and filtered to remove triethylamine hydrochloride. Evaporation of filtrate followed by column chromatography (0-40% EtOAc/hexanes) afforded 13 g of pure P (82% yield) as a colorless oil.

### Step 4: Preparation of (2R)-3-(hexadecyloxy)propyl 2-((((3S,4R,5R)-5-(4-amino-5-cyano-7H-pyrrolo[2,3-d]pyrimidin-7-yl)-3,4-dihydroxytetrahydrofuran-2-yl)methoxy) (phenoxy) phosphorylamino) propanoate, 17

To a suspension of 4-amino-7-((2R,3R,4S)-3,4-dihydroxy-5-(hydroxymethyl)tetrahydrofuran-2-yl)-7H-pyrrolo[2,3-d]pyrimidine-5-carbonitrile (toyocamycin) (1.17 g, 4 mmol, 1 equiv.) in 1,4-dioxane (40 mL), N-methyl imidazole (1.9 mL, 23.8 mmol, 6 equiv.) was added at room temperature to effect a clear solution. (2S)-3-(hexadecyloxy)propyl 2-(chloro(phenoxy)phosphorylamino)propanoate, P (6.5 g, 11.9 mmol, 3 equiv.) in 1,4-dioxane (20 mL) was added dropwise at room temperature to form a turbid solution. After 2.5 days, LC-MS showed ˜15 to 20% conversion to 17 and formation of the free base of N as a by-product. The reaction mixture was concentrated in vacuo. The residue was dissolved in EtOAc, washed with 0.5 N HCl, saturated NaHCO₃, water, brine, dried (Na₂SO₄) and evaporated in vacuo. Column chromatography of the residue (silica, packed with 0.4% AcOH/EtOAc) by elution with 0-5% THF/EtOAc containing 0.4% AcOH afforded 0.25 g of impure 17 contaminated with AcOH, BHT from THF, and a trace of free base of 2. This was dissolved in EtOAc, washed with saturated NaHCO₃, water, brine, dried (Na₂SO₄) and evaporated in vacuo to afford 0.183 g (5% yield) of slightly impure 17 as an yellow foaming solid contaminated with BHT from THF, trace of free base of 2 and an unidentified minor impurity in ¹H-NMR. ³¹P-NMR of 17 showed 4 peaks. However, ³¹P-NMR of P showed only 2 peaks. Possibly epimerization happened during this reaction. H¹ NMR (300 MHz, DMSO-d₆): 8.89 (d, 2H), 8.43 (s, 2H), 8.34 (s, 1H), 8.00 (m, 1H), 6.13 (d, 1H), 4.37 (m, 1H), 4.10 (m, 4H), 3.92 (m, 2H), 3.33 (m, 4H), 1.78 (m, 2H), 1.43 (m, 2H), 1.23 (m, 24H), 0.85 (m, 3H). MS: 654.3 (M+1).

### Example 13

### (2R)-3-(hexadecyloxy)propyl 2-((((3S,4R,5R)-5-(4-amino-5-carbamothioyl-7H-pyrrolo[2,3-d]pyrimidin-7-yl)-3,4-dihydroxytetrahydrofuran-2-yl)methoxy)(phenoxy)phosphorylamino)propanoate, 18

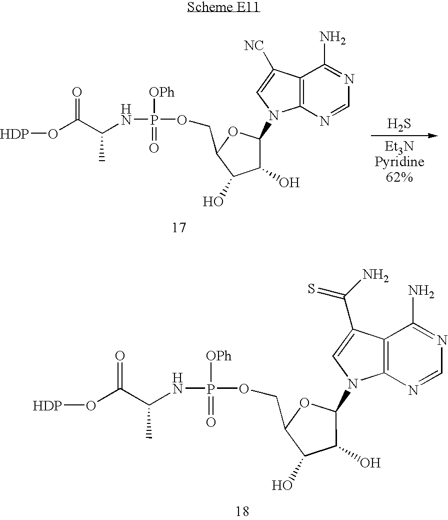

Slightly impure 17 (0.18 g, 0.22 mmol, 1 equiv.) was dissolved in pyridine (5 mL) and Et₃N (0.2 mL, 1.1 mmol, 6 equiv.) was added. H₂S gas was bubbled through this solution at 0° C. to room temp. for 4 h. LC-MS showed completion of reaction. Volatiles were removed in vacuo and the residue was loaded on a column (silica, packed with 0.4% AcOH/CH₂Cl₂). Elution with 0-4% MeOH/CH₂Cl₂ containing 0.4% AcOH afforded two sets of fractions. Careful TLC analysis revealed very slight differences in Rf value between these fractions. Component A, the Less polar major fractions afforded 110 mg of 18 (after saturated NaHCO₃ work up to remove traces of AcOH) as a mixture of two sets of diastereomers (less polar:more polar ˜4:3 ratio based on ³¹P-NMR). LC-MS showed single component broad peak m/e corresponding to 18 (retention time: 5.34). Component B, the more polar minor fractions afforded 6 mg of 18 as a yellow solid (after saturated NaHCO₃ work up to remove traces of AcOH) and as a mixture of (less polar:more polar ˜1:5 ratio based on ³¹P-NMR) two sets of diastereomers. LC-MS showed single component relatively sharper peak m/e corresponding to 18 (retention time: 5.39, >95% purity).

Component A, the 110 mg of 18 from above was purified using the Biotage flash purification system eluting with 2.5%-5% MeOH/CH₂Cl₂. 7 mgs of a mixture of (less polar:more polar ˜6:1 ratio based on ³¹P-NMR, >95% purity by LC-MS) two sets of diastereomers were obtained.

Other fractions only yielded 64 mg of a mixture of two sets of diastereomers, >95% purity by LC-MS), ¹H NMR (300 MHz, CD₃OD): δ 8.12 (s, 1H), 7.87 (m 1H), 7.10-7.38 (m, 5H), 6.24 (m, 1H), 4.19-4.50 (m, 5H), 4.00-4.18 (2H), 3.75-3.99 (m, 1H), 3.24-3.48 (m, 6H), 1.73-1.90 (m, 2H), 1.40-1.56 (m, 2H), 1.27 (s, 32H), 0.89 (t, 3H); ³¹P NMR: δ 3.90, 3.74, 3.53, 3.34; MS: 835.5 (M+1); calcd for C₄₀H₆₃N₆O₉PS: 834.41.

### Examples 14 and 15

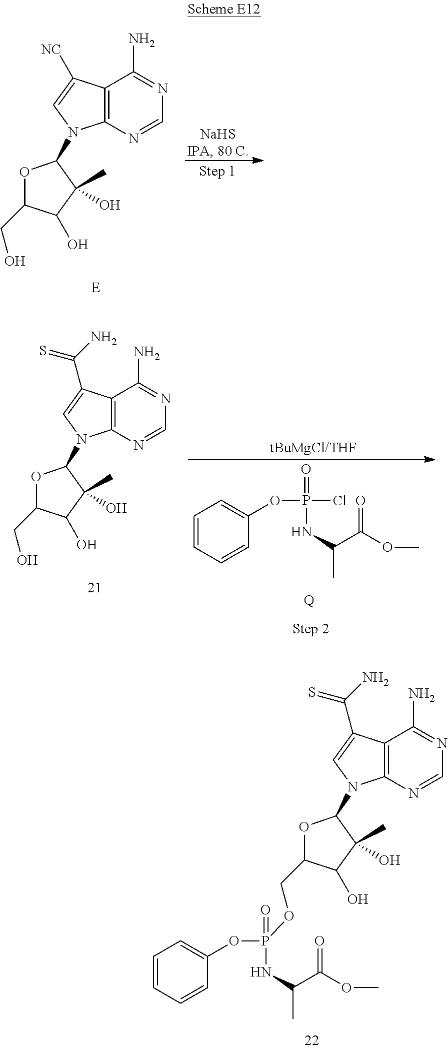

Step 1, Example 14: Preparation of 4-amino-7-((2R,3R)-3,4-dihydroxy-5-(hydroxymethyl)-3-methyl tetra hydro furan-2-yl)-7H-pyrrolo[2,3-d]pyrimidine-5-carbothioamide, 21.

A mixture of 4-amino-7-((2R,3R)-3,4-dihydroxy-5-(hydroxymethyl)-3-methyltetrahydrofuran-2-yl)-7H-pyrrolo[2,3-d]pyrimidine-5-carbonitrile, E with its methanol adduct (1.1 g, 1.78 mmol), and NaHS (0.5 g, 8.9 mmol) in isopropanol (10 ml) was stirred at 80° C. for 18 h. The reaction mixture was concentrated to dryness. The residue was suspended in methanol/water (1:2, 30 ml) and filtered. The filtered solid was washed with water (20 ml), and dried under vacuum. The resulting white solid was washed with CH₂Cl₂ (2×20 ml) and dried under vacuum to give 485 mg, (80%) of 4-amino-7-((2R,3R)-3,4-dihydroxy-5-(hydroxymethyl)-3-methyltetrahydrofuran-2-yl)-7H-pyrrolo[2,3-d]pyrimidine-5-carbothioamide, 21 as a yellow solid. ¹H NMR (DMSO-d₆) δ (ppm) 9.54 (br d, 2H), 8.13 (s, 1H), 7.94 (s, 1H), 7.9 (br s, 2H), 6.2 (s, 1H), 5.2 (s, 1H), 5.08-5.02 (m, 2H), 4.02-3.63 (m, 4H), 0.75 (s, 3H).

Step 2, Example 15: Preparation of (2S)-methyl 2-((((4R,5R)-5-(4-amino-5-carbamothioyl-7H-pyrrolo[2,3-d]pyrimidin-7-yl)-3,4-dihydroxy-4-methyltetrahydrofuran-2-yl)methoxy)(phenoxy)phosphorylamino)propanoate, 22.

In a 40 mL vial, 4-amino-7-((2R,3R)-3,4-dihydroxy-5-(hydroxymethyl)-3-methyl tetrahydro furan-2-yl)-7H-pyrrolo[2,3-d]pyrimidine-5-carbothioamide, 21 (477 mg, 1.4 mmol) was suspended in 15 mL THF; and cooled to 0° C.; t-BuMgCl solution (3.5 mL, 3.5 mmol) was slowly added. The reaction mixture was allowed to stir for 30 min., then a solution of (2S)-methyl 2-(chloro(phenoxy)phosphorylamino)propanoate, Q (800 mg, 2.2 mmol) in THF (7 mL) was gradually added. After one hour the mixture was allowed to warm to room temperature and was stirred at room temperature for 27 hours. The reaction mixture was quenched with saturated aqueous ammonium chloride. After stirring for 10 minutes, the mixture was extracted with ethyl acetate (2×20 mL). The combined organic phase was washed with brine (2×10 mL) and dried over sodium sulfate. The solvents were removed in vacuo and the residue was loaded on a column. Elution with dichloromethane/MeOH (9/1) gave 190 mg (23%) of (2S)-methyl 2-((((4R,5R)-5-(4-amino-5-carbamothioyl-7H-pyrrolo[2,3-d]pyrimidin-7-yl)-3,4-dihydroxy-4-methyltetrahydrofuran-2-yl)methoxy)(phenoxy)phosphorylamino)propanoate, 22. ¹H NMR (DMSO-d₆) δ (ppm) 9.66 (br 1, 1H), 9.52 (s, 1H), 8.16 (s, 1H), 8.06 (br s, 2H), 7.70 (s, 1H), 7.42-7.10 (m, 5H), 6.14 (s, 1H), 6.08 (m, 1H), 5.38 (d, 1H), 5.37 (s, 1H), 4.45-3.75 (m, 5H), 3.58, 3.50 (2 s, 3H), 3.18 (d, J=8 Hz, 1H), 1.20 (d, 3H), and 0.75 (s, 3). ³¹P NMR at RT (dmso-d₆) 6 (ppm) 3.68 (s, int=2), 3.82 (s, int=1); ³¹P NMR at 80° C. (dmso-d₆) δ (ppm) 3.50 (s). LC-MS: (M+1) 581.

### Example 16

### Preparation of (2S)-methyl 2-((((4R,5R)-5-(4-amino-5-carbamothioyl-7H-pyrrolo[2,3-d]pyrimidin-7-yl)-3,4-dihydroxytetrahydrofuran-2-yl)methoxy)(phenoxy)phosphoryl amino)propanoate, 23

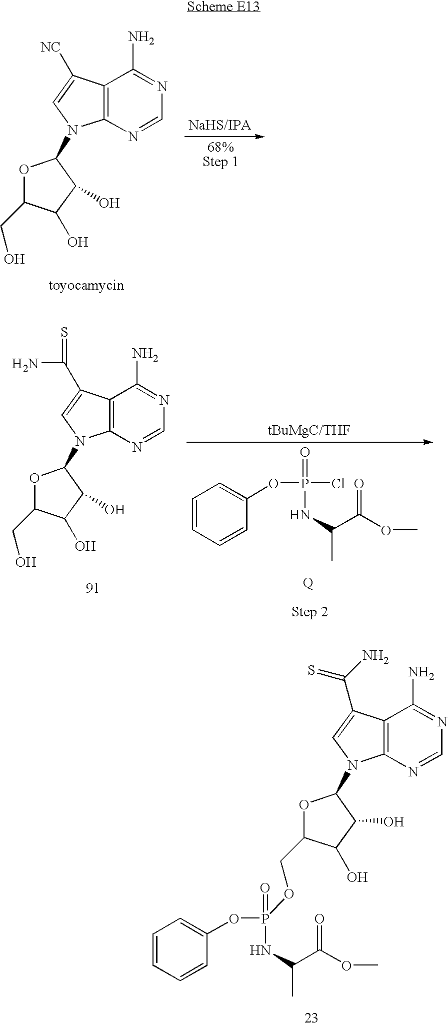

### Step 1: Preparation of 4-amino-7-((2R,3R)-3,4-dihydroxy-5-(hydroxymethyl)tetrahydro furan-2-yl)-7H-pyrrolo[2,3-d]pyrimidine-5-carbothioamide, 91

Toyocamycin (2.91 g, 10 mmol) was taken up in anhydrous isopropanol (50 ml) in an inert atmosphere and anhydrous sodium hydrosulfide hydrate (1.4 g, 25 mmol) was added. The reaction mixture was heated at 60-80° C. for 24 h and LC/MS indicated 80% conversion to product. The solvent was removed under reduced pressure and the solid was passed through a silica column eluting with dichloromethane/methanol (8:2) to give 2.2 g (68%) of 4-amino-7-((2R,3R)-3,4-dihydroxy-5-(hydroxymethyl)tetrahydrofuran-2-yl)-7H-pyrrolo[2,3-d]pyrimidine-5-carbothioamide, 91 as a light yellow solid.

### Step 2, Example 16: Preparation of (2S)-methyl 2-((((4R,5R)-5-(4-amino-5-carbamothioyl-7H-pyrrolo[2,3-d]pyrimidin-7-yl)-3,4-dihydroxytetrahydrofuran-2-yl)methoxy)(phenoxy)phosphoryl amino)propanoate, 23

In a 40 mL vial, 4-amino-7-((2R,3R)-3,4-dihydroxy-5-(hydroxymethyl)tetrahydrofuran-2-yl)-7H-pyrrolo[2,3-d]pyrimidine-5-carbothioamide, 91 (370 mg, 1.1 mmol) was suspended in THF (15 mL) and cooled to 0° C. t-BuMgCl solution (2.5 mL, 2.5 mmol) was slowly added. The reaction mixture was allowed to stir for 30 min., then a solution of (2S)-methyl 2-(chloro(phenoxy)phosphorylamino)propanoate Q (660 mg, 2.2 mmol) in THF (7 mL) was gradually added. After one hr., the mixture was allowed to warm up to room temperature and was stirred for 73 hr. The reaction mixture was quenched with saturated aqueous ammonium chloride. After stirring for 10 min, the mixture was extracted with ethyl acetate (2×20 mL). The combined organic phase was washed with brine (2×10 mL) and dried over sodium sulfate. The solvent was removed in vacuo and the residue was loaded on a silica gel column. Elution with dichloromethane/MeOH (9/1) gave 50 mg (8%) of (2S)-methyl 2-((((4R,5R)-5-(4-amino-5-carbamothioyl-7H-pyrrolo[2,3-d]pyrimidin-7-yl)-3,4-dihydroxy tetra hydrofuran-2-yl)methoxy)(phenoxy)phosphorylamino)propanoate, 23. ¹H NMR (CD₃OD) 8 (ppm) 8.15 (s 1, 1H), 7.81 (2s, 1H), 7.42-7.10 (m, 5H), 6.24 (m, 1H), 4.45-3.75 (m, 6H), 3.68, 3.61 (2 s, 3H), 1.22 (d, 3H). LC-MS: (M+1) 567

### Examples 17 and 18

### ((2R,3R,4R,5R)-5-(4-amino-5-cyano-7H-pyrrolo[2,3-d]pyrimidin-7-yl)-3,4-dihydroxy-4-methyltetrahydrofuran-2-yl)methyl 3-(hexadecyloxy)propyl hydrogen phosphate, 24 and [4-amino-7-((2R,3R,4R,5R)-3,4-dihydroxy-5-(hydroxymethyl)-3-methyltetrahydrofuran-2-yl)-7H-pyrrolo[2,3-d]pyrimidine-5-carbonitrile]-3′,5′-(3-(hexadecyloxy)propyl)cyclic phosphate, 25.

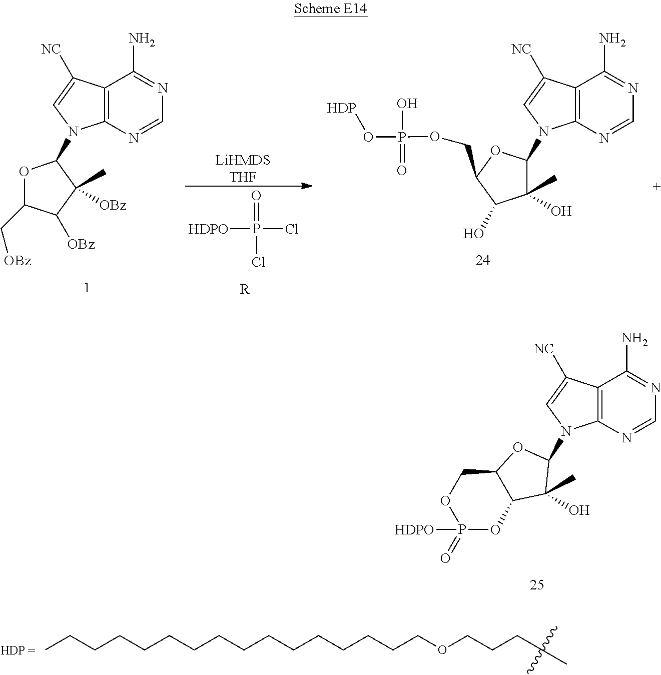

A solution of (2R,3R)-2-(4-amino-5-cyano-7H-pyrrolo[2,3-d]pyrimidin-7-yl)-5-(benzoyloxymethyl)-3-methyltetrahydrofuran-3,4-diyl dibenzoate, 1 (306 mg, 1.0 mmol) in THF (10 ml) was added lithium hexamethyldisilazane (2 ml, 2 mmol, 1M solution in THF). The mixture was stirred for 15 min at room temp. and then 3-(hexadecyloxy)propyl phosphorodichloridate, R (458 mg, 1.1 mmol) was added. The mixture was stirred at room temp for 16 h. The LC-MS analysis indicated a mixture of uncyclized phosphate 24 (major) and cyclized phosphate 25 (˜2%). The mixture was concentrated to dryness in vacuo. The residue was treated with water (50 ml) and extracted with CHCl₃ (2×100 ml). The organics were dried over Na₂SO₄ and concentrated to afford a yellow residue. Column chromatography on silica eluting with CHCl₃/MeOH (98:2) afforded the cyclic phosphate 25 with minor impurities (120 mg), then eluting with CHCl₃/MeOH (70:30) gave the uncyclized phosphate 24 with some impurities (380 mg). Both compounds 24 and 25 were used without further purification.

### Example 19

### Preparation of ((2R,3R,4R,5R)-5-(4-amino-5-carbamothioyl-7H-pyrrolo[2,3-d]pyrimidin-7-yl)-3,4-dihydroxy-4-methyltetrahydrofuran-2-yl)methyl 3-(hexadecyloxy)propyl hydrogen phosphate, 26

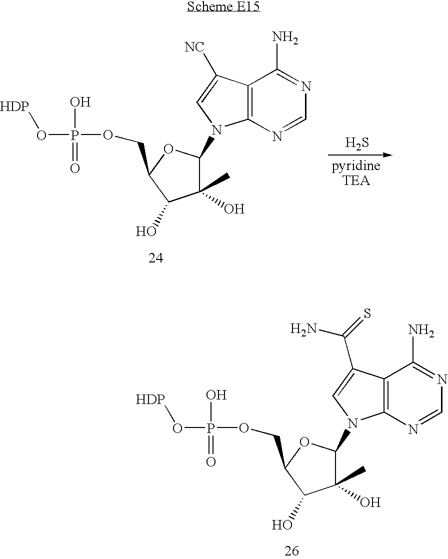

A solution of ((2R,3R,4R,5R)-5-(4-amino-5-cyano-7H-pyrrolo[2,3-d]pyrimidin-7-yl)-3,4-dihydroxy-4-methyltetrahydrofuran-2-yl)methyl 3-(hexadecyloxy)propyl hydrogen phosphate, 24 (180 mg, 0.27 mmol) in a mixture of pyridine and triethylamine (12 ml. 5:1) was cooled to 0° C. A stream of H₂S (gas) was passed through the solution for 45 min. The solution became green and formed a suspension. The cooling bath was removed and the mixture was stirred at room temp for 1 h. The mixture was concentrated to dryness in vacuo. The orange residue was dissolved in CHCl₃ and loaded onto a silica gel column packed with CHCl₃. The initial purification eluting with 98:2→70:30 CHCl₃/MeOH afforded a product with a single peak on HPLC having a mass corresponding to the required product 26. However ¹H NMR and ³¹P-NMR revealed two products in a 2:1 ratio. A second gravity column eluting with 98:2→70:30 CHCl₃/MeOH afforded three components. Component A, the less polar product 26 (14 mg, 7.4%, ˜90% pure), Component B, as a mixture of more and less polar product 26 in 2:1 ratio (16 mg, 8.5%) and Component C, a mixture of more and less polar products with an ˜1:1 ratio (40 mg, 21%) was isolated. Data for Component A, compound 26: ¹H NMR (CD₃OD): δ 8.26 (s, 1H), 8.21 (s, 1H), 6.32 (s, 1H), 4.65 (t, 1H), 4.18 (d, 1H), 3.9 (m, 4H), 3.55 (m, 2H), 3.51 (m, 2H), 1.88 (t, 2H), 1.53 (t, 2H), 1.23 (m, 30H), 0.89 (m, 6H); ³¹P NMR (CD₃OD): 0.61 (s); LC-MS: 702.4 (M+1).

### Example 20

### Preparation of [4-amino-7-((2R,3R,4R,5R)-3,4-dihydroxy-5-(hydroxymethyl)-3-methyltetrahydrofuran-2-yl)-7H-pyrrolo[2,3-d]pyrimidine-5-carbothioamide]-3′,5′-(3-(hexadecyloxy)propyl)cyclic phosphate, 27

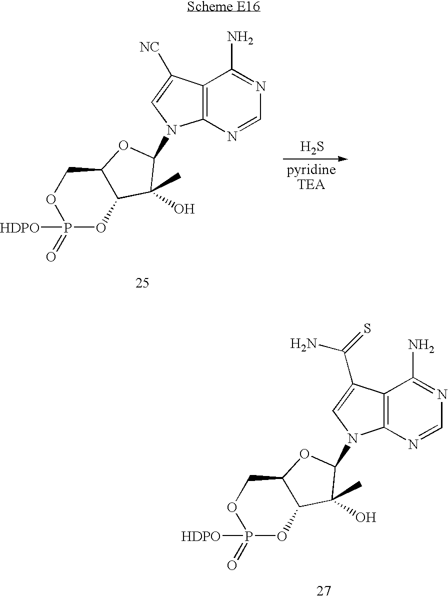

A solution of [4-amino-7-((2R,3R,4R,5R)-3,4-dihydroxy-5-(hydroxymethyl)-3-methyltetrahydrofuran-2-yl)-7H-pyrrolo[2,3-d]pyrimidine-5-carbonitrile]-3′,5′-(3-(hexadecyloxy)propyl)cyclic phosphate, 25 (120 mg, 0.18 mmol) in a mixture of pyridine and triethylamine (12 ml. 5:1) was cooled to 0° C. A stream of H₂S (gas) was passed through the solution for 45 min. The solution became green and formed a suspension. The cooling bath was removed and the mixture was stirred at room temperature for 1 h. LC-MS analysis indicated complete reaction and revealed 2 products with a mass corresponding to compound 27 in 1:1 ratio. The mixture was concentrated to dryness. The orange residue was dissolved in CHCl₃ and loaded onto a column packed with silica/CHCl₃. The column was eluted with 98:2 CHCl₃/MeOH to give two components. Component A, the less polar product 27 (7.5 mg, 6%) and Component B, the impure more polar product 27 (19 mg, 15%, ˜50% pure). Data for Component A, 27: ¹H NMR (CD₃OD): δ 8.15 (s, 1H), 7.75 (s, 1H), 6.4 (br, 1H), 4.75 (m, 3H), 4.2 (m, 2H), 3.55 (m, 2H), 3.43 (m, 2H), 1.92 (m, 2H), 1.57 (m, 2H), 1.38 (m, 30H), 0.96 (m, 6H); ³¹P NMR (CD₃OD): −3.54 (s); LC-MS: 684.4 (M+1).

### Examples 21, 22 and 23

### Preparation of 7-((2R,3R)-3,4-dihydroxy-5-(hydroxymethyl)tetrahydrofuran-2-yl)-4-(isopentylamino)-7H-pyrrolo[2,3-d]pyrimidine-5-carbonitrile, 28; ((4R,5R)-5-(5-cyano-4-(isopentylamino)-7H-pyrrolo[2,3-d]pyrimidin-7-yl)-3,4-dihydroxytetrahydrofuran-2-yl)methyl 3-(hexadecyloxy)propyl hydrogen phosphate, 29 and ((4R,5R)-3,4-dihydroxy-5-(5-(N′-hydroxy carbamimidoyl)-4-(isopentylamino)-7H-pyrrolo[2,3-d]pyrimidin-7-yl)tetrahydrofuran-2-yl)methyl 3-(hexadecyloxy)propyl hydrogen phosphate, 30.

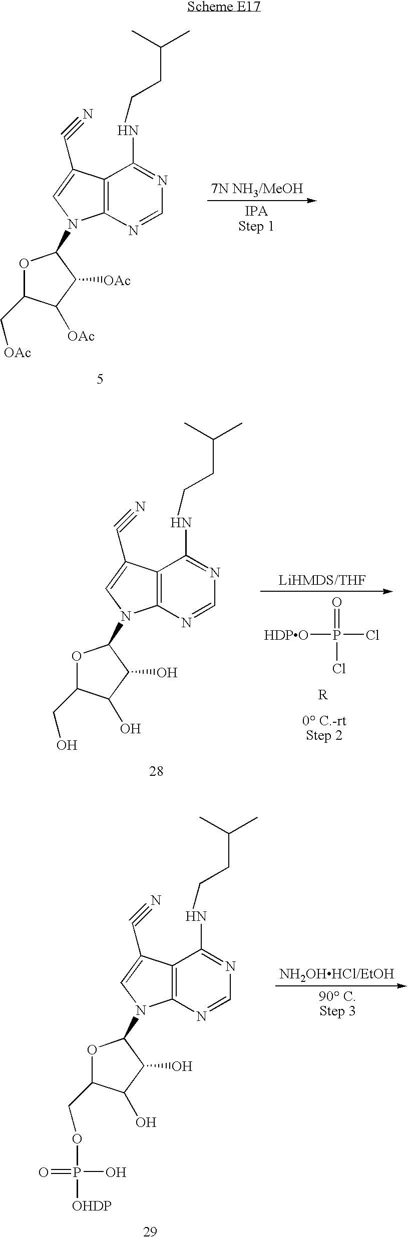

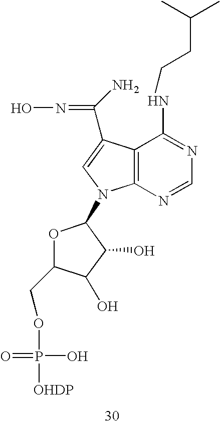

### Step 1: Preparation of 7-((2R,3R)-3,4-dihydroxy-5-(hydroxymethyl)tetrahydrofuran-2-yl)-4-(isopentylamino)-7H-pyrrolo[2,3-d]pyrimidine-5-carbonitrile, 28

See Scheme E4, Step 2, first reaction.

### Step 2: Preparation of ((4R,5R)-5-(5-cyano-4-(isopentylamino)-7H-pyrrolo[2,3-d]pyrimidin-7-yl)-3,4-dihydroxytetrahydrofuran-2-yl)methyl 3-(hexadecyloxy)propyl hydrogen phosphate, 29

A mixture of 7-((2R,3R)-3,4-dihydroxy-5-(hydroxymethyl)tetrahydrofuran-2-yl)-4-(isopentylamino)-7H-pyrrolo[2,3-d]pyrimidine-5-carbonitrile, 5 (70 mg, 0.2 mmol) and THF (10 ml) was cooled to 0° C. and 1M LiHMDS in THF (0.48 ml, 0.48 mmol, 2.5 equiv.) was added. The mixture was stirred at 0° C. for 20 min then warmed to ambient temperature and stirred for 30 min. The mixture was then cooled to 0° C. and 3-(hexadecyloxy)propyl phosphorodichloridate, compound R (96.8 mg, 0.23 mmol, 1.2 equiv.) was added and the mixture was warmed to ambient temperature and stirred overnight. The reaction was quenched by addition of saturated aqueous NH₄Cl solution and the mixture was extracted with EtOAc (25 ml). The organic layer was separated, washed with brine, dried over Na₂SO₄ filtered and concentrated under vacuum to give crude compound 29. The crude product 29 was purified by silica gel column chromatography eluting with 0-50% MeOH/DCM mixtures to give 20 mg of compound 29 (14.2% yield, >90% purity). This product was carried forward to final step without further purification.

### Step 3: Preparation of ((4R,5R)-3,4-dihydroxy-5-(5-(N′-hydroxycarbamimidoyl)-4-(isopentylamino)-7H-pyrrolo[2,3-d]pyrimidin-7-yl)tetrahydrofuran-2-yl)methyl 3-(hexadecyloxy)propyl hydrogen phosphate, 30

To a mixture of ((4R,5R)-5-(5-cyano-4-(isopentylamino)-7H-pyrrolo[2,3-d]pyrimidin-7-yl)-3,4-dihydroxy tetrahydrofuran-2-yl)methyl 3-(hexadecyloxy)propyl hydrogen phosphate, 29 (20 mg, 0.28 mmol), EtOH (10 ml) and triethylamine (0.77 ml, 0.55 mmol, 20 equiv.) was added hydroxylamine hydrochloride (19.2 mg, 0.28 mmol, 10 equiv.) at ambient temperature. The mixture was heated at 90° C. for 18 h. The reaction mixture was cooled to ambient temperature and solvents were removed under vacuum. EtOAc (50 ml) was added to the residue and the mixture was washed with brine. The organic layer was separated, dried over Na₂SO₄, filtered and concentrated under reduced pressure to give 15 mg of compound 30 (71% yield, >96% purity). H¹ NMR (300 MHz, MeOH-d4): 8.08 (s, 1H), 7.77 (s, 1H), 6.07 (d, 1H), 4.68 (dd, 2H), 4.34 (d, 1H), 4.02 (q, 2H), 3.85-3.89 (brs, 2H), 3.56 (m, 4H), 3.39 (t, 2H), 1.911 (m, 2H), 1.89-1.87 (m, 3H), 1.77-1.72 (m, 2H), 1.27-1.25 (m, 30H), 0.99 (d, 6H), 0.89 (m, 3H). MS: 757.5 (M+1). P³¹NMR (300 MHz, MeOH-d4): 0.54 (s), −0.30 (s).

Compound 92, ((4R,5R)-3,4-dihydroxy-5-(5-(N′-hydroxycarbamimidoyl)-4-(3-methylbut-2-enylamino)-7H-pyrrolo[2,3-d]pyrimidin-7-yl)tetrahydrofuran-2-yl)methyl 3-(hexadecyloxy)propyl hydrogen phosphate is made in an analogous manner to that described for compound 30.

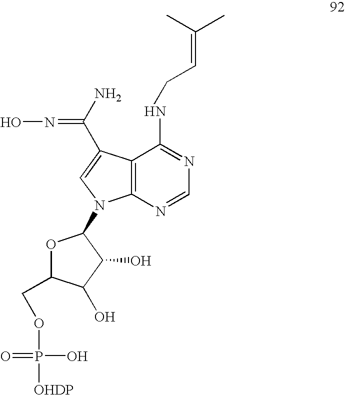

### Examples 24 and 25

### Preparation of ((2R,3S,4R,5R)-5-(4-amino-5-cyano-7H-pyrrolo[2,3-d]pyrimidin-7-yl)-3,4-dihydroxytetrahydrofuran-2-yl)methyl 3-(hexadecyloxy)propyl hydrogen phosphate, 33 and ((2R,3S,4R,5R)-5-(4-amino-5-carbamothioyl-7H-pyrrolo[2,3-d]pyrimidin-7-yl)-3,4-dihydroxy tetra hydrofuran-2-yl)methyl 3-(hexadecyloxy)propyl hydrogen phosphate, 34.

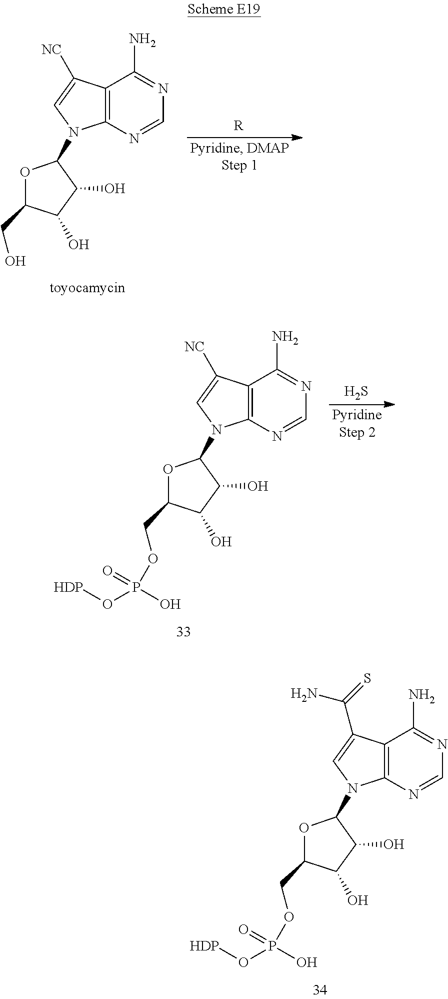

### Step 1: Preparation of ((2R,3S,4R,5R)-5-(4-amino-5-cyano-7H-pyrrolo[2,3-d]pyrimidin-7-yl)-3,4-dihydroxytetrahydrofuran-2-yl)methyl 3-(hexadecyloxy)propyl hydrogen phosphate, 33

Toyocamycin (0.146 g, 0.5 mmol) was dissolved in anhydrous pyridine (10 ml) and cooled to 0° C. and 3-(hexadecyloxy)propyl phosphorodichloridate, R (0.208 g, 0.5 mmol) was added portionwise. The reaction mixture was allowed to warm to room temperature and was stirred for 24 h. LC/MS was observed only 50-60% of product formation along with some un-reacted starting compound 33. The solvent was evaporated under reduced pressure and the residue was purified by silica gel column chromatography eluting with dichloromethane/methanol (8:2) to give 47 mg (Yield 15%) of ((2R,3S,4R,5R)-5-(4-amino-5-cyano-7H-pyrrolo[2,3-d]pyrimidin-7-yl)-3,4-dihydroxytetrahydrofuran-2-yl)methyl 3-(hexadecyloxy) propyl hydrogen phosphate, 33 as an off white solid. LC/MS: M⁺ 654.3 (C₃₁H₅₂N₅O₈P, M.W: 653.36); ¹H NMR (300 MHz, DMSO): 8.88-8.90 (d, 1H), 8.43 (s, 1H), 8.34 (s, 1H), 7.98-8.0 (d, 1H), 6.12-6.14 (d, 1H), 4.36-4.37 (m, 1H), 4.08-4.12 (m, 4H), 3.91-3.93 (m, 2H), 3.27-3.39 (m, 4H), 1.76-1.80 (m, 2H), 1.42-1.43 (m, 2H), 1.21 (s, 26H), 0.83-0.85 (m, 3H).

### Step 2: Preparation of ((2R,3S,4R,5R)-5-(4-amino-5-carbamothioyl-7H-pyrrolo[2,3-d]pyrimidin-7-yl)-3,4-dihydroxytetrahydrofuran-2-yl)methyl 3-(hexadecyloxy)propyl hydrogen phosphate, 34

((2R,3S,4R,5R)-5-(4-Amino-5-cyano-7H-pyrrolo[2,3-d]pyrimidin-7-yl)-3,4-dihydroxytetrahydrofuran-2-yl)methyl 3-(hexadecyloxy)propyl hydrogen phosphate, 33 (0.038 g, 0.058 mmol) was dissolved in anhydrous pyridine (5 ml) under an inert atmosphere and a catalytic amount of triethylamine (5 mol %) was added. A slow stream of hydrogen sulfide gas (Stainless steel lecture cylinder with T-purge valve, Cat log No: 295442-227G from Aldrich) was passed through the reaction mixture for 1 h. After completion of the reaction, the solvent was evaporated under reduced pressure and the residue was purified by silica gel column chromatography eluting with dichloromethane/methanol (6:4) to give 14 mg of ((2R,3S,4R,5R)-5-(4-amino-5-carbamothioyl-7H-pyrrolo[2,3-d]pyrimidin-7-yl)-3,4-dihydroxy tetrahydrofuran-2-yl)methyl 3-(hexadecyloxy)propyl hydrogen phosphate, 34 in 35% yield. LC/MS: M⁺ 688.4 (C₃₁H₅₄N₅O₈PS, M.W: 687.34); ¹H NMR (300 MHz, DMSO): 10.73 (s, 1H), 9.27 (s, 1H), 8.54 (s, 1H), 8.09 (d, 1H), 6.20-6.21 (m, 1H), 5.49-5.51 (m, 1H), 5.26-5.27 (m, 1H), 4.38-4.39 (m, 1H), 4.13-4.14 (m, 2H), 4.01-4.02 (m 2H), 3.63-3.65 (m, 2H), 1.67-1.69 (m, 2H), 1.42-1.43 (m, 2H), 1.21 (s, 26H), 0.83-0.85 (m, 3H). ³¹P NMR (300 MHz, DMSO): Single peak

### Example 26

### Preparation of ((2R,3S,4S,5R)-5-(4-amino-5-carbamoyl-7H-pyrrolo[2,3-d]pyrimidin-7-yl)-4-azido-3-hydroxytetrahydrofuran-2-yl)methyl 3-(hexadecyloxy)propyl hydrogen phosphate, 36

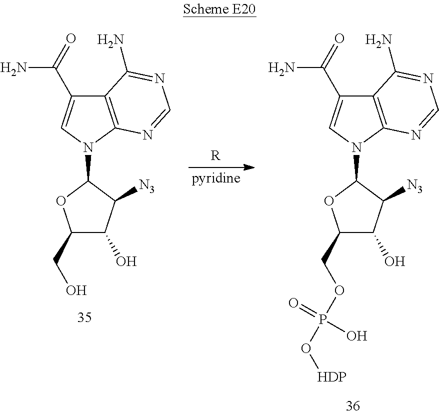

4-Amino-7-((2R,3S,4S,5R)-3-azido-4-hydroxy-5-(hydroxymethyl)tetrahydrofuran-2-yl)-7H-pyrrolo[2,3-d]pyrimidine-5-carboxamide, 35 (0.334 g, 1 mmol) was dissolved in anhydrous pyridine (10 ml) and cooled to 0° C. 3-(hexadecyloxy)propyl phosphorodichloridate, R (0.456 g, 1.1 mmol) was added portionwise. The reaction mixture was allowed to warm to room temperature and was stirred for 40 h. The solvent was evaporated under reduced pressure and the residue was purified by silica gel column chromatography eluting with dichloromethane/methanol (6:4) to give 42 mg of ((2R,3S,4S,5R)-5-(4-amino-5-carbamoyl-7H-pyrrolo[2,3-d]pyrimidin-7-yl)-4-azido-3-hydroxytetrahydrofuran-2-yl)methyl 3-(hexadecyloxy)propyl hydrogen phosphate, 36 as an off white solid. LC/MS: M⁺ 697.4 (C₃₁H₅₃N₈O₈P, M.W: 696.37); ¹H NMR (300 MHz, DMSO): 8.60-8.62 (d, 1H), 8.22 (s, 1H), 8.06 (s, 1H), 7.51-7.53 (d, 1H), 6.52-6.56 (d, 1H), 4.63-4.68 (m, 1H), 4.25-4.30 (m, 2H), 3.94-4.08 (m, 2H), 3.83-3.90 (m, 2H), 3.21-3.40 (m, 4H), 1.75-1.79 (m, 2H), 1.41-1.43 (m, 2H), 1.21 (s, 26H), 0.82-0.86 (m, 3H). ³¹P NMR (300 MHz, DMSO): Single peak

### Example 27 and 28

### Step 1: Preparation of [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-(3-(hexadecyloxy)propyl)cyclic phosphate, 37 and [4-amino-7-((2R,3R,4S,5R)-3,4-dihydroxy-5-(hydroxymethyl)tetrahydrofuran-2-yl)-7H-pyrrolo[2,3-d]pyrimidine-5-carbothioamide-3′,5′-(3-(hexadecyloxy)propyl)cyclic phosphate, 38.

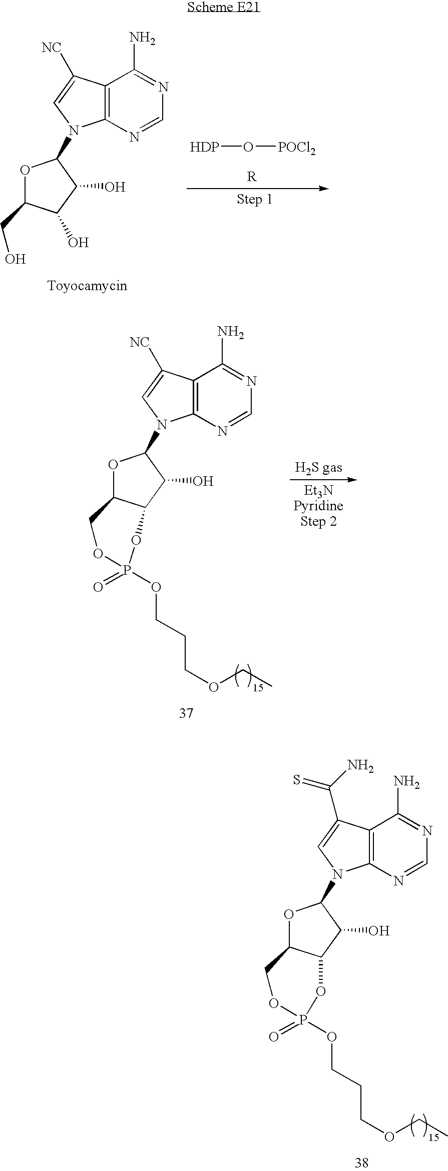

### Step 1: Preparation of [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-(3-(hexadecyloxy)propyl)cyclic phosphate, 37

Toyocamycin (291 mg, 1 mmol) was placed in a 40 mL vial, dioxane (15 mL) was added to make a suspension and the mixture was placed in a room temperature water bath. Sodium bis(trimethylsilyl)amide (2 mL, 2.0 mmol) was added. The mixture was stirred at room temperature for 30 minutes, then a solution of 3-(hexadecyloxy)propyl phosphoro dichloridate, R (417 mg, 1 mmol in dioxane (5 mL) was added. The mixture was stirred at room temperature for 3 hours. LC-MS indicated only small portion of desired product (M+1 636) and a second component with (M+1 654). Heated the mixture to 60° C. overnight. Cooled the reaction mixture to room temperature and quenched with saturated aqueous ammonium chloride solution. The mixture was extracted with dichloromethane (a solid was removed via filtration that was mostly the acyclic product). The dichloromethane phase was applied to a silica gel column and eluted using a gradient of dichloromethante to 10% methanol/dichloromethane to give 58 mg of 37. NMR analysis indicated the presence of some HDP-OH contamination. This material was used without further purification

### Step 2: Preparation of [4-amino-7-((2R,3R,4S,5R)-3,4-dihydroxy-5-(hydroxymethyl)tetrahydrofuran-2-yl)-7H-pyrrolo[2,3-d]pyrimidine-5-carbothioamide-3′,5′-(3-(hexadecyloxy)propyl)cyclic phosphate, 38

[4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-(3-(hexadecyloxy)propyl)cyclic phosphate, 37 (0.022 g, 0.034 mmol) was dissolved in anhydrous pyridine (5 ml) under an inert atmosphere and a catalytic amount of Et₃N (5 mol %) was added. A slow stream of hydrogen sulfide gas (Stainless steel lecture cylinder with T-purge valve, Cat log No: 295442-227G from Aldrich) was passed through the reaction mixture for 3 h. After completion of the reaction, solvent was evaporated under reduced pressure and the residue was purified by silica gel column chromatography eluting with dichloromethane:methanol (8:2) to give 2 mg (9%) of 38. LC/MS: M⁺ 670.2 (C₃₁H₅₂N₅O₇PS, M.W: 669.33); ¹H NMR (300 MHz, DMSO): 8.17 (s, 1H), 7.61 (s, 1H), 7.10-7.19 (m, 1H), 5.94-5.95 (m, 1H), 5.19-5.20 (m, 1H), 4.59-4.66 (m, 2H), 4.33-4.35 (m, 2H), 4.27-4.29 (m, 2H), 3.50-3.55 (m 2H), 3.29-3.42 (m, 2H), 1.97-2.05 (m, 2H), 1.52-1.53 (m, 2H), 1.25 (s, 26H), 0.83-0.85 (m, 3H)

### Example 29

### Preparation of 4-amino-7-((2R,3S,4S,5R)-3-azido-4-hydroxy-5-(hydroxymethyl) tetrahydrofuran-2-yl)-7H-pyrrolo[2,3-d]pyrimidine-5-carboximidamide, 42

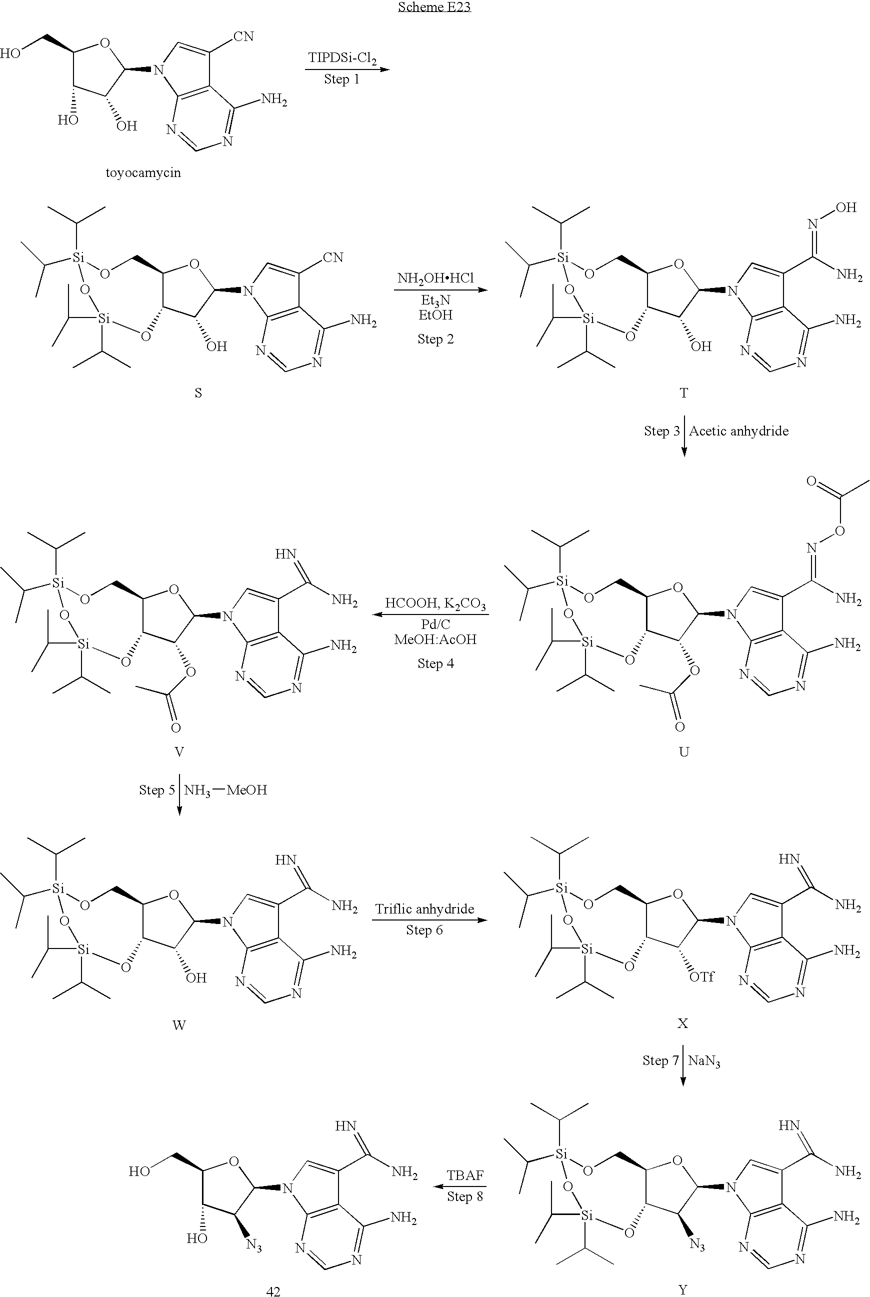

### Step 1: Preparation of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbonitrile, S

Toyocamycin, (1.46 g, 5 mmol) was taken up in 15 ml of anhydrous pyridine and cooled to 0° C. After 10 minutes, 1,3-dichloro-1,1,3,3-tetraisopropyldisiloxane, TIPDSi-Cl₂ (1.73 g, 5.5 mmol) was added and the reaction mixture was allowed to warm to room temperature. The reaction mixture was stirred for 3 hr, quenched with saturated aqueous NaHCO₃ solution (5 ml) and extracted with dichloromethane (3×25 ml). The organic phase was washed with water, brine and dried over sodium sulfate. The solvent was evaporated under reduced pressure and the residue was purified by silica gel column chromatography eluting with Hexane:EtOAc (1:1) to give 1.3 g (Yield 50%) of compound S. This material was used as is for the next reaction.

### Step 2: Preparation of (Z)-4-amino-N′-hydroxy-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, T

4-Amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbonitrile, S (1.2 g, 2.2 mmol) was dissolved in anhydrous ethanol (15 mL) and triethylamine (0.68 g, 6.6 mol) was added, followed by hydroxylamine hydrochloride (0.39 g, 5.6 mmol). The reaction mixture was transferred to a pre-heated oil bath and stirred at 80° C. for 24 h. After completion of the reaction, solvent was removed under reduced pressure and residue was washed with Hexane/EtOAc (1:1) to give 0.61 g (Yield 50%) of (Z)-4-amino-N′-hydroxy-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, T. This material was used as is for next reaction.

### Step 3: Preparation of (6aR,8R,9R,9aR)-8-(5-((Z)-N′-acetoxycarbamimidoyl)-4-amino-7H-pyrrolo[2,3-d]pyrimidin-7-yl)-2,2,4,4-tetraisopropyltetrahydro-6H-furo[3,2-f][1,3,5,2,4]trioxadisilocin-9-yl acetate, U

(Z)-4-Amino-N′-hydroxy-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, T (0.6 g, 1.0 mmol) was dissolved in a mixture of dichloromethane and pyridine/7:3 (10 ml) and the mixture was cooled to 0° C. After 10 minutes acetic anhydride (1 ml) was added, the mixture was warmed to room temperature and stirred for 4 h. After completion of the reaction, solvent was removed under reduced pressure and residue was purified by silica gel column chromatography eluting with dichloromethane:methanol (9:1) to give 0.47 g (Yield 69%) of (6aR,8R,9R,9aR)-8-(5-((Z)-N′-acetoxycarbamimidoyl)-4-amino-7H-pyrrolo[2,3-d]pyrimidin-7-yl)-2,2,4,4-tetraisopropyltetrahydro-6H-furo[3,2-f][1,3,5,2,4]trioxadisilocin-9-yl acetate, U. This material was used without further characterization.

### Step 4: Preparation of (6aR,8R,9R,9aR)-8-(4-amino-5-carbamimidoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl acetate, V

(6aR,8R,9R,9aR)-8-(5-((Z)-N′-Acetoxycarbamimidoyl)-4-amino-7H-pyrrolo[2,3-d]pyrimidin-7-yl)-2,2,4,4-tetraisopropyltetrahydro-6H-furo[3,2-f][1,3,5,2,4]trioxadisilocin-9-yl acetate, U (450 mg, 0.69 mmol) was dissolved in methanol/acetic acid (3:2) (10 ml) under an inert atmosphere and formic acid (0.32 ml, 6.9 mmol), potassium carbonate (0.48 g, 3.4 mmol) and activated Pd/C (10 mole %) was added. The reaction mixture was stirred at room temperature for 12 h. After completion of the reaction as indicated by LC/MS, the mixture was filtered through celite and washed with methanol (3×20 ml). The combined solvents were removed under reduced pressure and residue was purified by silica gel column chromatography eluting with dichloromethane/methanol (8:2) to give 0.29 g (Yield 59%) of (6aR,8R,9R,9aR)-8-(4-amino-5-carbamimidoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl acetate isolated, V. This material was used without further characterization.

### Step 5: Preparation of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, W

(6aR,8R,9R,9aR)-8-(4-Amino-5-carbamimidoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl acetate, V (290 mg, 0.49 mmol) was dissolved in 1.0 M ammonia in methanol (5 ml), placed in a sealed tube and stirred for 24 h at room temperature. After completion of the reaction, the solvent was removed under reduced pressure. The residue was purified by silica gel column chromatography eluting with dichloromethane/methanol (7:3) to give 152 mg (Yield 56%) of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, W. This material was used without further characterization.

### Step 6: Preparation of (6 aR,8R,9R,9aR)-8-(4-amino-5-carbamimidoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethane sulfonate, X

4-Amino-7-((6aR,8R,9R,9aS)-9-hydroxy-2,2,4,4-tetraisopropyl tetrahydro-6H-furo[3,2-f][1,3,5,2,4]trioxadisilocin-8-yl)-7H-pyrrolo[2,3-d]pyrimidine-5-carboximidamide, W (150 mg, 0.27 mmol) was taken up in anhydrous pyridine (10 ml) and the mixture was cooled to −10° C. After 10 minutes, trifluoromethanesulfonic anhydride (0.08 ml, 0.29 mmol) was added dropwise via a syringe. The reaction mixture was warmed to 0° C. and stirred for 4 h. After completion of the reaction, the reaction mixture was quenched with saturated aqueous NaHCO₃ solution (5 ml) and extracted with dichloromethane (3×25 ml). The organic phase was washed with water, brine and dried over sodium sulfate. The solvent was evaporated under reduced pressure and the residue was purified by silica gel column chromatography eluting with dichloromethane/methanol (7:3) to give 99 mg (Yield 54%) of (6aR,8R,9R,9aR)-8-(4-amino-5-carbamimidoyl-7H-pyrrolo[2,3-d]pyrimidin-7-yl)-2,2,4,4-tetraisopropyl tetrahydro-6H-furo[3,2-f][1,3,5,2,4]trioxadisilocin-9-yl trifluoromethanesulfonate, X. This material was used without further characterization.

### Step 7: Preparation of 4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, Y

(6aR,8R,9R,9aR)-8-(4-Amino-5-carbamimidoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethanesulfonate, X (90 mg, 0.13 mmol) was taken up in anhydrous dimethylformamide (10 ml) under an inert atmosphere and sodium azide (0.35 mg, 0.52 mmol) was added. The reaction mixture was heated to 60-80° C. for 12 h. After completion of the reaction, the reaction mixture was quenched with water (10 ml) and extracted with ethyl acetate (3×20 ml). The organic phase was washed with water, brine and dried over sodium sulfate. The solvent was evaporated under reduced pressure and the residue was purified by silica gel column chromatography eluting with dichloromethane/methanol (8:2) to give 41 mg (Yield 54%) of 4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide, Y. This material was used without further characterization.

### Step 8: Preparation of 4-amino-7-((2R,3S,4S,5R)-3-azido-4-hydroxy-5-(hydroxymethyl) tetrahydrofuran-2-yl)-7H-pyrrolo[2,3-d]pyrimidine-5-carboximidamide, 42

4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboximidamide (40 mg, 0.06 mmol), Y was dissolved in anhydrous THF (2 ml) pre-cooled to 0° C. and allowed to stir at 0° C. for 10 minutes. At that time 1.0 M TBAF in THF (0.2 ml) was added and the mixture was allowed to warm to room temperature and stir for 1-2 h. After completion of the reaction, the solvent was evaporated under reduced pressure and the residue was purified by silica column chromatography eluting with dichloromethane/methanol (7:3) to give 11 mg (Yield 47%) of 4-amino-7-((2R,3S,4S,5R)-3-azido-4-hydroxy-5-(hydroxymethyl) tetrahydrofuran-2-yl)-7H-pyrrolo[2,3-d]pyrimidine-5-carboximidamide, 42; LC/MS: M⁺334.16 (C₁₂H₁₅N₉O₃, M.W: 333.13); ¹H NMR (300 MHz, CD₃OD): 8.23 (s, 1H), 8.16 (s, 1H), 6.41-6.42 (d, 1H), 5.82-5.85 (m, 1H), 5.49-5.55 (m, 1H), 4.47-4.48 (m, 1H), 3.75-3.78 (m, 2H).

### Examples 30 and 31

### Preparation of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide, 43 and 4-amino-7-((2R,3S,4S,5R)-3-azido-4-hydroxy-5-(hydroxymethyl)tetrahydrofuran-2-yl)-7H-pyrrolo[2,3-d]pyrimidine-5-carboxamide, 35.

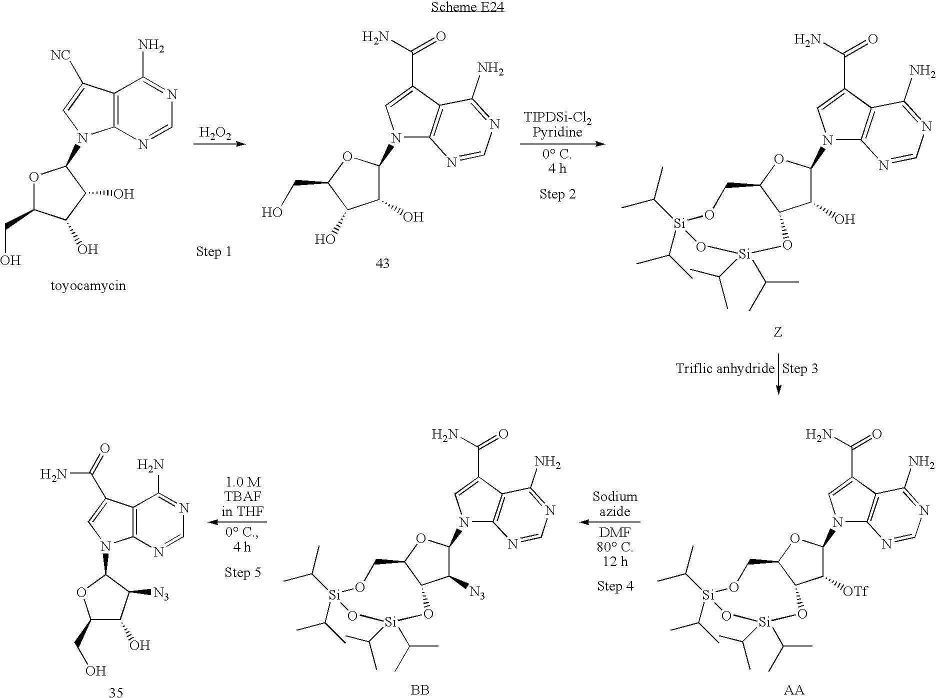

### Step 1: Preparation of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide, 43

Toyocamycin, (2.9 g, 1 mmol) was dissolved in saturated aqueous K₂CO₃ solution (50 ml) and cooled to 0° C. After 15 minutes H₂O₂(10 ml) was added. The mixture was allowed to warm to room temperature and stirred for 12 h. After completion of the reaction as indicated by LC/MS, the solid was filtered, washed with water and dried under vacuum for 12 h and to give 2.1 g (Yield 68%) of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide, 43.

### Step 2: Preparation of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, Z

4-Amino-7-((2R,3R,4S,5R)-3,4-dihydroxy-5-(hydroxymethyl)tetrahydrofuran-2-yl)-7H-pyrrolo[2,3-d]pyrimidine-5-carboxamide, 43 (2.1 g, 6.7 mmol) was dissolved in anhydrous pyridine (15 ml) and cooled to 0° C., After 10 minutes, TIPDSi-Cl₂ (2.5 g, 8.1 mmol) was added and the mixture was allowed to warm to room temperature. After 3 hr, the reaction mixture was quenched with saturated aqueous NaHCO₃ solution (5 ml) and extracted with dichloromethane (3×50 ml). The organic phase was washed with water, brine and dried over sodium sulfate. The solvent was evaporated under reduced pressure and the residue was purified by silica gel column chromatography eluting with hexanes/ethyl acetate (1:1) to give 2.3 g (Yield 62%) of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, Z.

### Step 3: Preparation of (6aR,8R,9R,9aR)-8-(4-amino-5-carbamoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethanesulfonate, AA

4-Amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, Z (2.3 g, 4.1 mmol) was taken up in anhydrous pyridine (25 ml) and the mixture was cooled to −10° C. After 14 minutes, trifluoromethanesulfonic anhydride (1.47 g, 5.2 mmol) was added dropwise via a syringe. The reaction mixture was allowed to warm to 0° C. After 4 h at 0° C., the reaction mixture was quenched with saturated aqueous NaHCO₃ solution (10 ml) and extracted with dichloromethane (3×50 ml). The organic phase was washed with water, brine and dried over sodium sulfate. The solvent was evaporated under reduced pressure and the residue was purified by silica gel column chromatography eluting with hexanes/ethyl acetate (7:3) to give 2.0 g (Yield 70%) of (6aR,8R,9R,9aR)-8-(4-amino-5-carbamoyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethanesulfonate, AA.

### Step 4: Preparation of 4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, BB

(6aR,8R,9R,9aR)-8-(4-Amino-5-carbamoyl-7H-pyrrolo[2,3-d]pyrimidin-7-yl)-2,2,4,4-tetra isopropyl tetrahydro-6H-furo[3,2-f][1,3,5,2,4]trioxadisilocin-9-yl trifluoromethanesulfonate, AA (2.0 g, 2.9 mmol) was taken up in anhydrous dimethylformamide (20 ml) under an inert atmosphere and sodium azide (0.76 g, 11.6 mmol) was added. The reaction mixture was heated at 60-80° C. for 12 h. The reaction mixture was quenched with water (15 ml) and extracted with ethyl acetate (3×50 ml). The organic phase was washed with water, brine and dried over sodium sulfate. The solvent was evaporated under reduced pressure and the residue was purified by silica gel column chromatography eluting with hexanes/ethyl acetate (7:3) to give 1.1 g (Yield 68%) of 4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, BB.

### Step 5: Preparation of 4-amino-7-((2R,3S,4S,5R)-3-azido-4-hydroxy-5-(hydroxymethyl)tetrahydrofuran-2-yl)-7H-pyrrolo[2,3-d]pyrimidine-5-carboxamide, 35

4-Amino-7-((6aR,8R,9R,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, BB (1.1 g, 1.9 mmol) was dissolved in anhydrous THF (15 ml) pre-cooled to 0° C. and allowed to stir at 0° C. After 10 min., 1.0 M TBAF in THF (2.5 ml) was added and the mixture was warmed to room temperature and stirred for 3-4 h. After completion of the reaction, the solvent was evaporated under reduced pressure and the residue was purified by silica column chromatography eluting with dichloromethane/methanol (7:3) to give 340 mg (Yield 53%) of 4-amino-7-((2R,3S,4S,5R)-3-azido-4-hydroxy-5-(hydroxymethyl)tetrahydrofuran-2-yl)-7H-pyrrolo[2,3-d]pyrimidine-5-carboxamide, 35. This material was used without further characterization for the synthesis of compound 36.

### Example 32

### Alternative Synthesis of Preparation of 4-amino-7-((2R,3S,4S,5R)-3-azido-4-hydroxy-5-(hydroxymethyl)tetrahydrofuran-2-yl)-7H-pyrrolo[2,3-d]pyrimidine-5-carboxamide, 35

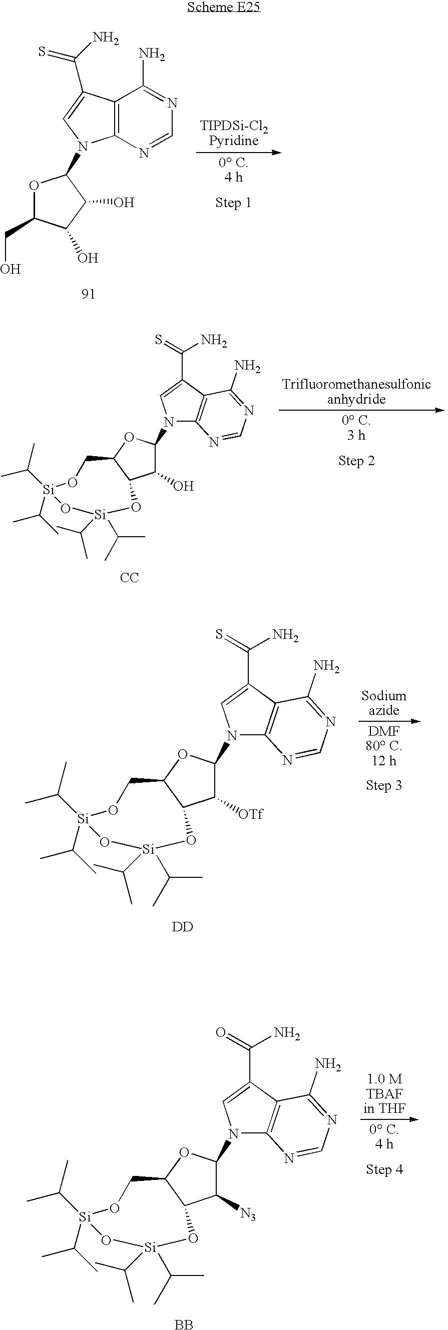

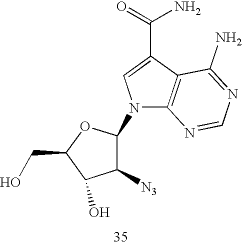

### Step 1: Preparation of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbothioamide, CC

4-Amino-7-((2R,3R,4S,5R)-3,4-dihydroxy-5-(hydroxymethyl)tetrahydrofuran-2-yl)-7H-pyrrolo[2,3-d]pyrimidine-5-carbothioamide, 91 (2.1 g, 6.4 mmol) was placed in anhydrous pyridine (20 mL) and cooled to 0° C. After 10 minutes, TIPDSi-Cl₂ (2.23 g, 7.0 mmol) was added and the reaction mixture was allowed to warm to room temperature. After stirring for 4-5 h, the reaction was stopped and quenched with saturated aqueous NaHCO₃ solution (15 ml). The mixture was extracted with dichloromethane (3×50 ml) and the organic phase was washed with water, brine and dried over sodium sulfate. The solvent was evaporated under reduced pressure and the residue was purified by silica gel column chromatography eluting with dichloromethane:Methanol (9:1) to give 1.1 g (30%) of 4-amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbothioamide, CC.

### Step 2: Preparation of (6aR,8R,9R,9aR)-8-(4-amino-5-carbamothioyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethanesulfonate, DD

4-Amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbothioamide, CC (1.1 g, 1.9 mmol) was taken up anhydrous pyridine (20 mL) and cooled to −10° C. After 14 minutes, trifluoromethanesulfonic anhydride (0.6 g, 2.1 mmol) was added dropwise via syringe. The reaction mixture was warmed to 0° C. and stirred for 3 h. After completion of the reaction, reaction mixture was quenched with saturated aqueous NaHCO₃ solution (10 ml) and extracted with dichloromethane (3×50 ml). The organic phase was washed with water, brine and dried over sodium sulfate. The solvent was evaporated under reduced pressure and the residue was purified by silica gel column chromatography eluting with hexane/EtOAc (1:1) to give 0.7 g (51%) of (6aR,8R,9R,9aR)-8-(4-amino-5-carbamothioyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethanesulfonate, DD.

### Step 3: Preparation of 4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, BB

(6aR,8R,9R,9aR)-8-(4-Amino-5-carbamothioyl-7H-pyrrolo[2,3-d]pyrimidin-7-yl)-2,2,4,4-tetraisopropyltetrahydro-6H-furo[3,2-f][1,3,5,2,4]trioxadisilocin-9-yl trifluoromethanesulfonate, DD (0.7 g, 1.0 mmol) was placed in anhydrous DMF (20 mL) under an inert atmosphere and sodium azide (0.26 g, 4.0 mmol) was added. The reaction mixture was heated up to 60-80° C. for 12 h. LC/MS was indicated only 577 M⁺ not the expected M⁺ 593. The reaction was stopped and quenched with water (15 ml) and extracted with ethyl acetate (3×25 ml). The organic phase was washed with water, brine and dried over sodium sulfate. The solvent was evaporated under reduced pressure and the residue was purified by silica column chromatography eluting with hexane/EtOAc (1:1) to give 0.26 g (45%) of 4-amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, BB.

### Step 4: Preparation of 4-amino-7-((2R,3S,4S,5R)-3-azido-4-hydroxy-5-(hydroxymethyl) tetrahydrofuran-2-yl)-7H-pyrrolo[2,3-d]pyrimidine-5-carboxamide, 35

4-Amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carboxamide, BB (0.22 g, 0.38 mmol) was dissolved in pre-cooled anhydrous THF (10 ml) and allowed to stir at 0° C. for 10 minutes. At that time, 1.0 M TBAF in THF (2.0 ml) of was added and the mixture was warmed to room temperature and stirred for 3-4 h. After completion of the reaction, the solvent was evaporated under reduced pressure and the residue was purified by silica gel column chromatography eluting with dichloromethane/methanol (6:4) to give 51 mg (40%) of 4-amino-7-((2R,3S,4S,5R)-3-azido-4-hydroxy-5-(hydroxymethyl)tetrahydrofuran-2-yl)-7H-pyrrolo[2,3-d]pyrimidine-5-carboxamide, 35. LC/MS: M⁺335.1 (C₁₂H₁₄N₈O₄, M.W: 334.11). ¹H NMR (300 MHz, CD₃OD): 8.50 (s, 1H), 8.40 (s, 1H), 6.34-6.36 (d, 1H), 4.49-4.51 (m, 1H), 4.29-4.31 (m, 1H), 4.16-4.17 (m, 1H), 3.84-3.88 (m, 2H).

### Examples 33 and 34

### Preparation of 4-amino-7-((2R,3S,4S,5R)-3-azido-4-hydroxy-5-(hydroxymethyl)tetrahydrofuran-2-yl)-7H-pyrrolo[2,3-d]pyrimidine-5-carbonitrile, 44 and 4-amino-7-((2R,3S,4S,5R)-3-azido-4-hydroxy-5-(hydroxymethyl)tetrahydrofuran-2-yl)-7H-pyrrolo[2,3-d]pyrimidine-5-carbothioamide, 45.

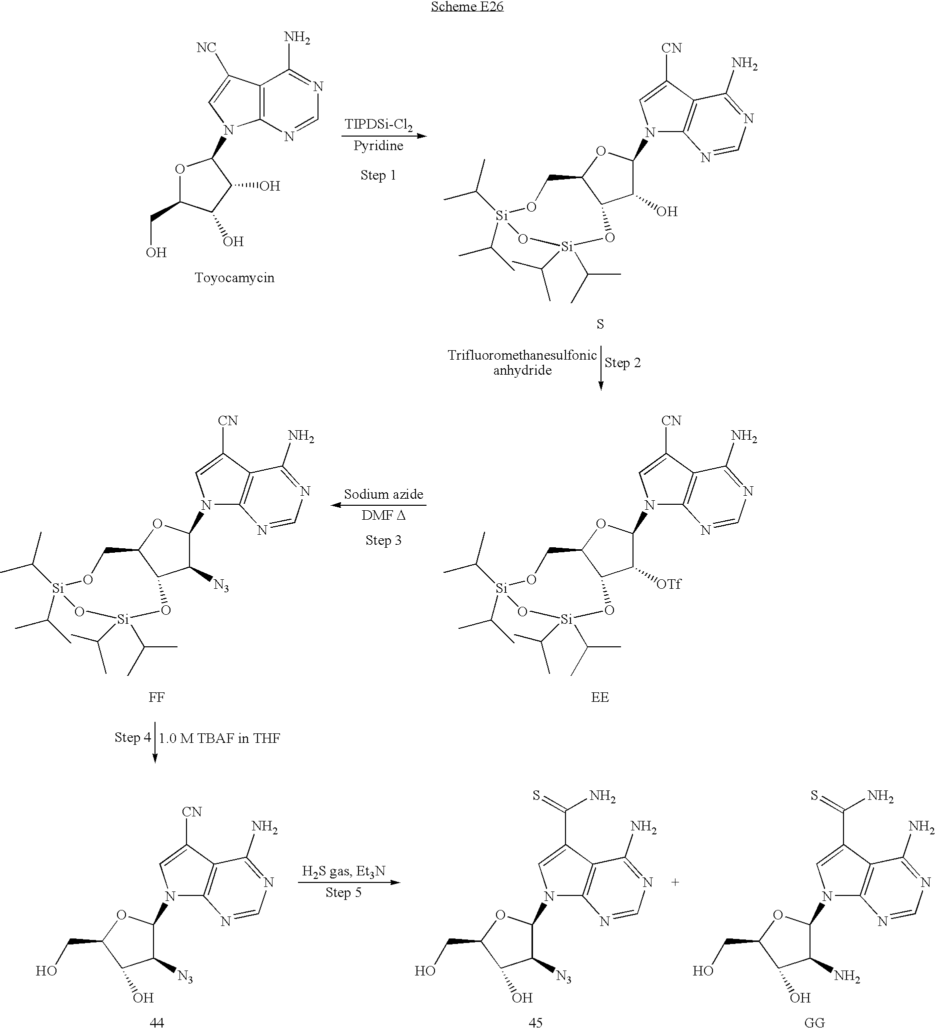

### Step 1: 4-Amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbonitrile, S

Toyocamycin (1.46 g, 5 mmol) was dissolved in anhydrous pyridine (15 mL) and cooled to 0° C. TIPDSi-Cl₂ (1.73 g, 5.5 mmol) was added and the reaction was allowed to warm to room temperature. It was stirred for 3 hours. The reaction mixture was then quenched with saturated NaHCO₃ solution (5 ml) and extracted with dichloromethane (3×25 ml). The combined dichloromethane extracts were washed with water and brine. The dichloromethane layer was dried over sodium sulfate, filtered and concentrated in vacuo. The residue was purified by column chromatography (silica gel). The eluting solvent was hexane:ethyl acetate (1:1). The isolated compound, S was taken forward to the next reaction. 1.3 g (50%) obtained.

### Step 2: 4-Amino-7-((6aR,8R,9R,9aS)-9-hydroxy-2,2,4,4-tetraisopropyltetrahydro-6H-furo[3,2-f][1,3,5,2,4]trioxadisilocin-8-yl)-7H-pyrrolo[2,3-d]pyrimidine-5-carbonitrile, EE

Compound S (1.2 g, 2.2 mmol) was dissolved in anhydrous pyridine (20 mL) and cooled to −10° C. After 14 minutes, trifluoromethanesulfonic anhydride (0.7 g, 2.4 mmol) was added drop wise through a syringe. The reaction mixture was warmed to 0° C. and stirred for 4 hours. The reaction mixture was then quenched with saturated NaHCO₃ solution (5 ml) and extracted with dichloromethane (3×25 ml). The combined dichloromethane extracts were washed with water then brine. The dichloromethane layer was separated and dried over sodium sulfate. The solvent was evaporated under reduced pressure and the residue was purified by column chromatography (silica gel). The eluting solvent was hexanes:ethyl acetate (7:3). The isolated compound, EE was taken forward to the next reaction. 0.9 g (60%) obtained.

### Step 3: 4-Amino-7-((6aR,8R,9S,9aS)-9-azido-2,2,4,4-tetraisopropyltetrahydro-6H-furo[3,2-f][1,3,5,2,4]trioxadisilocin-8-yl)-7H-pyrrolo[2,3-d]pyrimidine-5-carbonitrile, FF

Compound EE (0.9 g, 1.3 mmol) was dissolved in anhydrous dimethylformamide (20 mL) and to this solution was added sodium azide (0.35 g, 5.4 mmol). The reaction mixture was heated to 60-80° C. and stirred for 12 hours. The reaction mixture was quenched with water (15 ml) and extracted with ethyl acetate (3×25 ml). The combined ethyl acetate extracts were washed with water then brine. The ethyl acetate was separated and dried over sodium sulfate. The solvent was evaporated under reduced pressure and the residue was purified by column chromatography (silica gel). The eluting solvent was hexanes:ethyl acetate (7:3). The isolated compound, FF was taken forward to the next reaction. 0.36 g (48%) obtained.

### Step 4: 4-Amino-7-((2R,3S,4S,5R)-3-azido-4-hydroxy-5-(hydroxymethyl)tetrahydrofuran-2-yl)-7H-pyrrolo[2,3-d]pyrimidine-5-carbonitrile, 44

Compound FF (0.3 g, 0.53 mmol) was dissolved in pre-cooled anhydrous THF (15 ml) and allowed to stir at 0° C. for 10 minutes. At that time, tetrabutylammonium fluoride in THF (1.0 M, 2.5 mL) was added and the reaction was warmed to room temperature. The reaction stirred for 3-4 h. The solvent was evaporated under reduced pressure and the residue was purified by column chromatography (silica gel). The eluting solvent was dichloromethane:methanol (7:3). The isolated compound, 4-amino-7-((2R,3S,4S,5R)-3-azido-4-hydroxy-5-(hydroxymethyl)tetrahydrofuran-2-yl)-7H-pyrrolo[2,3-d]pyrimidine-5-carbonitrile, 44 was taken forward to the next reaction. 80 mg (47%) obtained.

### Step 5: 4-Amino-7-((2R,3S,4S,5R)-3-azido-4-hydroxy-5-(hydroxymethyl)tetrahydrofuran-2-yl)-7H-pyrrolo[2,3-d]pyrimidine-5-carbothioamide, 45

Compound 44 (55 mg, 0.17 mmol) was dissolved in 2 ml of anhydrous pyridine. A slow stream of hydrogen sulfide gas (Stainless steel lecture cylinder with T-purge valve, Cat log No: 295442-227G from Aldrich) was passed through the reaction mixture for 8-10 hours. After 8 hours of reaction time, LC/MS indicated the formation of 45 along with the reduced product, GG. Starting compound 44 was also present in approximately 20-30%. The reaction was stopped and the solvent was evaporated under reduced pressure. The residue was purified by column chromatography (silica gel). The eluting solvent was dichloromethane:methanol (8:2). 45: LC/MS: M⁺ 351.0 (C₁₂H₁₄N₈O₃S, M.W: 350.36). ¹H NMR (300 MHz, CD₃OD): 8.12 (s, 1H), 7.96 (s, 1H), 6.60-6.62 (d, 1H), 4.44-4.85 (m, 1H), 3.90-3.91 (m, 1H), 3.84-3.89 (m, 2H), 3.21-3.25 (m, 1H).

### Examples 35 and 36

### Preparation of 4-Amino-7-((3aR,4R,6R,6aR)-6-(hydroxymethyl)-2-oxotetrahydrofuro[3,4-d][1,3]dioxol-4-yl)-7H-pyrrolo[2,3-d]pyrimidine-5-carbonitrile, 46. and 4-amino-7-((3aR,4R,6R,6aR)-6-(hydroxymethyl)-2-oxotetrahydrofuro[3,4-d][1,3]dioxol-4-yl)-7H-pyrrolo[2,3-d]pyrimidine-5-carbothioamide, 47.

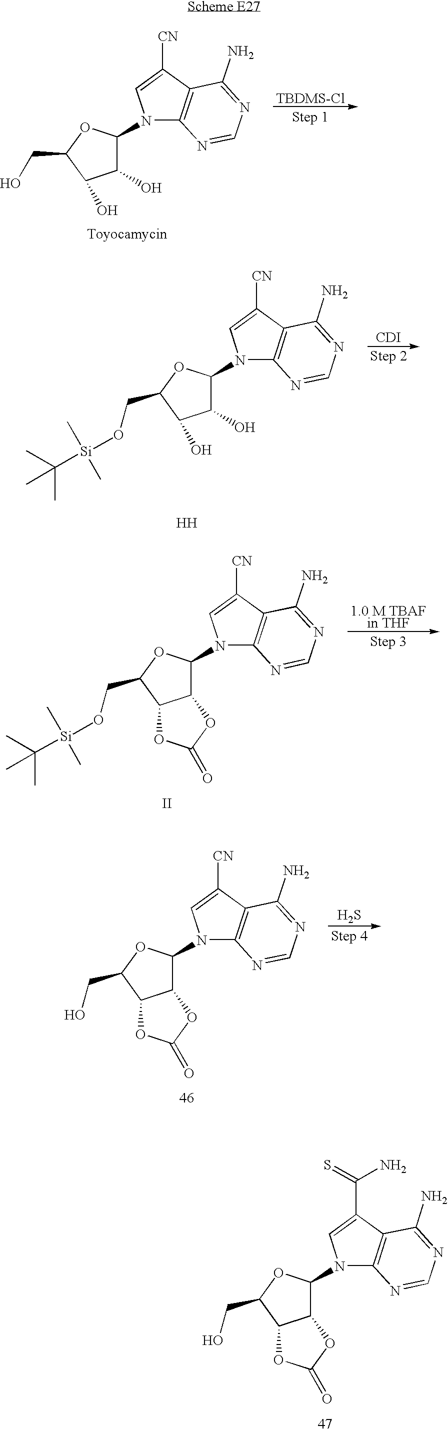

### Step 1: 4-Amino-7-((2R,3R,4S,5R)-5-((tert-butyldimethylsilyloxy)methyl)-3,4-dihydroxytetrahydrofuran-2-yl)-7H-pyrrolo[2,3-d]pyrimidine-5-carbonitrile, HH

Toyocamycin (1.46 g, 5 mmol) was dissolved in anhydrous dimethylformamide (15 mL) and to it was added imidazole (0.68 g, 10 mmol). This mixture was cooled to 0° C. and after 10 minutes, TBDMS-Cl (0.82 g, 5.5 mmol) was added. This was allowed to warm to room temperature and stirred for 3 h. LC/MS indicated the reaction was complete. It also showed 3-5% of di-O-silyl compound. The reaction mixture was quenched with saturated NaHCO₃ solution (5 ml) and extracted with ethyl acetate (3×25 ml). The combined ethyl acetate extracts were washed with water then brine. The ethyl acetate layer was separated and dried over sodium sulfate. The solvent was evaporated under reduced pressure and the residue was purified by column chromatography (silica gel). The eluting solvent was DCM:Methanol (9:1). The isolated compound was taken forward to the next reaction. 1.2 g (60%) obtained.

### Step 2: 4-Amino-7-((3aR,4R,6R,6aR)-6-((tert-butyldimethylsilyloxy)methyl)-2-oxotetrahydrofuro[3,4-d][1,3]dioxol-4-yl)-7H-pyrrolo[2,3-d]pyrimidine-5-carbonitrile, II

Compound HH (1.0 g, 2.5 mmol) was dissolved in anhydrous dimethylformamide (10 mL) and cooled to 0° C. After 15 minutes at 0° C., carbonyldiimmidazole (CDI) (0.9 g, 5.5 mmol) was added. The reaction mixture was warmed to room temperature and stirred for 24 hours. When the reaction was completed, it was quenched with water (10 ml) and extracted with ethyl acetate (3×25 ml). The combined ethyl acetate extracts were washed with water then brine. The ethyl acetate layer was separated and dried over sodium sulfate. The solvent was evaporated under reduced pressure and the residue was purified by column chromatography. The eluting solvent was dichloromethane:methanol (8:2). The isolated 2,3-carbonate compound II was taken forward to the next reaction. 0.55 g (52%) obtained.

### Step 3: 4-Amino-7-((3aR,4R,6R,6aR)-6-(hydroxymethyl)-2-oxotetrahydrofuro[3,4-d][1,3]dioxol-4-yl)-7H-pyrrolo[2,3-d]pyrimidine-5-carbonitrile, 46

Compound II (0.5 g, 1.16 mmol) was dissolved in pre-cooled anhydrous THF (5 ml) and allowed to stir at 0° C. for 10 minutes. At that time, tetrabutylammonium fluoride in tetrahydrofuran (0.5 ml, 1.0 M) was added and the reaction was warmed to room temperature. It was stirred for 1-2 hours. The solvent was evaporated under reduced pressure and the residue was purified by column chromatography. The eluting solvent was dichloromethane:methanol

(7:3). The isolated 4-amino-7-((3aR,4R,6R,6aR)-6-(hydroxymethyl)-2-oxotetrahydrofuro[3,4-d][1,3]dioxol-4-yl)-7H-pyrrolo[2,3-d]pyrimidine-5-carbonitrile, 46 was taken forward to the next reaction. 0.163 g (45%) obtained. LC/MS: M⁺ 318.7 (C₁₃H₁₁N₅O₅, M.W: 317.26). ¹H NMR (300 MHz, CD₃OD): 8.23 (s, 1H), 8.16 (s, 1H), 6.41-6.42 (d, 1H), 5.82-5.85 (m, 1H), 5.49-5.55 (m, 1H), 4.47-4.48 (m, 1H), 3.75-3.78 (m, 2H).

### Step 4: 4-amino-7-((3aR,4R,6R,6aR)-6-(hydroxymethyl)-2-oxotetrahydrofuro[3,4-d][1,3]dioxol-4-yl)-7H-pyrrolo[2,3-d]pyrimidine-5-carbothioamide, 47

46 (0.15 g, 0.47 mmol) was dissolved in anhydrous pyridine (5 mL) and to it added a catalytic amount of triethylamine. A slow stream of hydrogen sulfide gas (Stainless steel lecture cylinder with T-purge valve, Cat log No: 295442-227G from Aldrich) was passed through the reaction mixture for 1 hour. The solvent was evaporated under reduced pressure and the residue was purified by column chromatography. The eluting solvent was dichloromethane:methanol (6:4). 47 was isolated in a 30% yield (49 mg). LC/MS: M⁺ 352.8 (C₁₃H₁₃N₅O₅S, M.W: 351.34). ¹H NMR (300 MHz, CD₃OD): 8.12 (s, 1H), 7.82 (s, 1H), 6.36-6.37 (d, 1H), 5.80-5.83 (m, 1H), 5.52-5.55 (m, 1H), 4.31-4.44 (m, 1H), 3.75-3.81 (m, 2H), 3.31-3.34 (m, 1H).

### Examples 37 and 38

### Preparation of (2R,3R)-5-(benzoyloxymethyl)-2-(4-chloro-5-iodo-7H-pyrrolo[2,3-d]pyrimidin-7-yl)-3-methyltetrahydrofuran-3,4-diyl dibenzoate, 56 and (2R,3R)-2-(4-amino-5-iodo-7H-pyrrolo[2,3-d]pyrimidin-7-yl)-5-(hydroxymethyl)-3-methyltetrahydrofuran-3,4-diol, 57

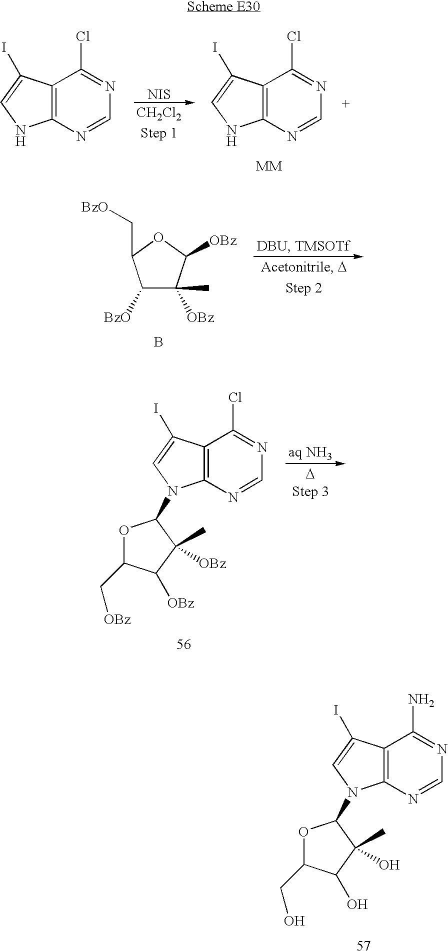

### Step 1: 4-chloro-5-iodo-7H-pyrrolo[2,3-d]pyrimidine, MM

To a solution of 4-chloro-7H-pyrrolo[2,3-d]pyrimidine (8 g, 52.3 mmol) in CH₂Cl₂ (200 ml) was added NIS (14 g, 62.5 ml). The mixture was stirred at rt for 5 h. LC-MS indicated complete reaction. The mixture was filtered and the filtered solid was washed with CH₂Cl₂ (50 ml), followed by hot water (500 ml). The solid was then dried in vacuum oven at 40° C. for 2 days to give 4-chloro-5-iodo-7H-pyrrolo[2,3-d]pyrimidine, MM as a gray solid (13.6 g, 93%). ¹H NMR (DMSO-d₆) δ 12.97 (br s, 1H), 8.60 (s, 1H), 7.95 (s, 1H). LC/MS m/z 279.9 (M+H).

### Step 2: (2R,3R)-5-(benzoyloxymethyl)-2-(4-chloro-5-iodo-7H-pyrrolo[2,3-d]pyrimidin-7-yl)-3-methyltetrahydrofuran-3,4-diyl dibenzoate, 56

To a suspension of MM (4.8 g, 20.0 mmol) and B (11.6 g, 20.0 mmol) in anhydrous acetonitrile (200 ml) was added 1,8-Diazabicyclo[5.4.0]undec-7-ene (DBU) (5.5 ml, 30.0 mmol). The mixture was then treated with TMSOTf (7.2 ml, 40.0 mmol). The mixture was stirred at room temp for 30 min and then heated at 80° C. for 21 h. LC-MS analysis revealed a mixture of starting material MM, and product 56 (˜4:1). The mixture was cooled to room temp. A saturated aqueous NaHCO₃ solution (250 ml) was added to the reaction mixture and extracted with EtOAc (2×200 ml). The organics were dried over Na₂SO₄ and concentrated to give orange residue. The orange residue was dissolved in minimal amount of CH₂Cl₂ and loaded onto a column packed with silica/CH₂Cl₂ and eluted with CH₂Cl₂/EtOAc (98:2→96:4). The product was obtained as a mixture of 56 and sugar impurities (12.2 g, ˜85% pure, 83%). The mixture was used for the next step without further purification.

### Step 3: (2R,3R)-2-(4-amino-5-iodo-7H-pyrrolo[2,3-d]pyrimidin-7-yl)-5-(hydroxymethyl)-3-methyltetrahydrofuran-3,4-diol, 57

A mixture of 56 (6.0 g, 8.1 mmol) and aqueous NH₃ (75 ml) in 1,4-dioxane (75 ml) was stirred at 80° C. for 16 h. LC-MS analysis revealed complete reaction. The mixture was concentrated to dryness. The mixture was then suspended in CH₂Cl₂/MeOH (9:1) and loaded onto a column packed with silica/CH₂Cl₂. The column was eluted with CH₂Cl₂/MeOH (9:1→1:1). The fractions containing required product were collected and concentrated to give 57 as a white solid (2.6 g, 79%).

### Example 39

### Preparation of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carboxylic acid, 58

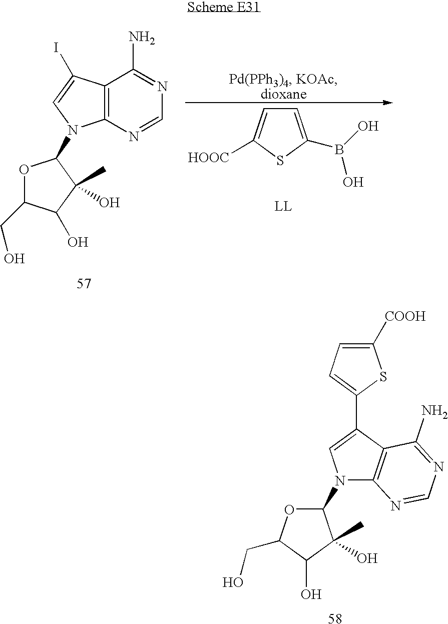

### Preparation of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carboxylic acid, 58

A mixture of (2R,3R)-2-(4-amino-5-iodo-7H-pyrrolo[2,3-d]pyrimidin-7-yl)-5-(hydroxymethyl)-3-methyltetrahydrofuran-3,4-diol, 57 (500 mg, 1.2 mmol), 5-boronothiophene-2-carboxylic acid, LL (311 mg, 1.8 mmol), Pd(PPh₃)₄ (139 mg, 0.12 mmol), and KOAc (353 mg, 3.6 mmol) in 1,4-dioxane (10 ml) was purged with nitrogen for 10 min. The mixture was stirred at 80° C. for 16 h. After cooling to ambient temperature, the mixture was concentrated to dryness. The residue was loaded onto a column packed with silica/CHCl₃. The column was eluted with 10-40% methanol in CHCl₃ containing 0.5% of concentrated aqueous NH₄OH. The fractions containing the product 58 were collected and concentrated to give brown solid (423 mg, 86%). ¹H NMR (CD₃OD): 8.15 (s, 1H), 7.80 (s, 1H), 7.55 (d, 1H, J=3.6 Hz), 7.05 (d, 1H, J=3.6 Hz), 6.30 (s, 1H), 4.1 (m, 3H), 3.8 (m, 1H), 0.88 (m, 3H); LC-MS: 407.1 (M+1).

### Examples 40, 41 and 42

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(3-methylbut-2-enyl)thiophene-2-carboxamide, 59; 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(prop-2-ynyl)thiophene-2-carboxamide, 60; and 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N,N-dimethylthiophene-2-carboxamide, 61

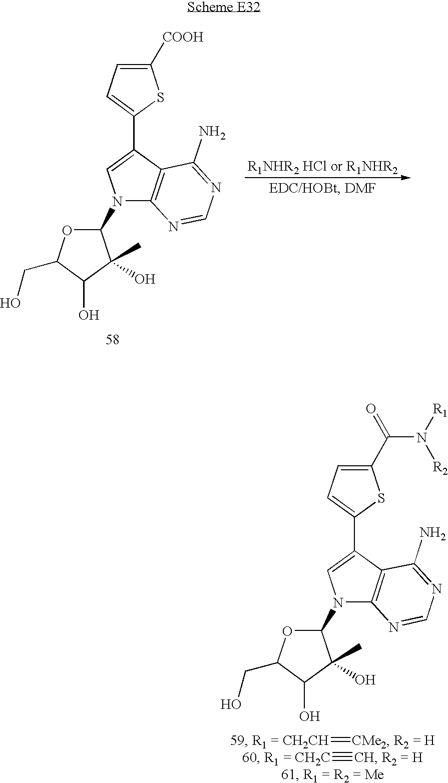

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(3-methylbut-2-enyl)thiophene-2-carboxamide, 59

A mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carboxylic acid, 58 (150 mg, 0.36 mmol), EDC (83 mg, 0.43 mmol), HOBt (64 mg, 0.47 mmol) and DIEA (0.23 mg, 1.5 mmol) in DMF was stirred for 1 h at room temperature. 3-Methylbut-2-en-1-amine hydrochloride (0.1 g, 0.8 mmol) was added to the mixture and the mixture was stirred at room temperature for 16 h. The mixture was concentrated to dryness. The residue was loaded onto a column packed with silica/CHCl₃. The column was eluted with 5-30% methanol in CHCl₃. The product 59 (42 mg) was isolated as a mixture with EDC/HOBt by-product. The mixture was then taken up in 1M aqueous HCl solution and extracted with EtOAc. The EtOAc layer containing the impurity was discarded. The pH of the aqueous layer was adjusted to ˜8 with aqueous K₂CO₃ and extracted with EtOAc. The organics were concentrated to afford 59 as a white solid (5.6 mg, 3%). ¹H NMR (CD₃OD): 8.17 (s, 1H), 7.87 (s, 1H), 7.68 (d, 1H), 7.13 (d, 1H), 6.30 (s, 1H), 5.3 (m, 1H), 4.15 (m, 1H), 4.0 (m, 4H), 3.82 (m, 1H), 1.75 (s, 6H), 0.88 (m, 3H); LC-MS: 474.2 (M+1).

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(prop-2-ynyl)thiophene-2-carboxamide, 60

A mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carboxylic acid, 58 (150 mg, 0.36 mmol), EDC (83 mg, 0.43 mmol), HOBt (64 mg, 0.47 mmol) and DIEA (115 mg, 0.9 mmol) in DMF was stirred for 1 h at room temperature. Propargylamine (0.5 ml) was added to the mixture and the mixture was stirred at room temperature for 16 h. LC-MS analysis indicated ˜10-20% product 60. The mixture was concentrated to dryness. The residue was loaded onto a column packed with silica/CHCl₃. The column was eluted with 5-30% methanol in CHCl₃. The product 60 (42 mg) was isolated as a mixture with EDC/HOBt by-product. The mixture was then taken in 1M aqueous HCl solution and extracted with EtOAc. The EtOAc layer containing the impurity was discarded. The pH of the aqueous layer was adjusted to ˜8 with aqueous K₂CO₃ and extracted with EtOAc. The organics were concentrated to afford 60 as a white solid (7 mg, 5%). ¹H NMR (CD₃OD): 8.17 (s, 1H), 7.90 (m, 1H), 7.71 (d, 1H), 7.15 (d, 1H), 6.30 (s, 1H), 5.3 (m, 1H), 4.14 (m, 3H), 4.10 (m, 2H), 3.86 (m, 1H), 2.62 (s, 1H), 0.88 (m, 3H); LC-MS: 444.2 (M+1).

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N,N-dimethylthiophene-2-carboxamide, 61

A mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carboxylic acid, 58 (150 mg, 0.36 mmol), EDC (83 mg, 0.43 mmol), HOBt (64 mg, 0.47 mmol) and DIEA (115 mg, 0.9 mmol) in DMF was stirred for 1 h at room temp. A solution of dimethylamine in THF (1 ml, 1 mmol, 1.0 M solution) was added to the mixture and the mixture was stirred at room temperature for 16 h. LC-MS analysis indicated ˜10-20% product 61. The mixture was concentrated to dryness. The residue was loaded onto a column packed with silica/CHCl₃. The column was eluted with 5-30% methanol in CHCl₃. The product 61 (65 mg) was isolated as a mixture with EDC/HOBt by-product. The mixture was then taken in 1M aqueous HCl solution and extracted with EtOAc. The EtOAc layer containing the impurity was discarded. The pH of the aqueous layer was adjusted to ˜8 with aqueous K₂CO₃ and extracted with EtOAc. The organics were concentrated to afford 61 as a white solid (17 mg, 11%). ¹H NMR (CD₃OD): 8.17 (s, 1H), 7.87 (s, 1H), 7.51 (d, 1H), 7.13 (d, 1H), 6.30 (s, 1H), 4.17 (m, 1H), 4.05 (m, 2H), 3.83 (m, 1H), 3.3 (m, 6H), 0.88 (m, 3H); LC-MS: 434.2 (M+1).

### Examples 43 and 44

### Preparation of methyl 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylate, 62 and 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylic acid, 63

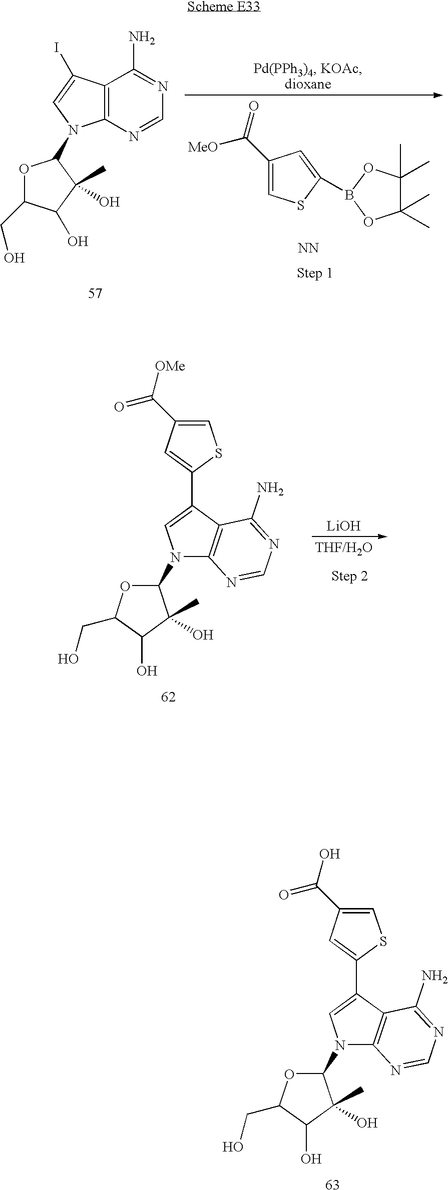

### Step 1: Preparation of methyl 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylate, 62

A mixture of (2R,3R)-2-(4-amino-5-iodo-7H-pyrrolo[2,3-d]pyrimidin-7-yl-5-(hydroxymethyl)-3-methyltetrahydrofuran-3,4-diol, 57 (812 mg, 2.0 mmol), methyl 5-(4,4,5,5-tetramethyl-1,3,2-dioxaborolan-2-yl)thiophene-3-carboxylate, NN (804 mg, 3.0 mmol), Pd(PPh₃)₄ (115 mg, 0.1 mmol), and KOAc (590 mg, 6.0 mmol) in 1,4-dioxane (10 ml) and water (2 ml) was purged with nitrogen for 10 min. The mixture was stirred at 80° C. for 3 h. The mixture was cooled to ambient temperature and concentrated to dryness. The residue was combined with the crude residue from an identical procedure and taken up in a mixture of 1M aqueous HCl (100 ml) and EtOAc (100 ml) and stirred for 5 min. The layers were separated, and the EtOAc layer was discarded. The pH of the aqueous layer was adjusted to ˜8 with aqueous NaHCO₃ solution and extracted with EtOAc (2×50 ml). The organics were dried over Na₂SO₄ and concentrated to give 62 as an off-white solid (794 mg, 60%). ¹H NMR confirmed the product and indicated a single unidentified peak (could be pinacolatoboran by-product). This material was used for the hydrolysis step without further purification.

### Step 2: Preparation of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylic acid, 63

A mixture of methyl 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylate, 62 (790 mg, 1.9 mmol), and LiOH H₂O (0.8 g, 19 mmol) in a mixture of THF (5 ml), methanol (2 ml) and water (2 ml) was stirred at room temperature for 5 h. The mixture was concentrated to dryness and the residue was acidified with acetic acid (2 ml) and loaded onto a column packed with silica/CHCl₃. The column was eluted with 10-40% methanol in CHCl₃. The fractions containing the product 63 were collected and concentrated to give a white solid (474 mg, 61%). ¹H NMR (CD₃OD): 8.14 (s, 1H), 7.86 (d, 1H), 7.51 (s, 1H), 7.43 (d, 1H), 6.30 (s, 1H), 4.16 (m, 1H), 4.02 (m, 2H), 3.82 (m, 1H), 0.88 (m, 3H); LC-MS: 407.1 (M+1).

### Examples 45, 46 and 47

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N,N-dimethylthiophene-3-carboxamide, 64; 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(3-methylbut-2-enyl)thiophene-3-carboxamide, 65; and 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(prop-2-ynyl)thiophene-3-carboxamide, 66

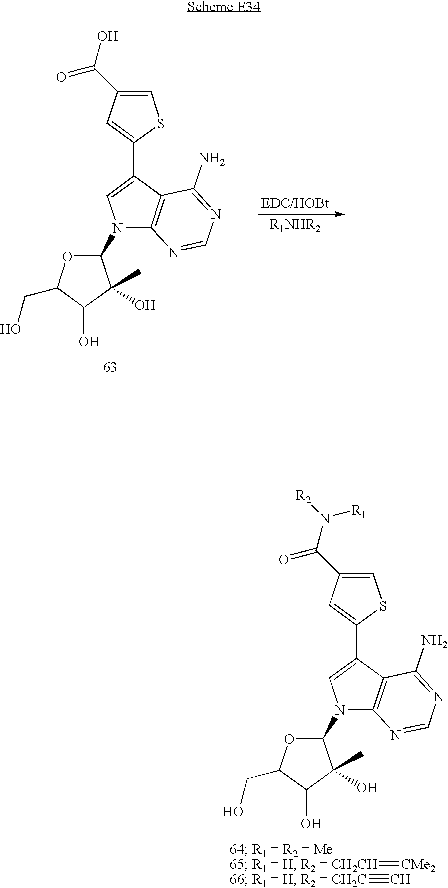

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N,N-dimethylthiophene-3-carboxamide, 64

A mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylic acid, 63 (150 mg, 0.36 mmol), EDC (83 mg, 0.43 mmol), HOBt (64 mg, 0.47 mmol) and DIEA (115 mg, 0.9 mmol) in DMF was stirred for 1 h at room temperature. A solution of dimethylamine in THF (1 ml, 1 mmol, 1.0 M solution) was added and the mixture was stirred at room temperature for 16 h. The mixture was concentrated to dryness and the residue was loaded onto a column packed with silica/CHCl₃. The column was eluted with 5-30% methanol in CHCl₃. The product 64 was isolated as a mixture with EDC/HOBt by-product. The mixture was then taken up in 1M aqueous HCl solution and extracted with EtOAc. The EtOAc layer containing the impurity was discarded. The pH of the aqueous layer was adjusted to ˜8 with aqueous K₂CO₃ and extracted with EtOAc. The organics were concentrated to afford 64 as a white solid (7 mg, 5%). ¹H NMR (CD₃OD): 8.16 (s, 1H), 7.83 (s, 1H), 7.70 (d, 1H), 7.23 (d, 1H), 6.30 (s, 1H), 4.15 (m, 1H), 4.05 (m, 2H), 3.82 (m, 1H), 3.19 (m, 3H), 3.10 (s, 3H), 0.88 (m, 3H); LC-MS: 434.2 (M+1).

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(3-methylbut-2-enyl)thiophene-3-carboxamide, 65

A mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylic acid, 63 (150 mg, 0.36 mmol), EDC (83 mg, 0.43 mmol), HOBt (64 mg, 0.47 mmol) and DIEA (0.23 mg, 1.5 mmol) in DMF was stirred for 1 h at room temperature. 3-Methylbut-2-en-1-amine hydrochloride (0.1 g, 0.8 mmol) was added to the mixture and the mixture was stirred at room temperature for 16 h. The mixture was concentrated to dryness and the residue was loaded onto a column packed with silica/CHCl₃. The column was eluted with 5-30% methanol in CHCl₃. The product 65 was isolated as a mixture with EDC/HOBt by-product. The mixture was then taken up in 1M aqueous HCl solution and extracted with EtOAc. The EtOAc layer containing the impurity was discarded. The pH of the aqueous layer was adjusted to ˜8 with aqueous K₂CO₃ and extracted with EtOAc. The organics were concentrated to afford 65 as a white solid (19.2 mg, 11%). ¹H NMR (CD₃OD): 8.16 (s, 1H), 8.01 (d, 1H), 7.80 (s, 1H), 7.48 (d, 1H), 6.30 (s, 1H), 5.28 (m, 1H), 4.15 (m, 1H), 4.06 (m, 4H), 3.82 (m, 1H), 1.74 (s, 6H), 0.88 (m, 3H); LC-MS: 474.2 (M+1).

### Preparation of 5-(4-amino-7-((2R,3R)-3,4-dihydroxy-5-(hydroxymethyl)-3-methyl tetrahydrofuran-2-yl)-7H-pyrrolo[2,3-d]pyrimidin-5-yl)-N-(prop-2-ynyl)thiophene-3-carboxamide, 66

A mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-3-carboxylic acid, 63 (150 mg, 0.36 mmol), EDC (83 mg, 0.43 mmol), HOBt (64 mg, 0.47 mmol) and DIEA (115 mg, 0.9 mmol) in DMF was stirred for 1 h at room temperature. Propargylamine (0.5 ml) was added and the mixture was stirred at room temperature for 16 h. The mixture was concentrated to dryness and the residue was loaded onto a column packed with silica/CHCl₃. The column was eluted with 5-30% methanol in CHCl₃. The product 66 was isolated as a mixture with EDC/HOBt by-product. The mixture was then taken up in 1M aqueous HCl solution and extracted with EtOAc. The EtOAc layer containing the impurity was discarded. The pH of the aqueous layer was adjusted to ˜8 with aqueous K₂CO₃ and extracted with EtOAc. The organics were concentrated to afford a yellow solid. LC-MS analysis of the yellow solid revealed a mixture of 2 products with identical mass (M+1=444) corresponding to the required product 66.

### Example 48

### Preparation of 5-(4-amino-7-((2R,3R)-3,4-dihydroxy-5-(hydroxymethyl)-3-methyl tetra hydrofuran-2-yl)-7H-pyrrolo[2,3-d]pyrimidin-5-yl)-N-methylthiophene-2-carboxamide, 67

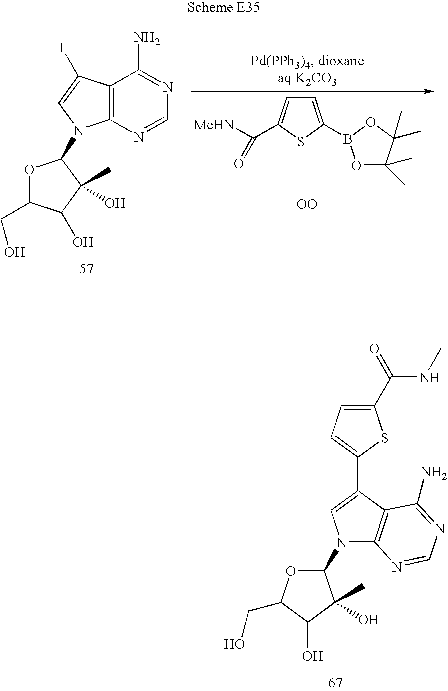

### Preparation of 5-(4-amino-7-((2R,3R)-3,4-dihydroxy-5-(hydroxymethyl)-3-methyl tetra hydrofuran-2-yl)-7H-pyrrolo[2,3-d]pyrimidin-5-yl)-N-methylthiophene-2-carboxamide, 67

A mixture of (2R,3R)-2-(4-amino-5-iodo-7H-pyrrolo[2,3-d]pyrimidin-7-yl)-5-(hydroxyl methyl)-3-methyltetrahydrofuran-3,4-diol 57 (203 mg, 0.5 mmol), N-methyl-5-(4,4,5,5-tetramethyl-1,3,2-dioxaborolan-2-yl)thiophene-2-carboxamide, OO (400 mg crude), Pd(PPh₃)₄ (58 mg, 0.05 mmol), and KOAc (147 mg, 1.5 mmol) was purged with nitrogen for 10 min. 1,4-Dioxane (5 ml) was added and the mixture was stirred at 80° C. for 16 h. LC-MS analysis revealed starting material 57, product 67 and de-iodinated product. The mixture was cooled to ambient temperature and concentrated to dryness in vacuo. The residue was dissolved in a minimal amount of CH₂Cl₂/MeOH (3:1), applied to a column packed with silica/CH₂Cl₂ and eluted with CH₂Cl₂/MeOH (9:1→3:1). Fractions containing product were collected and concentrated to give orange solid. 1H NMR of the orange solid showed only 70% purity (HPLC showed ˜90% purity). The mixture was chromatographed again two times by silica gel chromatography and eluted with CH₂Cl₂/MeOH (9:1→3:1). The fractions were analyzed by HPLC. Fractions containing only the product were concentrated to give 10.4 mg of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)-N-methylthiophene-2-carboxamide, 67 (96% purity by HPLC, ˜95% purity by ¹H NMR). ¹H NMR (300 MHz, CD₃OD): δ 8.17 (d, 1H), 7.87 (s, 1H), 7.65 (dd, 1H), 7.13 (dd, 1H), 6.3 (d, 1H), 4.15 (m, 1H), 4.03 (m, 3H), 3.84 (m, 2H), 2.90 (s, 3H), 0.87 (s, 3H); MS: 420.1 (M+1); calcd for C₁₈H₂₁N₅O₅S: 419.45.

A second component that was a mixture of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)-N-methylthiophene-2-carboxamide, 67 and de-iodinated product 78 mg (˜85% pure by HPLC) was also collected.

### Example 49

### Preparation of 5-(4-amino-7-((2R,3R)-3,4-dihydroxy-5-(hydroxymethyl) tetrahydrofuran-2-yl)-7H-pyrrolo[2,3-d]pyrimidin-5-yl)-N-methylthiophene-2-carboxamide, 68

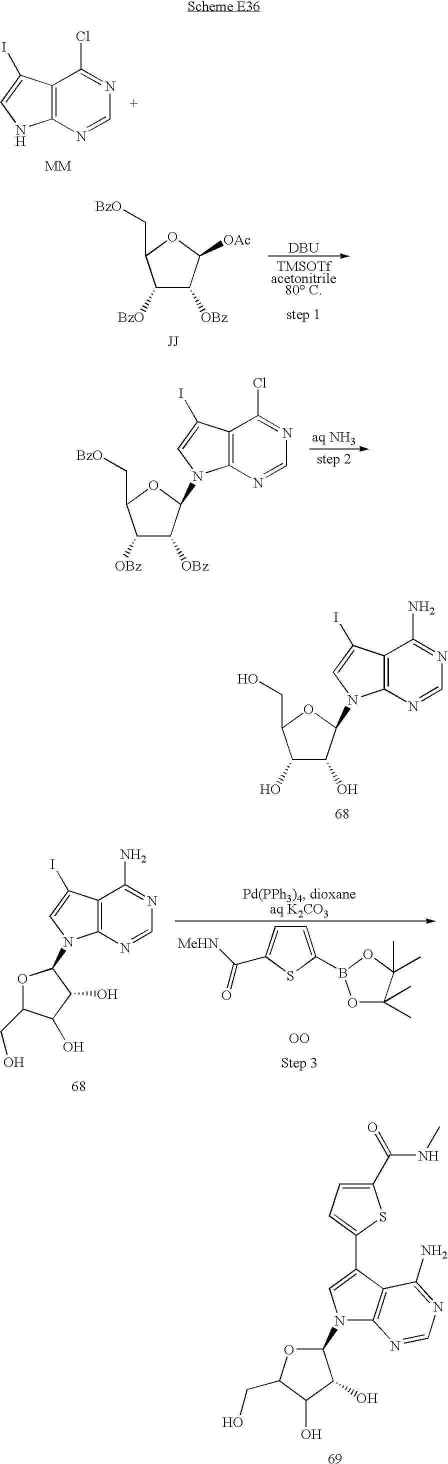

### Step 1: Preparation of (2R,3R,4R,5R)-2-(benzoyloxymethyl)-5-(4-chloro-5-iodo-7H-pyrrolo[2,3-d]pyrimidin-7-yl)tetrahydrofuran-3,4-diyl dibenzoate

To a suspension of MM (6.7 g, 24.0 mmol) and JJ (12 g, 24.0 mmol) in anhydrous acetonitrile (240 ml) was added DBU (5.4 ml, 36.0 mmol). The mixture was then treated with TMSOTf (8.7 ml, 48.0 mmol). The mixture was stirred at room temperature for 30 min and then heated at 80° C. for 17 h. The mixture was cooled to room temperature. A saturated aqueous NaHCO₃ solution (250 ml) was added to the reaction mixture and extracted with EtOAc (2×200 ml). The organics were dried over Na₂SO₄ and concentrated to give orange residue.

The orange residue was triturated with methanol to give brown solid. The brown solid was dissolved in minimal amount of CH₂Cl₂ and loaded onto a column packed with silica/CH₂Cl₂ and eluted with CH₂Cl₂/EtOAc (2:1). The fractions containing the required product were collected and concentrated to afford (2R,3R,4R,5R)-2-(benzoyloxymethyl)-5-(4-chloro-5-iodo-7H-pyrrolo[2,3-d]pyrimidin-7-yl)tetrahydrofuran-3,4-diyl dibenzoate as off-white solid (10.9 g, 63%). LC/MS m/z 724 (M+H).

### Step 2: Preparation of (2R,3R,4S,5R)-2-(4-amino-5-iodo-7H-pyrrolo[2,3-d]pyrimidin-7-yl)-5-(hydroxymethyl)tetrahydrofuran-3,4-diol, 68

A mixture of (2R,3R,4R,5R)-2-(benzoyloxymethyl)-5-(4-chloro-5-iodo-7H-pyrrolo[2,3-d]pyrimidin-7-yl)tetrahydrofuran-3,4-diyl dibenzoate, (3.0 g, 4.1 mmol), and aqueous NH₃ (45 ml) in 1,4-dioxane (45 ml) was stirred at 80° C. for 16 h. LC-MS analysis indicated complete reaction. The mixture was concentrated to dryness. The residue was suspended in CH₂Cl₂/MeOH (9:1) and loaded onto a column packed with silica/CH₂Cl₂. The column was eluted with CH₂Cl₂/MeOH (9:1→1:1). The fractions containing product were collected and concentrated to (2R,3R,4S,5R)-2-(4-amino-5-iodo-7H-pyrrolo[2,3-d]pyrimidin-7-yl)-5-(hydroxymethyl)tetrahydrofuran-3,4-diol, 68 as a white solid (1.1 g, 69%). ¹H NMR (DMSO-d₆) δ 8.10 (s, 1H), 7.68 (s, 1H), 6.68 (br s, 2H), 6.03 (d, 1H), 5.33 (br s, 1H), 5.15 (br, 2H), 4.36 (br s, 1H), 4.07 (br s, 1H), 3.88 (m, 1H), 3.56 (m, 2H).

### Step 3: Preparation of 5-(4-amino-7-((2R,3R)-3,4-dihydroxy-5-(hydroxymethyl)tetrahydrofuran-2-yl)-7H-pyrrolo[2,3-d]pyrimidin-5-yl)-N-methylthiophene-2-carboxamide, 69

A mixture of (2R,3R)-2-(4-amino-5-iodo-7H-pyrrolo[2,3-d]pyrimidin-7-yl)-5-(hydroxymethyl)tetra hydrofuran-3,4-diol, 68 (393 mg, 1.0 mmol), N-methyl-5-(4,4,5,5-tetramethyl-1,3,2-dioxaborolan-2-yl)thiophene-2-carboxamide, OO (crude ca. 614 mg, 2.3 mmol), Pd(PPh₃)₄ (59 mg, 0.05 mmol), and KOAc (147 mg, 1.5 mmol) was purged with nitrogen for 10 min. 1,4-Dioxane (10 ml) was added and the mixture was stirred at 80° C. for 16 h. LC-MS analysis revealed ˜20% product 5. The mixture was cooled to ambient temperature.

The mixture was concentrated to dryness. The residue was applied to a column packed with silica/CH₂Cl₂ and eluted with 5-40% methanol in chloroform to give 171 mg (55%) of 69 as a mixture with starting material 68 and de-iodinated product. The mixture was further purified by preparative HPLC to afford 11 mg (5%) of 5-(4-amino-7-((2R,3R)-3,4-dihydroxy-5-(hydroxymethyl)tetrahydrofuran-2-yl)-7H-pyrrolo[2,3-d]pyrimidin-5-yl)-N-methylthiophene-2-carboxamide, 69. LC/MS m/z 406.2 (M+H). ¹H NMR (CD₃OD) δ 8.33 (s, 1H), 7.95 (s, 1H), 7.67 (d, 1H), 7.19 (d, 1H), 6.30 (d, 1H), 4.70 (m, 1H), 4.30 (m, 1H), 4.12 (m, 1H), 3.81 (qd, 2H), 2.91 (s, 3H).

### Example 50

### Preparation of (4R,5R)-5-(4-amino-5-(thiophen-2-yl)-7H-pyrrolo[2,3-d]pyrimidin-7-yl)-4-fluoro-2-(hydroxymethyl)-4-methyltetrahydrofuran-3-ol, 71

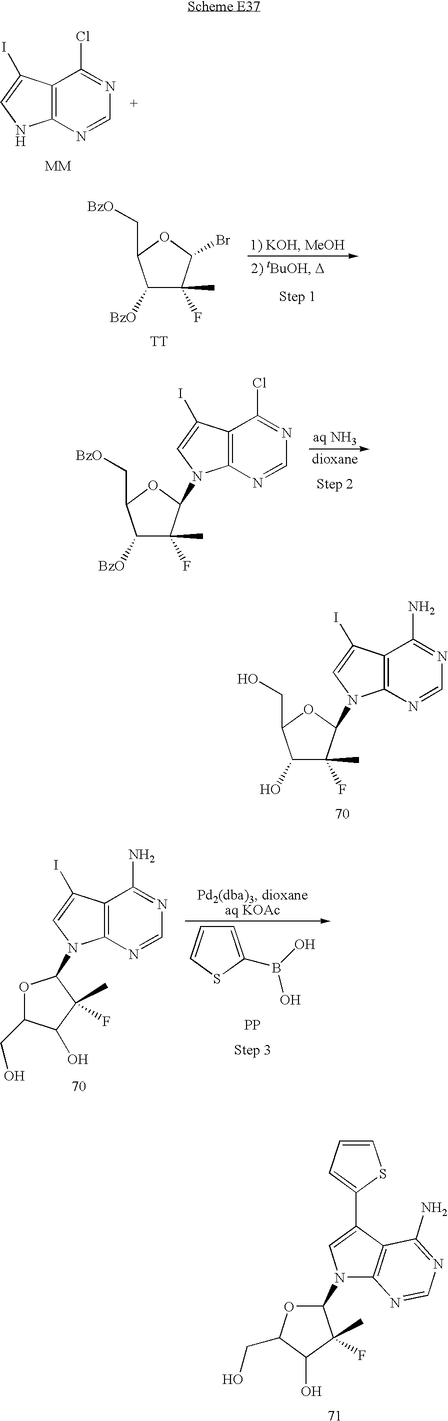

Step 1: Preparation of ((2R,3R,4R,5R)-3-(benzoyloxy)-5-(4-chloro-5-iodo-7H-pyrrolo[2,3-d]pyrimidin-7-yl)-4-fluoro-4-methyltetrahydrofuran-2-yl)methyl benzoate

A solution of MM (28 g, 101 mmol) in methanol was added powdered KOH (5.7 g, 101 mmol). The mixture was stirred at ambient temperature for 2 h and concentrated to dryness. The residue was suspended in toluene and concentrated again to remove any water. The solid residue was then added to compound TT (29 g, 67 mmol) in t-BuOH (500 ml). The mixture was stirred at 50° C. for 6 days. LC-MS revealed ˜25% starting material TT remained. The mixture was cooled to room temperature and concentrated to a orange solid. The orange solid was loaded onto a column packed with silica/hexanes. The column was eluted with hexanes/EtOAc (9:1→8:2). The fractions containing required product were collected and concentrated to give intermediate ((2R,3R,4R,5R)-3-(benzoyloxy)-5-(4-chloro-5-iodo-7H-pyrrolo[2,3-d]pyrimidin-7-yl)-4-fluoro-4-methyltetrahydrofuran-2-yl)methyl benzoate as a pale-yellow solid. The solid was triturated with methanol to afford white solid (5.3 g, 12%).

### Step 2: Preparation of (2R,3R,4R,5R)-5-(4-amino-5-iodo-7H-pyrrolo[2,3-d]pyrimidin-7-yl)-4-fluoro-2-(hydroxymethyl)-4-methyltetrahydrofuran-3-ol, 70

To a solution of ((2R,3R,4R,5R)-3-(benzoyloxy)-5-(4-chloro-5-iodo-7H-pyrrolo[2,3-d]pyrimidin-7-yl)-4-fluoro-4-methyltetrahydrofuran-2-yl)methyl benzoate, ? (5.3 g, 8.3 mmol) in 1,4-dioxane (80 ml) was added aq NH₃ (28-30%, 200 ml). The mixture was stirred at 90° C. for 3 days. LC-MS indicated complete reaction. The mixture was cooled to room temperature, and concentrated to dryness. The residue was suspended in EtOAc/MeOH (10/5 ml) and loaded onto a column packed with silica/EtOAc. Eluted with EtOAc→EtOAc/MeOH (9:1→6:4). The fractions containing pure product were collected and concentrated followed by trituration with EtOAc to give (2R,3R,4R,5R)-5-(4-amino-5-iodo-7H-pyrrolo[2,3-d]pyrimidin-7-yl)-4-fluoro-2-(hydroxymethyl)-4-methyltetrahydrofuran-3-ol, 70 as a yellow solid (2.85 g, 83%). ¹H NMR (CD₃OD) δ 8.12 (s, 1H), 7.77 (s, 1H), 6.40 (d, 1H), 4.23 (m, 1H), 4.02 (m, 2H), 3.83 (dd, 1H), 1.03 (d, 3H). LC/MS m/z 408.9 (M+H).

### Step 3: Preparation of (4R,5R)-5-(4-amino-5-(thiophen-2-yl)-7H-pyrrolo[2,3-d]pyrimidin-7-yl)-4-fluoro-2-(hydroxymethyl)-4-methyltetrahydrofuran-3-ol, 71

A mixture of (4R,5R)-5-(4-amino-5-iodo-7H-pyrrolo[2,3-d]pyrimidin-7-yl)-4-fluoro-2-(hydroxymethyl)-4-methyltetrahydrofuran-3-ol, 70 (250 mg, 0.612 mmol), thiophen-2-ylboronic acid, PP (117 mg, 0.918 mmol), Pd₂(dba)₃ (56 mg, 0.06 mmol), and KOAc (120 mg, 1.2 mmol) was purged with nitrogen for 10 min. 1,4-Dioxane (15 ml) and water (5 ml) were added and the mixture was stirred at 80° C. for 16 h. LC-MS analysis revealed a mixture of desired product 71, starting material 70 and de-iodinated product. The mixture was cooled to ambient temperature and quenched with water. The mixture was filtered through a plug of celite and extracted with ethyl acetate (2×50 ml). The organic phase was dried over sodium sulfate and concentrated to dryness. The residue was applied to a column and eluted with ethyl acetate/hexanes (20% to 50%). The fractions containing compound 71 were combined and concentrated to dryness to give a ˜85% pure desired product 71 contaminated with starting material 70. The mixture was dissolved in methanol and hydrogenated at 1 atm H₂ in the presence of Pd/C for 2 hours to cleave the iodide from the starting material. The mixture was filtered through a plug of celite and concentrated to dryness. The residue was applied to a column and eluted with ethyl acetate to give 35 mg of (4R,5R)-5-(4-amino-5-(thiophen-2-yl)-7H-pyrrolo[2,3-d]pyrimidin-7-yl)-4-fluoro-2-(hydroxylmethyl)-4-methyltetrahydrofuran-3-ol, 71 as an orange solid (16%, 95% purity). LC/MS m/z 365.1 (M+H). ¹H NMR (CD₃OD) δ 8.17 (s, 1H), 7.71 s, 1H), 7.46 (m, 1H), 7.16 (m, 1H), 6.46 (d, 1H), 4.28 (m, 1H), 4.06 (m, 2H), 3.84 (m, 1H), 1.09 (d, 3H).

### Example 51

### Preparation of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carbonitrile, 72

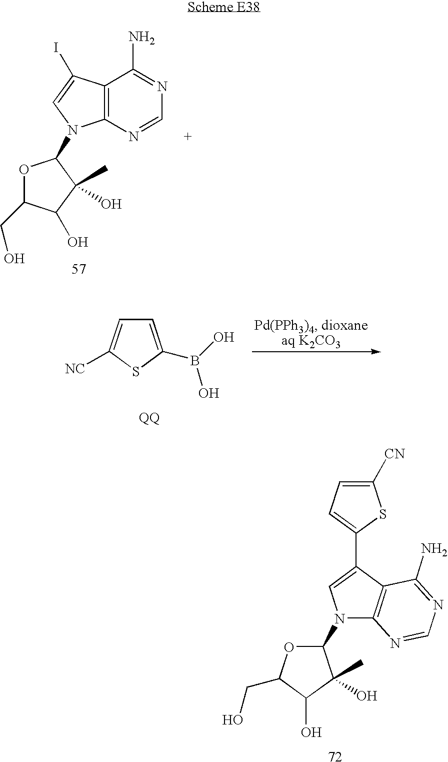

### Preparation of 5-(4-amino-7-((2R,3R)-3,4-dihydroxy-5-(hydroxymethyl)-3-methyltetrahydrofuran-2-yl)-7H-pyrrolo[2,3-d]pyrimidin-5-yl)thiophene-2-carbonitrile, 72

A mixture of 57 (812 mg, 2.0 mmol), QQ (459 mg, 3.0 mmol), Pd(PPh₃)₄ (231 mg, 0.2 mmol) and KOAc (588 mg, 6.0 mmol) was purged with nitrogen for 10 min. 1,4-Dioxane (20 ml) was added to the mixture and the mixture was stirred at 80° C. for 16 h. LC-MS analysis revealed ˜35% product 3 formation. Water was added to this mixture and stirred at 80° C. for 2 h. LC-MS analysis revealed a mixture of starting material 57, product 72 and de-iodinated product in the ratio of 2:1:2. The mixture was concentrated to dryness. The residue was loaded onto a column packed with silica/CHCl₃. The column was eluted with 5-10% methanol in CHCl₃. The product 72 was isolated as a mixture with starting material 57 and some other impurities.

### Examples 52-56

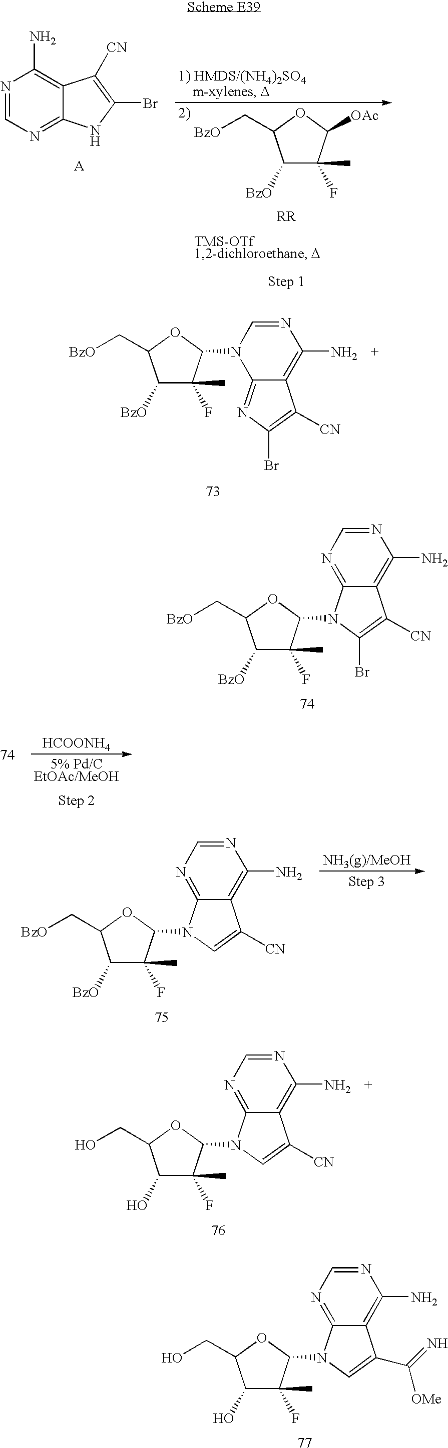

### Example 57

### Methyl-amino-7-((2S,3R,4R)-3-fluoro-4-hydroxy-5-(hydroxymethyl)-3-methyltetrahydrofuran-2-yl)-7H-pyrrolo[2,3-d]pyrimidine-5-carbimidate (77)

### Step 1: Preparation of (3R,4R,5S)-5-(4-amino-6-bromo-5-cyano-1H-pyrrolo[2,3-d]pyrimidin-1-yl)-2-(benzoyloxymethyl)-4-fluoro-4-methyltetrahydrofuran-3-yl benzoate (73) and (3R,4R,5S)-5-(4-amino-6-bromo-5-cyano-7H-pyrrolo[2,3-d]pyrimidin-7-yl)-2-(benzoyloxymethyl)-4-fluoro-4-methyltetrahydrofuran-3-yl benzoate (74)

A mixture of 4-amino-6-bromo-5-cyanopyrrolo[2,3-d]pyrimidine, A (3.3 g, 0.014 mol), hexamethyldisilizane (250 mL), ammonium sulfate (0.16 g, 0.0012 mol) and m-xylenes (80 mL) were heated at 130° C. for 20 hr. The reaction mixture was concentrated in vacuo and m-xylenes (20 mL) was added to the residue. The mixture was concentrated in vacuo and the residue was dried under vacuum for 1 hr. The residue was dissolved in dichloroethane (225 mL) and (3R,4R)-5-acetoxy-2-(benzoyloxymethyl)-4-fluoro-4-methyltetrahydrofuran-3-yl benzoate RR (5.0 g, 0.012 mol) was added. The mixture was chilled to 10° C. and trimethylsilyl trifluoromethanesulfonate (5.3 g, 0.024 mol) was added dropwise over a 15 min. period after which the mixture was heated at reflux for 29 hr. The mixture was allowed to cool to room temperature and was poured into a solution of sodium bicarbonate (8.1 g, 0.096 mol) in water (100 mL) After stirring at room temperature for 30 min., the mixture was extracted with chloroform (had to filter through celite to remove some flocculent solids). The combined organic phase was washed with water, dried over sodium sulfate and concentrated in vacuo. The residue was purified on a silica gel column using a gradient of hexanes to 40% EtOAc/hexanes. Obtained three components. Component A consisting of the pure faster running material (Rf=0.41, TLC 40% EtOAc/hexanes), Component A′ consisting of the faster running material plus impurities and Component B consisting of the pure slower running material (Rf=0.18, TLC 40% EtOAc/hexanes). Component A′ was rechromatographed and the pure Component A was combined with Component A from above to give a total of 1.79 g (25% yield) of A as an off-white solid. Component B amounted to 0.44 g (6% yield) of a pale yellow solid.

Component A was identified as by NMR analysis to be (3R,4R,5S)-5-(4-amino-6-bromo-5-cyano-1H-pyrrolo[2,3-d]pyrimidin-1-yl)-2-(benzoyloxymethyl)-4-fluoro-4-methyltetrahydrofuran-3-yl benzoate, 73. ¹H NMR (d₆-DMSO) δ 8.7 (br s, 1H), 8.66 (d, 1H), 7.93-7.99 (m, 4H), 7.72-7.64 (m, 1H), 7.51-7.61 (m, 3H), 7.38-7.59 (m, 3H), 7.06 (d, 1H), 5.96 (dd, 1H), 5.11 (m, 1H), 4.62 (m, 2H), 1.53 (d, 3H).

Component B was identified by NMR analysis to be (3R,4R,5S)-5-(4-amino-6-bromo-5-cyano-7H-pyrrolo[2,3-d]pyrimidin-7-yl)-2-(benzoyloxymethyl)-4-fluoro-4-methyltetrahydrofuran-3-yl benzoate, 74.

¹H NMR (d₆-DMSO) δ 8.24 (s, 1H), 7.90-7.99 (m, 4H), 7.66-7.72 (m, 1H), 7.58-7.50 (m, 3H), 7.41 (m, 2H), 7.02 (d, 1H), 7.03 (br s, 2H), 5.95 (dd, 1H), 5.14 (m, 1H), 4.61 (m, 2H), 1.48 (d, 3H).

### Step 2: (3R,4R,5S)-5-(4-amino-5-cyano-7H-pyrrolo[2,3-d]pyrimidin-7-yl)-2-(benzoyloxymethyl)-4-fluoro-4-methyltetrahydrofuran-3-yl benzoate (75)

To a solution of (3R,4R,5S)-5-(4-amino-6-bromo-5-cyano-7H-pyrrolo[2,3-d]pyrimidin-7-yl)-2-(benzoyloxymethyl)-4-fluoro-4-methyltetrahydrofuran-3-yl benzoate (74) (0.37 g, 0.0007 mol) in ethyl acetate (4 mL) at room temperature was added ammonium formate (0.42 g, 0.0007 mol) and 5% Pd/C (0.04 g). Methanol (4 mL) was added and the reaction mixture was heated at reflux for 18 hr. The reaction mixture was cooled to room temperature and filtered through celite. The organic phase was washed with water and brine and dried over sodium sulfate. The mixture was filtered and the filtrate was concentrated in vacuo. The residue was purified by silica gel column chromatography using a gradient of hexanes to 40% ethyl acetate/hexanes. The fractions containing the pure major component with R_{f}=0.23 (40% ethyl acetate/hexanes) were concentrated in vacuo to give 0.28 g (82%) of (3R,4R,5S)-5-(4-amino-5-cyano-7H-pyrrolo[2,3-d]pyrimidin-7-yl)-2-(benzoyloxymethyl)-4-fluoro-4-methyl tetra hydrofuran-3-yl benzoate (75) as a colorless glass. ¹H NMR (d₆-DMSO) δ 8.45 (d, 1H), 8.25 (s, 1H), 7.96 (dd, 4H), 7.70 (m, 1H), 7.57 (m, 3H), 7.42 (m, 2H), 6.64 (br s, 2H), 6.83 (d, 1H), 5.93 (dd, 1H), 5.00 (m, 1H), 4.61 (m, 2H), 1.46 (d, 3H).

### Step 3: Preparation of 4-amino-7-((2S,3R,4R)-3-fluoro-4-hydroxy-5-(hydroxymethyl)-3-methyl tetra hydro furan-2-yl)-7H-pyrrolo[2,3-d]pyrimidine-5-carbonitrile (76) and methyl 4-amino-7-((2S,3R,4R)-3-fluoro-4-hydroxy-5-(hydroxymethyl)-3-methyltetrahydrofuran-2-yl)-7H-pyrrolo[2,3-d]pyrimidine-5-carbimidate (77)

A mixture of (3R,4R,5S)-5-(4-amino-5-cyano-7H-pyrrolo[2,3-d]pyrimidin-7-yl)-2-(benzoyloxymethyl)-4-fluoro-4-methyltetrahydrofuran-3-yl benzoate (75) (0.22 g, 0.004 mol) and 7.0N NH₃ in MeOH (12 mL) were placed in a sealed pressure bottle and stirred at room temperature for 18 hr. The mixture was concentrated in vacuo and purified by column chromatography using a stepwise gradient of dichloromethane to 10% MeOH/dichloromethane. The fractions containing the less polar component were combined and concentrated in vacuo to get 0.06 g of 4-amino-7-((2S,3R,4R)-3-fluoro-4-hydroxy-5-(hydroxymethyl)-3-methyl tetra hydro furan-2-yl)-7H-pyrrolo[2,3-d]pyrimidine-5-carbonitrile (76).

FAB-MS m/z 308 (M+H). ¹H NMR (d₆-DMSO) δ 8.22 (s, 2H), 6.89 (br s, 2H), 6.41 (d, 1H), 5.74 (d, 1H), 4.87 (t, 1H), 4.03-4.18 (m, 2H), 3.71 (m, 1H), 3.51 (m, 1H), 1.31 (d, 3H). Combined the fractions containing the more polar component and concentrated in vacuo to get 0.069 g of methyl 4-amino-7-((2S,3R,4R)-3-fluoro-4-hydroxy-5-(hydroxymethyl)-3-methyltetrahydrofuran-2-yl)-7H-pyrrolo[2,3-d]pyrimidine-5-carbimidate (77). FAB-MS m/z 340 (M+H). ¹H NMR (d₆-DMSO) δ 9.95 (d, 1H), 8.17 (s, 1H), 8.06 (s, 1H), 7.57 (d, 1H), 7.26 (d, 1H), 6.38 (d, 1H), 5.67 (d, 1H), 4.88 (t, 1H), 4.01-4.16 (m, 2H), 3.74 (s, 3H), 3.69 (m, 1H), 3.50 (m, 1H), 1.29 (d, 3H).

### Example 58 and 59

### Preparation of ((2R,3R,4R,5R)-3-(benzoyloxy)-5-(5-bromo-4-chloro-7H-pyrrolo[2,3-d]pyrimidin-7-yl)-4-fluoro-4-methyltetrahydrofuran-2-yl)methyl benzoate, 78 and (2R,3R,4R,5R)-5-(4-amino-5-bromo-7H-pyrrolo[2,3-d]pyrimidin-7-yl)-4-fluoro-2-(hydroxymethyl)-4-methyltetrahydrofuran-3-ol, 79.

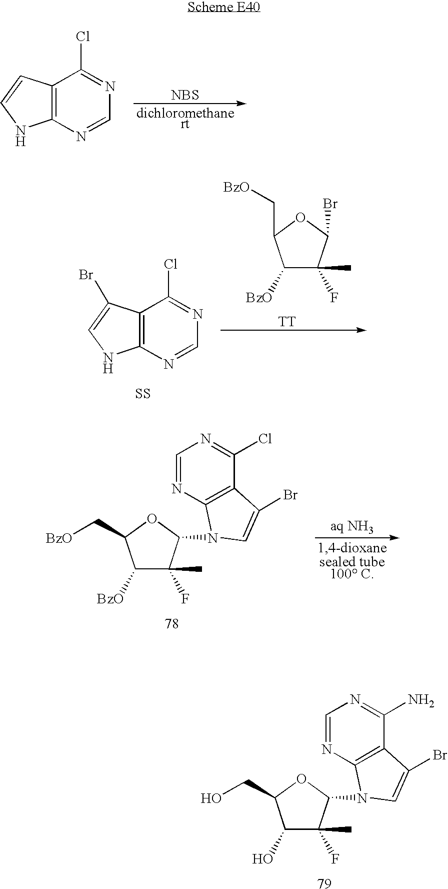

### Preparation of 5-bromo-4-chloro-7H-pyrrolo[2,3-d]pyrimidine, SS

In a 3 L round bottom flask, 4-chloro-7H-pyrrolo[2,3-d]pyrimidine (92 g, 600 mmol) was suspended in 1600 mL dichloromethane; NBS (108 g, 600 mmol) was gradually added and the mixture was stirred at room temperature for 1 hr. An additional amount of NB S (20 g, 56 mmol) was added and the mixture was stirred at room temperature for 2 hours. The resulting solid was collected via filtration, rinsed with dichloromethane and dried. The solid was triturated with 2 L of water for 2 hours and the solid was collected via filtration; The solid was dried under a vacuum to a constant weight (112 g, 80%). ¹H NMR (DMSO-d₆) δ 8.68 (s, 1H), 7.99 (s, 1H).

### Preparation of ((2R,3R,4R,5S)-3-(benzoyloxy)-5-(5-bromo-4-chloro-7H-pyrrolo[2,3-d]pyrimidin-7-yl)-4-fluoro-4-methyltetrahydrofuran-2-yl)methyl benzoate, 78

To a solution of 5-bromo-4-chloro-7H-pyrrolo[2,3-d]pyrimidine, SS (230 mg, 1 mmol) in methanol (10 ml) was added powdered KOH (56 mg, 1 mmol). The mixture was stirred at ambient temp for 1 h and concentrated to dryness. The residue was suspended in CH₃CN and concentrated again to remove any water. The residue was then taken in DMF (5 ml) and a solution of compound TT (436 mg, 1 mmol) in DMF (5 ml) was added. The mixture was stirred at 60° C. for 2 h. The mixture was cooled to room temperature and water (50 ml) was added. The mixture was extracted with EtOAc (2×50 ml), the combined organic phase was dried over Na₂SO₄ and concentrated to give a crude residue. The residue was dissolved in a minimal amount of EtOAc and loaded onto a column packed with silica/CH₂Cl₂. The column was eluted with CH₂Cl₂/EtOAc (9:1→8:2). The fractions containing the product were collected and concentrated to give ((2R,3R,4R,5R)-3-(benzoyloxy)-5-(5-bromo-4-chloro-7H-pyrrolo[2,3-d]pyrimidin-7-yl)-4-fluoro-4-methyltetrahydrofuran-2-yl)methyl benzoate, 78 as an orange solid (230 mg, 39%, 85% pure by HPLC). This material was used for the next step without further purification. ¹H NMR (CDCl₃): 8.62 (s, 1H), 7.9-8.2 (m, 4H), 7.3-7.7 (m, 7H), 6.79 (d, 1H), 5.77 (dd, 1H), 4.89 (m, 1H), 4.52-76 (m, 2H), 3.51 (m, 2H), 1.50 (m, 3H); LC-MS: 588.0 and 590.1 (M+1).

### Preparation of (2R,3R,4R,5S)-5-(4-amino-5-bromo-7H-pyrrolo[2,3-d]pyrimidin-7-yl)-4-fluoro-2-(hydroxymethyl)-4-methyltetrahydrofuran-3-ol, 79

A mixture of ((2R,3R,4R,5R)-3-(benzoyloxy)-5-(5-bromo-4-chloro-7H-pyrrolo[2,3-d]pyrimidin-7-yl)-4-fluoro-4-methyltetrahydrofuran-2-yl)methyl benzoate, 78 (210 mg, 0.36 mmol), and aqueous NH₃ (30 ml) in 1,4-dioxane (15 ml) was stirred at 100° C. for 20 h in a sealed tube. The mixture was cooled to ambient temperature and concentrated to dryness in vacuo. The residue was suspended in CH₂Cl₂/MeOH (9:1) and loaded onto a column packed with silica/CH₂Cl₂. The column was eluted with CH₂Cl₂/MeOH (95:5). The fractions containing the product were collected and concentrated to a white solid (0.8 g, containing some inorganic salt). The mixture was then taken in water (50 ml) and extracted with EtOAc (2×50 ml). The organics were dried over Na₂SO₄ and concentrated to give 79, (2R,3R,4R,5R)-5-(4-amino-5-bromo-7H-pyrrolo[2,3-d]pyrimidin-7-yl)-4-fluoro-2-(hydroxymethyl)-4-methyltetrahydrofuran-3-ol as a white solid (86 mg, 66%). ¹H NMR (CD₃OD): 8.10 (s, 1H), 7.36 (d, 1H), 6.45 (d, 1H), 4.22 (m, 2H), 3.88 (m, 1H), 3.67 (m, 1H), 1.40 (d, 1H, J=21 Hz); LC-MS: 360.9 and 363.0 (M+1).

### Example 60, 61 and 62

### Preparation of [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-(3-(hexadecyloxy)propyl)cyclic phosphate 95; [5-amino-7-((2R,3S,4S,5R)-3-azido-4-hydroxy-5-(hydroxymethyl)tetrahydrofuran-2-yl)-7H-pyrrolo[2,3-d]pyrimidine-4-carboxamide]-3′,5′-(3-(hexadecyloxy)propyl)cyclic phosphate, 96; and [(2R,3R,4S,5R)-2-(5-((E)-1-amino-2-hydroxyvinyl)-4-(3-methylbut-2-enylamino)-7H-pyrrolo[2,3-d]pyrimidin-7-yl)-5-(hydroxymethyl)tetrahydrofuran-3,4-diol]-3′,5′-(3-(hexadecyloxy)propyl)cyclic phosphate, 97

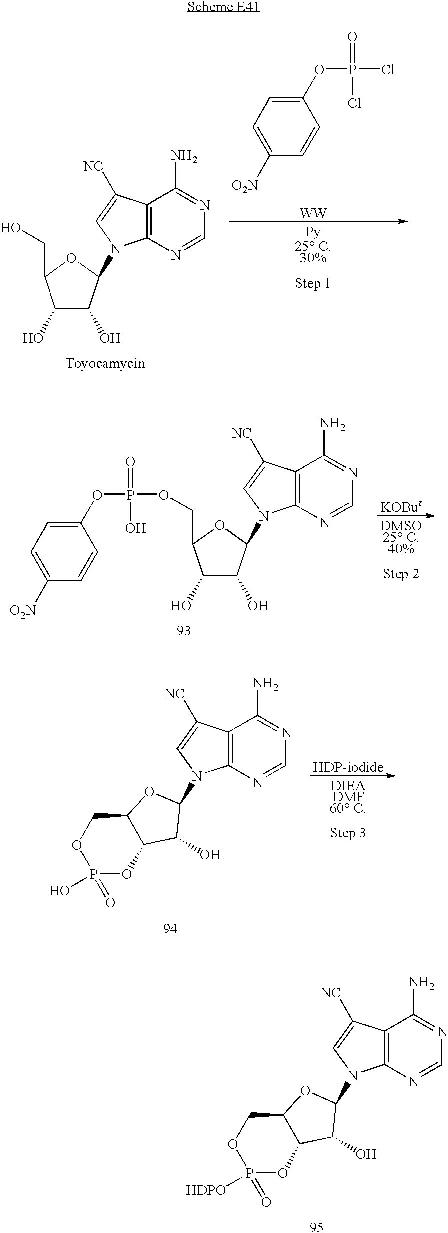

### Step 1: Preparation of ((3S,4R,5R)-5-(4-amino-5-cyano-7H-pyrrolo[2,3-d]pyrimidin-7-yl)-3,4-dihydroxytetrahydrofuran-2-yl)methyl 4-nitrophenyl hydrogen phosphate, 93

To a clear solution of toyocamycin (0.29 g, 1 mmol, 1 equiv.) in anhydrous pyridine (12 mL), was added 4-nitrophenyl phosphorodichloridate, WW (0.26 g, 1 mmol, 1 equiv.) in anhydrous pyridine (6 mL) at 25° C. The reaction mixture was stirred at 25° C. overnight. The reaction was stopped, quenched with 1 mL of water and stirred for 15 min. Volatiles were removed in vacuo and azeotroped with toluene to remove residual pyridine and water. Silica gel column purification of the residue (5-50% MeOH/CH₂Cl₂) afforded 0.16 g of impure 93. Continued elution with 50% MeOH/CH₂Cl₂-100% MeOH-50% MeOH/NH₄OH-100% NH₄OH eluted 0.1 g (30%) of pure ((3S,4R,5R)-5-(4-amino-5-cyano-7H-pyrrolo[2,3-d]pyrimidin-7-yl)-3,4-dihydroxytetrahydrofuran-2-yl)methyl 4-nitrophenyl hydrogen phosphate, 93 as a brown solid. LC/MS m/z 493.1 (M+1).

### Step 2: Preparation of [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-cyclic phosphate, 94

To 0.1 g of ((3S,4R,5R)-5-(4-amino-5-cyano-7H-pyrrolo[2,3-d]pyrimidin-7-yl)-3,4-dihydroxytetra hydro furan-2-yl)methyl 4-nitrophenyl hydrogen phosphate, 93 (0.2 mmol, 1 equiv.) in DMSO (20 mL), 1M KOBu^{t} in t-BuOH (0.6 mmol, 0.6 mL, 3 equiv.) was added at 25° C. The reaction mixture was stirred at 25° C. overnight. Amberlite IR-120 (plus) ion exchange resin (hydrogen form) was added to the reaction mixture to bring down the pH to 6. The resin was filtered and washed with NH₄OH solution. The filtrate was evaporated in vacuo and azeotroped with toluene. The residue was triturated in MeOH and filtered. The filtrate was evaporated in vacuo. The residue was purified by silica gel column chromatography (5-40% MeOH/CH₂Cl₂) to afford 37 mg (40% yield) of slightly impure [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-cyclic phosphate, 94 as an off white solid. LC/MS m/z 354 (M+H).

### Step 3: Preparation of [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-(3-(hexadecyloxy)propyl)cyclic phosphate 95

[4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-cyclic phosphate, 94 (50 mg, 0.14 mmol, 1 equiv.) was placed in triethylamine and evaporated in vacuo (2×). This was dissolved in anhydrous DMF (5 mL) and N,N-diisopropylethylamine (0.05 mL, 0.28 mmol, 2 equiv.) was added. The reaction mixture was heated at 60° C. and HDP-iodide (63 mg, 0.15 mmol, 1.1 equiv.) in anhydrous DMF (5 mL) was added dropwise. The reaction mixture was stirred at 60° C. overnight. LC-MS showed the formation of two desired products of same mass in ˜1:1 ratio (m/e: 636, retention times: 5.38, 5.63), their corresponding ring opened products and unreacted 94. N,N-Diisopropylethylamine (0.1 mL, 0.56 mmol, 4 equiv.) and HDP-iodide (126 mg, 0.3 mmol, 2.2 equiv.) was added again and the mixture was stirred at 60° C. for 24 h. The reaction was stopped and the volatiles were removed in vacuo. The residue was purified by column chromatography (0-4% MeOH/CH₂Cl₂) to afford 58 mg of impure 95. A second column purification (50-80% EtOAc/hexanes) afforded 7 mg (8% yield, >95% purity by LC-MS) of [4-amino-7-((2R,3R,4S,5R)-3,4-dihydroxy-5-(hydroxymethyl)tetrahydrofuran-2-yl)-7H-pyrrolo[2,3-d]pyrimidine-5-carbonitrile]-3′,5′-(3-(hexadecyloxy)propyl)cyclic phosphate, 95 as an off white solid as an ˜1:1 ratio of diastereomers at phosphorous. LC/MS m/z 636.4 (Rₜ=5.414) and 636.4 (Rₜ=5.694) (M+H).

The following compounds are made as described above for compound 95.

### [5-amino-7-((2R,3S,4S,5R)-3-azido-4-hydroxy-5-(hydroxymethyl)tetrahydrofuran-2-yl)-7H-pyrrolo[2,3-d]pyrimidine-4-carboxamide]-3′,5′-(3-(hexadecyloxy)propyl)cyclic phosphate, 96

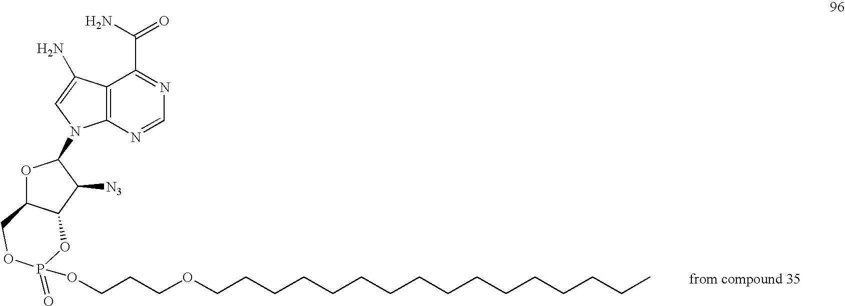

### [(2R,3R,4S,5R)-2-(5-((E)-1-amino-2-hydroxyvinyl)-4-(3-methylbut-2-enylamino)-7H-pyrrolo[2,3-d]pyrimidin-7-yl)-5-(hydroxymethyl)tetrahydrofuran-3,4-diol]-3′,5′-(3-(hexadecyloxy)propyl)cyclic phosphate, 97

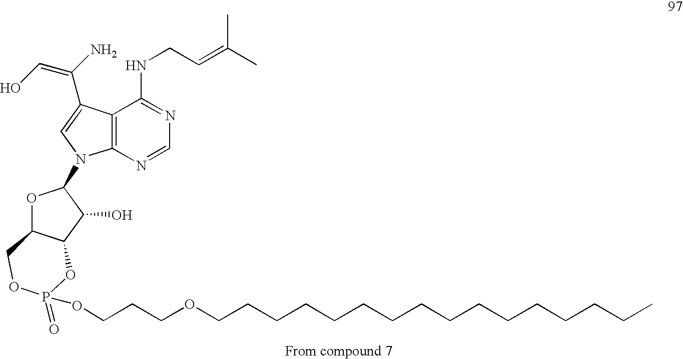

### Example 63

### 4-Amino-7-((2R,3R,4R,5R)-3,4-dihydroxy-5-(hydroxymethyl)-3-methyltetrahydrofuran-2-yl)-7H-pyrrolo[2,3-d]pyrimidine-5-carbonitrile, 88

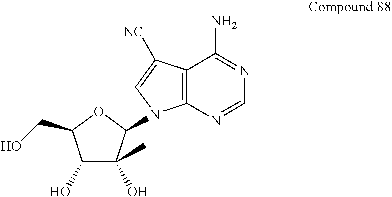

4-Amino-7-((2R,3R,4R,5R)-3,4-dihydroxy-5-(hydroxymethyl)-3-methyltetrahydrofuran-2-yl)-7H-pyrrolo[2,3-d]pyrimidine-5-carbonitrile, 88 was prepared according to Murai, et al., Heterocycles, 1992, vol. 33, #1, 391-404.

### Example 64

### (2R,3R,4S,5R)-2-(4-Amino-5-bromo-7H-pyrrolo[2,3-d]pyrimidin-7-yl)-5-(hydroxymethyl) tetrahydrofuran-3,4-diol, 89

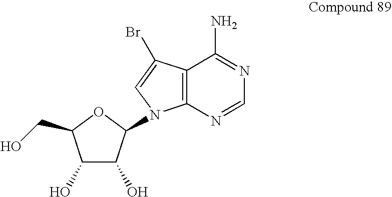

(2R,3R,4S,5R)-2-(4-Amino-5-bromo-7H-pyrrolo[2,3-d]pyrimidin-7-yl)-5-(hydroxymethyl) tetrahydrofuran-3,4-diol, 89 was prepared according to Erion et al., J. Med. Chem., 2003, vol. 46, #22, 4750-4760.

### Example 65

### 4-Amino-7-((2R,3R,4S,5R)-3,4-dihydroxy-5-(hydroxymethyl)tetrahydrofuran-2-yl)-7H-pyrrolo[2,3-d]pyrimidine-5-carbothioamide, 91

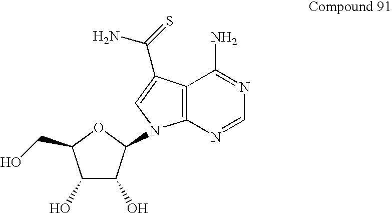

4-Amino-7-((2R,3R,4S,5R)-3,4-dihydroxy-5-(hydroxymethyl)tetrahydrofuran-2-yl)-7H-pyrrolo[2,3-d]pyrimidine-5-carbothioamide, 91 was prepared according to Townsend, et al., J. Het. Chem., 1988, Vol. 25, 1043-1046.

### Example 66

### Assay for Inhibition of HCV NS5B Polymerase

The following assay is used to measure the ability of the compounds of the present invention to inhibit the enzymatic activity of the RNA-dependent RNA polymerase (NS5B) of the hepatitis C virus (HCV) on a heteromeric RNA template.

### Procedure Assay Buffer Conditions: (50 μL-total/reaction)

### 20 mM Tris, pH 7.5

### 50 μM EDTA

### 5 mM DTT

### 2 mM MgCl₂

### 80 mM KCl

0.4 U/μL RNAsin (Promega, stock is 40 units/μL)
0.75 μg t500 (a 500-nt RNA made using T7 runoff transcription with a sequence from the NS2/3 region of the hepatitis C genome)
1.6 μg purified hepatitis C NS5B (form with 21 amino acids C-terminally truncated)
1 μM A, C, U, GTP (Nucleoside triphosphate mix)
[alpha-³²P]-GTP or [alpha-³³P]-GTP

The compounds are tested at various concentrations up to 100 μM final concentration. An appropriate volume of reaction buffer is made including enzyme and template t500. Nucleoside derivatives of the present invention are pipetted into the wells of a 96-well plate. A mixture of nucleoside triphosphates (NTP's), including the radiolabeled GTP, is made and pipetted into the wells of a 96-well plate. The reaction is initiated by addition of the enzyme-template reaction solution and allowed to proceed at room temperature for 1-2 h.

The reaction is quenched by addition of 20 μL 0.5M EDTA, pH 8.0. Blank reactions in which the quench solution is added to the NTPs prior to the addition of the reaction buffer are included.

L of the quenched reaction are spotted onto DE81 filter disks (Whatman) and allowed to dry for 30 mM. The filters are washed with 0.3 M ammonium formate, pH 8 (150 mL/wash until the cpm in 1 mL wash is less than 100, usually 6 washes). The filters are counted in 5-mL scintillation fluid in a scintillation counter.

The percentage of inhibition is calculated according to the following equation:

% Inhibition=[1−(cpm in test reaction−cpm in blank)/(cpm in control reaction−cpm in blank)]×100.

### Example 67

### Assay for Inhibition of HCV RNA Replication

The compounds of the present invention are evaluated for their ability to affect the replication of Hepatitis C Virus RNA in cultured hepatoma (HuH-7) cells containing a subgenomic HCV Replicon. This Replicon assay is a modification of that described in V. Lohmann, F. Korner, J-O. Koch, U. Herian, L. Theilmann, and R. Bartenschlager, “Replication of a Sub-genomic Hepatitis C Virus RNAs in a Hepatoma Cell Line,” Science 285:110 (1999).

### Protocol

The assay is an in situ Ribonuclease protection, Scintillation Proximity based-plate assay (SPA). 10,000-40,000 cells are plated in 100-200 μL of media containing 0.8 mg/mL G418 in 96-well cytostar plates (Amersham). Compounds are added to cells at various concentrations up to 100 μM in 1% DMSO at time 0 to 18 h and then cultured for 24-96 h. Cells are fixed (20 min, 10% formalin), permeabilized (20 min, 0.25% Triton X-100/PBS) and hybridized (overnight, 50° C.) with a single-stranded ³³P RNA probe complementary to the (+) strand NS5B (or other genes) contained in the RNA viral genome. Cells are washed, treated with RNAse, washed, heated to 65° C. and counted in a Top-Count. Inhibition of replication is read as a decrease in counts per minute (cpm).

Human HuH-7 hepatoma cells, which are selected to contain a subgenomic replicon, carry a cytoplasmic RNA consisting of an HCV 5′ non-translated region (NTR), a neomycin selectable marker, an EMCV IRES (internal ribosome entry site), and HCV non-structural proteins NS3 through NS5B, followed by the 3′ NTR.

### Example 68

### MT4 Cell Toxicity Assay

Cells used: MT4 cells were obtained from NIH AIDS research & Reference Reagent Program. Cells were used up to passage 30.

Toxicity assay using MTS: 5,000 MT4 cells were plated per well in a 96-well plate. Compounds were diluted in DMSO and further diluted with medium before adding to the plated cells. Plates were then incubated in a CO₂ incubator at 37° C. for 6 days.

Cell viability was estimated using MTS, a tetrazolium compound [3-(4,5-dimethylthiazol-2-yl)-5-(3-carboxymethoxyphenyl)-2-(4-sulfophenyl)-2H-tetrazolium], which is converted to a soluble formazan by mitochondrial dehydrogenases in the presence of an electron coupling reagent (phenazine methosulfate; PMS). The formazan product has an absorption maximum around 490 nm. Forty microliters of MTS reagent [MTS (2 mg/ml) and PMS (0.92 mg/ml) mixed at a ratio of 20:1 just before use] was added to each well and plates incubated for 4 hrs at 37° C. Absorbance was read at 490 nm using a Synergy 2 plate reader (BioTek). Data analysis was carried out using the BioTek Gen5 software using a 4-parameter curve fitting function to determine CC₅₀ values. See Table 2 below.

### Example 69

### Mitochondrial Biogenesis Assay

Cells used: HepG2 cells were obtained from ATCC. Cells are used up to 25 passages from the ATCC freeze.

Mitochondria Biogenesis Assay using HepG2 cells: 150,000 HepG2 cells were plated per well in a 6-well plate. Compounds were diluted in DMSO and further diluted in the plated cells so as to give a top concentration of 50 μM. Five four-fold dilutions were tested in this assay format. Plates were then incubated in a CO₂ incubator at 37° C. for 7 days. Medium containing fresh compound was changed on day 3 and 6 of the assay. Cells were harvested on day 7 of the assay.

On day 7, cells were rinsed with 1×DPBS three times and scraped off into 1×DPBS and held on ice. Whole cell protein estimation was determined to make sure that the cells are suspended at a protein concentration of 3-5 mg/ml.

Cells were lysed on ice by addition of 1:10 volume of diluted detergent (MitoSciences) so as to have a final detergent concentration of 2%. Cells were held on ice for 1 hr and then centrifuged at ˜20K×g for 20 min. The supernatant fraction was used for determining solubilized Frataxin and Complex IV using an ELISA kit from MitoSciences. The ratio of Complex IV to Frataxin was used to determine mitochondrial toxicity. DMSO controls were run to determine ratio of Frataxin and Complex IV in control cells. ddC was run as a positive control in the assay. See Table 2 below.

Table 2 shows anti-HCV activity and toxicity data for several compounds. The notation shown in Table 2 is described below.

### EC₅₀ Values

*=greater than or equal to 5 μM

### **=1-5 μM

### ***=0.1-1 μM

****=less than 0.1 μM

### CC₅₀ Values

### *=up to 1 μM

### **=1-20 μM

### ***=20-100 μM

****=greater than 100 μM

### MT-4 (TC₅₀) Values

### *=up to 1 μM

### **=1-20 μM

### ***=20-100 μM

****=greater than 100 μM

### Mitotoxicity

### *=up to 20 μM

### **=20-50 μM

### ***=50-80 μM

****=greater than 80 μM

TABLE 2

| | | | MT-4 (TC₅₀) | Mitotoxicity |
|---|---|---|---|---|
| | EC₅₀ (μM) | CC₅₀ (μM) | (μM) | (μM) |
| Compound a | **** | * | **** | * |
| Compound b | **** | * | * | * |
| Compound c | ***, * | *** | **, *** | — |
| Compound d | ** | — | ** | * |
| Compound e | ***, * | *** | **, *** | — |
| Compound f | * | — | **, *** | * |
| Compound g | **** | * | * | — |
| Compound h | **** | * | * | — |
| Compound j | *** | — | *** | ** |
| Compound k | *** | ** | *** | * |
| Compound l | *** | ** | **** | *** |
| Compound m | * | ***, **** | *** | *, ** |
| Compound n | ** | ** | **** | — |
| Compound o | ** | *** | — | — |
| Compound p | **** | * | * | * |
| Compound q | **** | ** | * | — |
| Compound r | ** | — | ** | * |

— not reported

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

What is claimed is:

1. A compound of Formula I 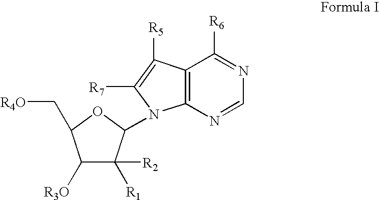
wherein:
R₁ and R₂ are independently selected from the group consisting of halogen, hydrogen, hydroxyl, N₃, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl and —NR′R″, wherein each occurrence of R′ and R″ are independently selected from the group consisting of hydrogen, hydroxyl, unsubstituted or substituted C₁₋₈ alkyl, unsubstituted or substituted C₁₋₈ alkoxyl, and unsubstituted or substituted C₃₋₆ cycloalkyl;
R₁ and R₂ are independently ORₓ, or OR_{y}; or
R₁ forms an unsubstituted or substituted 5-7 member ring with R₃ wherein said ring optionally comprises 1-2 additional heteroatoms selected from N, O or S; and
R₃, R₄, Rₓ and R_{y} are independently selected from the group consisting of
(a) hydrogen,
(b) unsubstituted or substituted C₁₋₈ alkyl, —C(═O)—Rₐ, —C(═O)—ORₐ or —C(═O)—NRₐRₐ′ wherein each occurrence of Ra and Ra′ are independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S;
(c) monophosphate, diphosphate or triphosphate,
(d) a moiety of Formula A, A′ or A″: 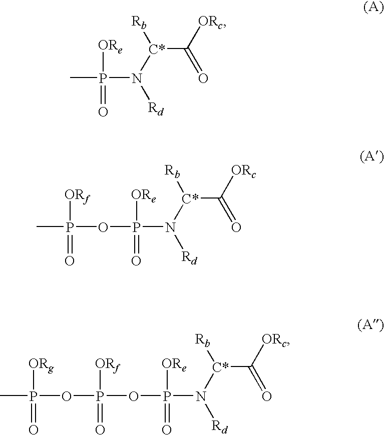
wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,
wherein R_{z} is hydrogen or unsubstituted or substituted C₁₋₈ alkyl;
R_{c}, R_{d}, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,
Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S,
R_{b} R_{d}, C* and N may form an unsubstituted or substituted 4-6 membered heterocycle comprising 1-3 additional heteroatoms selected from N, O or S;
(e) an amino acyl moiety of an amino acid;
(f) a moiety of Formula B, B′ or B″; 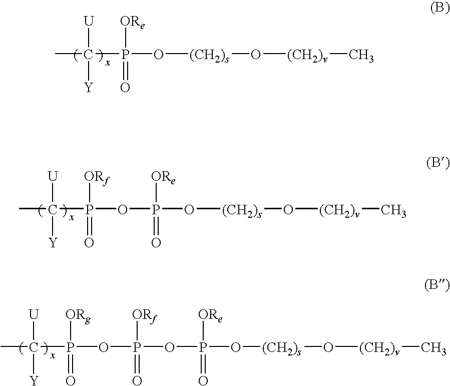
wherein U and Y are independently H or halogen, x is 0, 1 or 2, s is an integer from 2 to 6, v is an integer from 11 to 25, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S, and Rₑ is absent or independently selected from the group consisting of hydrogen, (CH₂)ₛ—O—(CH₂)ᵥ—CH₃, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S; or
R₃ and R₄ form a 5′,3′-cyclic phosphate as shown in Formula E: 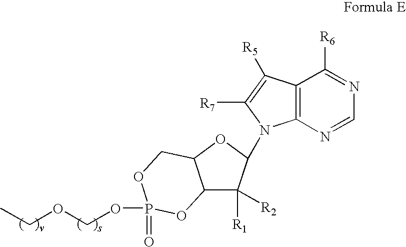
wherein s is an integer from 2 to 6 and v is an integer from 11 to 25; and
R₅, R₆ and R₇ are independently selected from the group consisting of hydrogen, halogen, hydroxyl, CN, unsubstituted or substituted C₁₋₈ alkyl, unsubstituted or substituted C₂₋₈ alkenyl, unsubstituted or substituted C₂₋₈ alkynyl, unsubstituted or substituted C₁₋₈ alkoxyl, unsubstituted or substituted C₁₋₈ thioalkyl, unsubstituted or substituted C₃₋₆ cycloalkyl, unsubstituted or substituted C₃₋₆ cycloalkenyl, unsubstituted or substituted C₆₋₁₄ aryl, unsubstituted or substituted heteroaryl comprising 1-4 heteroatoms selected from N, O and S, and —NRⁱRⁱⁱ, wherein at each occurrence Rⁱ and Rⁱⁱ are independently selected from the group consisting of hydrogen, hydroxyl, unsubstituted or substituted C₁₋₈ alkyl, unsubstituted or substituted C₁₋₈ alkenyl, unsubstituted or substituted C₁₋₈ alkynyl, unsubstituted or substituted C₁₋₈ alkoxyl and unsubstituted or substituted C₃₋₆ cycloalkyl, or
R₅, R₆ and R₇ are independently Formula C: 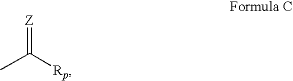
wherein Z is selected from the group consisting of O, S and NRⱼ, wherein Rⱼ is hydrogen, hydroxyl or unsubstituted or substituted C₁₋₈ alkoxyl; Rₚ is hydrogen, unsubstituted or substituted C₁₋₈ alkoxyl or —NRₘRₙ, wherein each occurrence of Rₘ or Rₙ are independently hydrogen, hydroxyl, unsubstituted or substituted C₁₋₈ alkyl, or unsubstituted or substituted C₁₋₈ alkoxyl;
or a pharmaceutically acceptable salt, prodrug, tautomer, regioisomer, stereoisomer, diastereomer, enantiomer or racemate thereof;
with the proviso that when R₂, R₃, R₄ and R₇ are each hydrogen, R₁ is hydroxyl and R₅ is C(═NOH)NH₂, then R₆ is not —NRⁱRⁱⁱ where Rⁱ is hydrogen and Rⁱⁱ is alkenyl substituted alkyl;
further with the proviso that when R₂ is N₃, R₁, R₃, R₄ and R₇ are each hydrogen, and R₆ is NH₂, then R₅ is not C(O)NH₂;
further with the proviso that when R₂ is methyl, R₁ is hydroxyl, R₃, R₄ and R₇ are each hydrogen, and R₅ is CN; then R₆ is not NH₂;
further with the proviso that when R₂, R₃, R₄ and R₇ are each hydrogen, R₁ is hydroxyl and R₅ is C(S)NH₂, then R₆ is not NH₂.

2. The compound of claim 1, wherein R₂ is hydrogen or unsubstituted or substituted C₁₋₈ alkyl and R₁ is hydrogen, hydroxyl or halogen.

3. The compound of claim 2, wherein R₄ is hydrogen.

4. The compound of claim 2, wherein R₃ and/or R₄ is unsubstituted or substituted C₁₋₈ alkyl, —C(═O)—Rₐ, —C(═O)—ORₐ or —C(═O)—NRₐRₐ′ wherein each occurrence of Rₐ and Rₐ′ are independently selected from the group consisting of unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

5. The compound of claim 2, wherein R₄ is a moiety of Formula A: 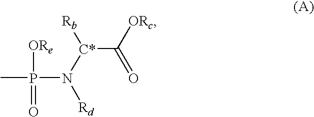
wherein R_{b} is selected from the group consisting of hydrogen, unsubstituted or substituted C₁₋₈ alkyl, unsubstituted or substituted C₁₋₈ thioalkyl, unsubstituted or substituted C₁₋₈ alkylthioalkyl, unsubstituted or substituted C₁₋₈ alkylthiol, unsubstituted or substituted amino-C₁₋₈-alkyl, unsubstituted or substituted aminocarbonyl-C₁₋₈-alkyl, —C(O)OR_{z}, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted heteroaryl-C₁₋₄-alkyl, unsubstituted or substituted C₆₋₁₄ aryl and unsubstituted or substituted heteroaryl comprising 1-4 heteroatoms selected from N, O and S,
wherein R_{z} is hydrogen or unsubstituted or substituted C₁₋₈ alkyl;
and R_{c}, R_{d} and Rₑ are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

6. The compound of claim 2, wherein R₄ is a moiety of Formula B, B′ or B″; 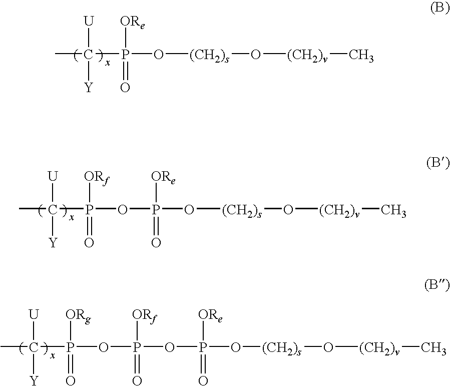
wherein U and Y are independently H or halogen, x is 0, 1 or 2, s is an integer from 2 to 6, v is an integer from 11 to 25, and Rₑ, R_{f} and R_{g} are absent or independently selected from the group consisting of hydrogen, unsubstituted or substituted C₁₋₈ alkyl, unsubstituted or substituted C₂₋₈ alkenyl, unsubstituted or substituted C₂₋₈ alkynyl, unsubstituted or substituted C₃₋₆ cycloalkyl, unsubstituted or substituted C₃₋₆ cycloalkenyl, unsubstituted or substituted C₆₋₁₄ aryl and unsubstituted or substituted heteroaryl comprising 1-4 heteroatoms selected from N, O and S.

7. The compound of claim 2, wherein R₃ and R₄ form a 5′,3′-cyclic phosphate as shown in Formula E: 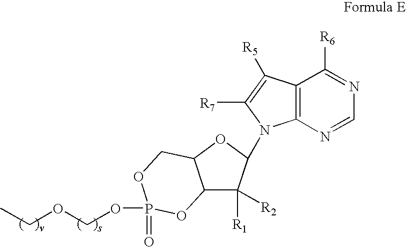
wherein s is an integer from 2 to 6 and v is an integer from 11 to 25.

8. The compound of claim 2, wherein said compound is selected from the group consisting of: 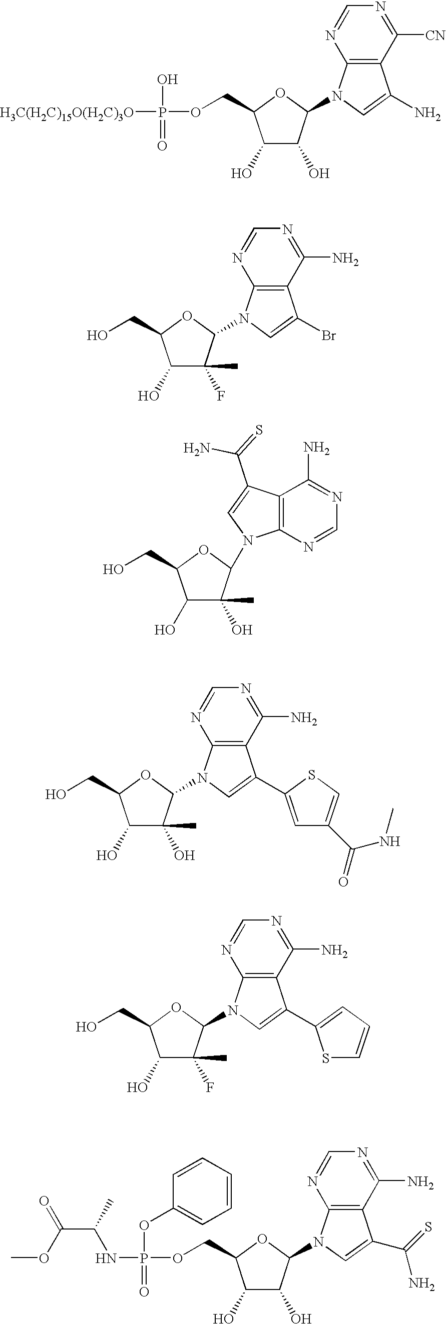 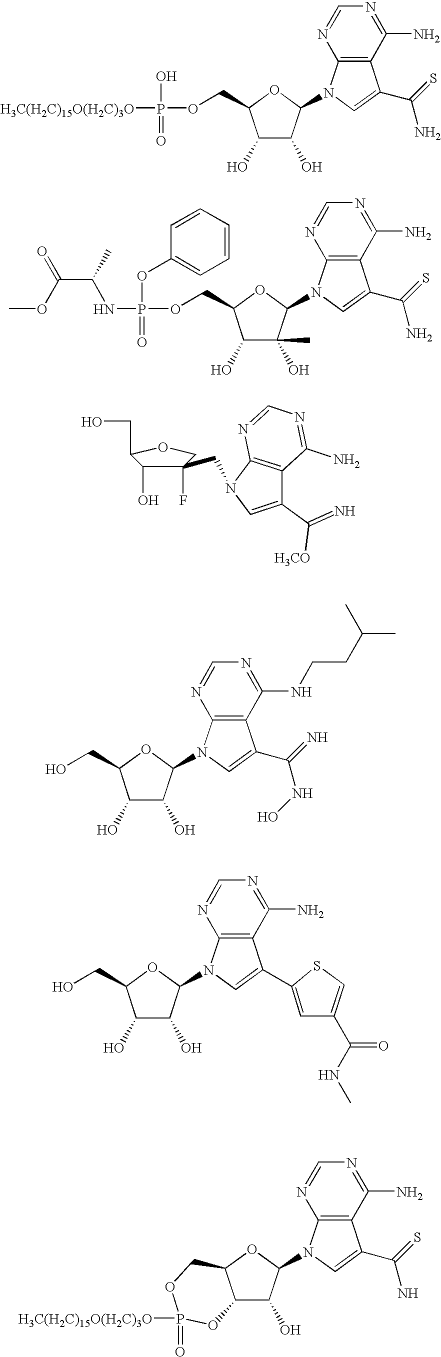 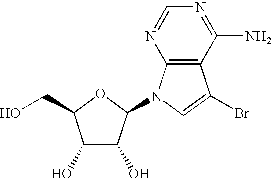
and pharmaceutically acceptable salts, prodrugs, tautomers, regioisomers, stereoisomers, diastereomers, enantiomers and racemates thereof.

9. The compound of claim 1, which exhibits an EC₅₀ of less than 5 μM against hepatitis C virus.

10. The compound of claim 9, which exhibits an EC₅₀ of less than 1 μM against hepatitis C virus.

11. The compound of claim 9, which further exhibits a TC₅₀ of greater than 1 μM.

12. A compound which exhibits an EC₅₀ of less than 5 μM against hepatitis C virus, wherein said compound is selected from the group consisting of: 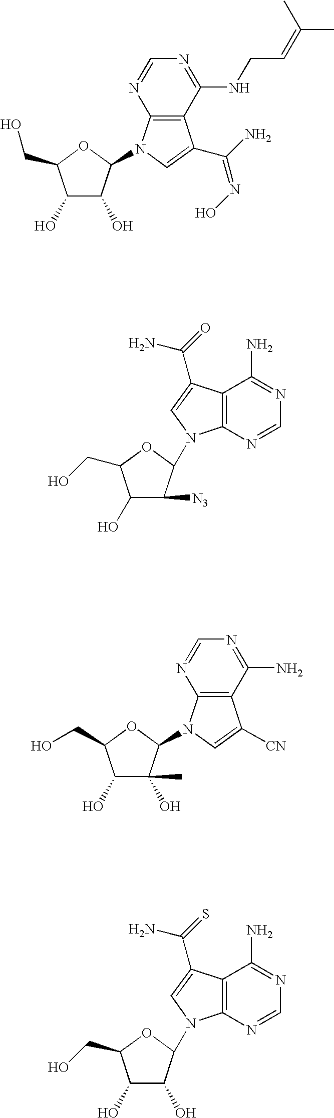
and pharmaceutically acceptable salts, prodrugs, tautomers, regioisomers, stereoisomers, diastereomers, enantiomers and racemates thereof.

13. The compound of claim 12, which exhibits an EC₅₀ of less than 1 μM against hepatitis C virus.

14. The compound of claim 12, which further exhibits a TC₅₀ of greater than 1 μM.

15. A pharmaceutical composition comprising a therapeutically effective amount of a compound of claim 1 and a pharmaceutically acceptable carrier.

16. A method for treating viral infection in a subject in need of such treatment, the method comprising administering to said subject a therapeutically effective amount of a compound of claim 1.

17. The method of claim 16, wherein said viral infection is caused by (+) Strand RNA virus or (−) Strand RNA virus.

18. The method of claim 16, wherein said viral infection is caused by Flaviviridae virus.

19. The method of claim 16, wherein said viral infection is caused by Hepatitis C virus (HCV).

20. The method of claim 16, wherein said compound is administered in combination with a therapeutically effective amount of one or more additional therapeutically active agents against Hepatitis C virus selected from the group consisting of nucleoside polymerase inhibitors, non-nucleoside polymerase inhibitors, protease inhibitors, NS4A inhibitors, immunomodulators, cyclophilin inhibitors, NS3 helicase inhibitors, α-glucosidase I inhibitors, Celgosivir (MX-3253), Debio 025, SCY-635, peginterferon alpha 2a, peginterferon alpha 2b, interferon 2a, ANA773, Nitazoxanide, GS-9190, VCH-759, VCH-222, HCV-796, ANA598, PF-00868554, IDX375, A-837093, GSK625433, BILN 1941, ACH 806, ACH-1095, MK-06080, R7128, R1626, Valopicitabine, IDX184, MK-7009, Boceprevir, Telaprevir, BI 201335, Telaprevir, ITMN-191, TMC435350, octadecyloxyethyl 9-(S)-[3-methoxy-2-(phosphonomethoxy)propyl]adenine, Pharmasset 7977, INX-08189, taribavirin and ribavirin.
